(19) United States
(12) Patent Application Publication
(10) Pub. No.: US 2008/0234280 A1
(43) Pub. Date: Sep. 25, 2008

(54) Use of Mc4 Receptor Agonist Compounds

(76) Inventors: Gordon McMurray, Kent (GB); Stephen Charles Phillips, Kent (GB); Simon Lempriere Westbrook, Kent (GB)

Correspondence Address: PFIZER INC., PATENT DEPARTMENT, MS8260-1611, EASTERN POINT ROAD, GROTON, CT, 06340, US

(21) Appl. No.: 11/996,543

(22) PCT Filed: Jul. 20, 2006
(86) PCT No.: PCT/IB06/02119

§ 371 Date: Jan. 23, 2008

Related U.S. Application Data
(60) Provisional application No. 60/705,237, filed on Aug. 2, 2005.

(30) Foreign Application Priority Data
Aug. 1, 2005 (GB) 05158175

(51) Int. Cl. A61K 31/501; A61K 31/454; A61P 13/02

(52) U.S. Cl. 514/252.03; 514/326

(57) ABSTRACT

This invention relates to the use of an MC4 receptor agonist compound for the manufacture of a medicament for the treatment of lower urinary tract dysfunction.

This application is a national filing of PCT/IB06/002119 filed Jul. 20, 2006 which claims priority to U.S. Provisional Application No. 60/705,237, filed Aug. 2, 2005 and UK Application Serial No. 0515817.5, filed Aug. 1, 2005.

The present invention relates to the use of melanocortin subtype-4 (MC4) receptor agonist compounds for the treatment of lower urinary tract dysfunction, including urinary incontinence (in particular stress urinary incontinence), overactive bladder (OAB), and lower urinary tract symptoms, particularly when associated with benign prostatic hyperplasia (LUTS associated with BPH).

The medical need is high for effective pharmacological treatments of lower urinary tract dysfunction. This high medical need is a result of lack of efficacious pharmacological therapy coupled with high patient numbers.

Urinary incontinence is the complaint of any involuntary leakage of urine. It is a common condition, and often constitutes an embarrassment which can lead to social isolation, depression, loss of quality of life, and is a major cause for institutionalisation in the elderly population. In addition, feelings of urge to urinate, nocturia, and an increased frequency of urination are conditions which also seriously compromise the quality of life of patients, and are also especially prevalent in the elderly population.

It is increasingly recognised that both supraspinal and spinal sites contain key neuroanatomical areas involved in the control of micturition. Pharmacological therapy may target the bladder directly, as is the case with muscarinic receptor antagonists used to treat OAB, alternatively the pharmacological therapy may target neuronal pathways controlling micturition, for example when SNRI's (serotonin-noradrenalin reuptake inhibitors) are used to treat SUI.

WO 2005/059558 (Bayer Healthcare AG, published 30 Jun. 2005) relates to methods for identifying therapeutic agents for diseases associated with MC4. Many disease areas are mentioned, including urinary disorders. However, the document does not disclose any compounds useful in such disorders and does not teach what interactions such compounds should have with the MC4 receptor.

WO 2005/077935 (International Patent Application No PCT/IB2005/000208, Pfizer, published 25 Aug. 2005, discloses a group of MC4 agonist compounds, but does not mention their use in the treatment of lower urinary tract dysfunction.

It has now been found that MC4 receptor agonists can be used for the treatment of lower urinary tract dysfunction.

Thus according to the broadest aspect of the present invention, there is provided the use of an MC4 receptor agonist compound for the manufacture of a medicament for the treatment of lower urinary tract dysfunction; and a method of treating lower urinary tract dysfunction which comprises administering an MC4 receptor agonist compound to a patient in need of such treatment.

The MC4 agonist compounds of WO 2005/077935 are suitable for use in the present invention. Thus, according to a preferred aspect of the present invention, the MC4 agonist compound is a compound of formula I,

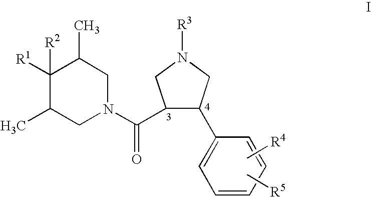

or a pharmaceutically acceptable salt, hydrate, solvate, isomer or prodrug thereof,

wherein R¹ is selected from: —(C₁-C₆)alkyl, —(C₂-C₆)alkenyl, —(C₂-C₆)alkynyl, —(C₃-C₈)cycloalkyl, —(C₅-C₈)cycloalkenyl, —(C₁-C₂)alkyl(C₃-C₈)cycloalkyl, aryl, —(C₁-C₂)alkylaryl, heterocyclic, or —(C₁-C₂)alkylheterocyclic groups
wherein each of the foregoing R¹ groups is optionally substituted by one or more groups selected from: —(C₁-C₄)alkyl, —(CH₂)ₘ(C₃-C₅)cycloalkyl, halogen, —(CH₂)ₘOR⁶, —CN, —C(O)OR⁶, —(CH₂)ₘNR⁷SO₂R⁸, CF₃, CH₂CF₃, OCF₃ or OCH₂CF₃ wherein m=0, 1 or 2;

R² is H, OH or OCH₃;

R³ is selected from: H, —(C₁-C₆)alkyl, —(C₂-C₆)alkenyl, —(C₂-C₆)alkynyl, —(C₃-C₈)cycloalkyl, —(C₅-C₈)cycloalkenyl, —(C₁-C₂)alkyl(C₃-C₈)cycloalkyl, aryl, —(C₁-C₂)alkylaryl, heterocyclic, or —(C₁-C₂)alkylheterocyclic groups
wherein each of the latter ten R³ groups is optionally substituted by one or more groups selected from: —OH, —(C₁-C₄)alkyl, —(CH₂)ₙ(C₃-C₅)cycloalkyl, halogen, —CN, —(CH₂)ₙOR⁶ or —(CH₂)ₙNR⁷R⁸ wherein n=0, 1 or 2;

R⁴ is selected from: —H, —(C₁-C₄)alkyl, —(C₂-C₄)alkenyl, —(C₂-C₄)alkynyl, —(CH₂)ₚ(C₃-C₅)cycloalkyl, —(CH₂)ₚ(C₅)cyclo-alkenyl, halogen, —(CH₂)ₚOR⁶, (CH₂)ₚNR⁷R⁸, —CN, —C(O)R⁶, —C(O)OR⁶, —C(O)NR⁷R⁸, —(CH₂)ₚNR⁷SO₂R⁸, CF₃, CH₂CF₃, OCF₃ or OCH₂CF₃ groups wherein p=0, 1 or 2;

R⁵ is selected from: —(C₁-C₄)alkyl, —(C₂-C₄)alkenyl, —(C₂-C₄)alkynyl, —(CH₂)ₚ(C₃-C₅)cycloalkyl, —(CH₂)ₚ(C₅)cyclo-alkenyl, halogen, —(CH₂)ₚOR⁶, —(CH₂)ₚNR⁷R⁸, —CN, —C(O)R⁶, —C(O)OR⁶, —C(O)NR⁷R⁸, —(CH₂)ₚNR⁷SO₂R⁸, CF₃, CH₂CF₃, OCF₃ or OCH₂CF₃ groups wherein p=0, 1 or 2;

or R⁴ and R⁵ can together form a fused 5- to 7-membered saturated or unsaturated ring;

R⁶, R⁷ and R⁸ are each independently selected from H, CH₃ or CH₂CH₃;

and wherein the heterocyclic groups of R¹ and R³ are independently selected from 4- to 10-membered ring systems containing up to 4 heteroatoms independently selected from O, N or S.

Heterocyclic groups suitable for use herein are 4- to 10-membered mono or bicyclic heteroaryl rings containing one to three heteroatoms from the list N, S and O and combinations thereof and wherein said bicyclic heteroaryl rings are 9- or 10-membered ring systems which may be either two heteroaryl rings fused together or a heteroaryl ring fused to an aryl ring.

Suitable bicyclic heteroaryl groups for use herein include: include: benzimidazolyl, benzotriazolyl, benzothiazolyl, indazolyl, indolyl, imidazopyridinyl, imidazopyrimidinyl, pyrrolopyridinyl, quinolinyl, isoquinolinyl, quinazolinyl, naphthyridinyl and pyridopyrimidinyl groups.

Preferred for use herein are monocyclic 5- to 6-membered heteroaryl rings containing one or three heteroatoms from the list N and O and combinations thereof.

Suitable 5-membered ring monocyclic heteroaryl groups for use herein include: triazinyl, oxadiazinyl, oxazolyl, thiazolyl, thiadiazolyl, furyl, thienyl and pyrrolyl and imidazolyl groups.

Suitable 6-membered ring monocyclic heteroaryl groups for use herein include: pyridinyl, pyrimidinyl, pyridazinyl and pyrazinyl groups.

Preferred R¹ heterocyclic rings are monocyclic 5- to 6-membered heteroaryl rings containing one or two heteroatoms from the list N and O and combinations thereof. More preferred R¹ heterocyclic rings are monocyclic 5- to 6-membered heteroaryl rings containing one or 2 N heteroatoms. Highly preferred R¹ heterocyclic rings herein are monocyclic 6-membered heteroaryl rings containing one or two N heteroatoms such as pyridinyl and pyrimidinyl.

An especially preferred R¹ heteroaryl group herein is the pyridinyl group.

Preferred R³ heterocyclic rings are monocyclic 5- to 6-membered heteroaryl rings containing one or two heteroatoms from the list N and O and combinations thereof such as tetrahydropyranyl, pyridinyl, pyridazinyl, pyrazinyl and pyrimidinyl groups. More preferred R³ heterocyclic rings are monocyclic 5- to 6-membered heteroaryl rings containing one or two N heteroatoms. More preferred still as R³ heterocyclic rings are monocyclic 6-membered heteroaryl rings containing one or two N heteroatoms such as pyridinyl, pyridazinyl, pyrazinyl and pyrimidinyl groups.

Particularly preferred R³ 6-membered ring monocyclic heteroaryl groups for use herein are pyridin-2-yl, pyridin-3-yl, pyridazin-3-yl, pyrazinyl, pyrimidin-5-yl and pyrimidin-2-yl groups. Especially preferred R³ 6-membered ring monocyclic heteroaryl groups for use herein include pyridin-2-yl, pyridin-3-yl and pyridazin-3-yl groups. Of these groups pyridazin-3-yl is most preferred.

Suitable fused ring systems formed by R⁴ and R⁵ together may be carbocyclic ring systems or heterocyclic ring systems containing up to two heteroatoms selected from O, N or S. Including the phenyl ring to which they are attached, preferred ring systems which R⁴ and R⁵ may form are: indane, 1,2,3,4-tetrahydronaphthalene, indolyl, indazolyl, naphthyl, quinolyl, benzothiazolyl, benzimidazolyl, benzo[1,3]dioxolane, 2,3-dihydrobenzo[1,4]dioxine, 2,3-dihydrobenzofuran, 2,3-dihydrobenzothiophene and 1,3-dihydroisobenzofuran.

In the above definitions, unless otherwise indicated, alkyl, alkenyl and alkynyl groups having three or more carbon atoms, and alkanoyl groups having four or more carbon atoms, may be straight chain or branched chain. For example, a C₄ alkyl substituent can be in the form of normal-butyl (n-butyl), iso-butyl (i-butyl), secondary-butyl (sec-butyl) or tertiary-butyl (t-butyl). For the avoidance of doubt where R¹ and/or R³ is an optionally substituted alkyl group said alkyl group(s) may not be further substituted by a further (unsubstituted) alkyl group. Furthermore where R³ is substituted with an alkenyl or an alkynyl group the carbon atom (of said unsaturated group), which is directly bonded to the N atom, may not itself be unsaturated.

The term halogen includes Cl, Br, F, and I.

The term “aryl”, when used herein, includes six- to ten-membered carbocyclic aromatic groups, such as phenyl and naphthyl.

The pharmaceutically acceptable salts of the compounds of the formula (I) include the acid addition and the base salts thereof. The preparation of the salt forms and examples thereof are given in PCT/IB2005/000208 (published as WO 2005/077935 mentioned above). The compounds used in the invention include compounds of formula (I) as hereinbefore defined, polymorphs and crystal habits thereof, prodrugs, and isomers thereof (including optical, geometric and tautomeric isomers) as hereinafter defined and isotopically labelled compounds of formula (I).

Specifically included within the scope of the present invention is the use of stereoisomeric mixtures of compounds having formula (I), or a diastereomerically enriched or diastereomerically pure isomer of a compound of formula (I), or an enantiomerically enriched or enantiomerically pure isomer of a compound of formula (I).

Preferred groups of compounds of formula I include those in which:
(a) R¹ is selected from: —(C₁-C₆)alkyl, —(C₃-C₈)cycloalkyl, —(C₁-C₂)alkyl(C₃-C₈)cycloalkyl, phenyl, —(C₁-C₂)alkylaryl, heterocyclic, or —(C₁-C₂)alkylheterocyclic groups
   wherein each of the foregoing R¹ groups is optionally substituted by one or more groups selected from: —(C₁-C₄)alkyl, halogen, —(CH₂)ₘOR⁶, CN, CF₃ or OCF₃, wherein m=1 or 2;
   R² is OH;
   R³ is selected from: —H, —(C₁-C₆)alkyl, —(C₃-C₈)cycloalkyl, —(C₁-C₂)alkyl(C₃-C₈)cycloalkyl, aryl, —(C₁-C₂)alkylaryl, heterocyclic, or —(C₁-C₂)alkylheterocyclic groups
   wherein each of the latter seven R³ groups is optionally substituted by one or more groups selected from: —OH, —(C₁-C₄)alkyl, —(CH₂)ₙ(C₃-C₅)cycloalkyl, halogen, CN, —(CH₂)ₙOR⁶ or —(CH₂)ₙNR⁷R⁸ wherein n=0, 1 or 2;
   R⁴ is selected from: —H, —(C₁-C₄)alkyl, —(CH₂)ₚ(C₃-C₅)cycloalkyl, halogen, —(CH₂)ₚOR⁶, —(CH₂)ₚNR⁷R₈, —CN, —C(O)R⁶, —C(O)OR⁶, —C(O)NR⁷R⁸, —(CH₂)ₚNR⁷SO₂R⁸, CF₃, CH₂CF₃, OCF₃ or OCH₂CF₃ groups wherein p=0, 1 or 2;
   R⁵ is selected from: —(C₁-C₄)alkyl, —(CH₂)ₚ(C₃-C₅)cycloalkyl, halogen, —(CH₂)ₚOR⁶, (CH₂)ₚNR⁷R⁸, CN, C(O)R⁶, C(O)OR⁶, CONR⁷R⁸, (CH₂)ₚNR⁷SO₂R⁸, CF₃, CH₂CF₃, OCF₃ or OCH₂CF₃ groups wherein p=0, 1 or 2;
   R⁶, R⁷ and R⁸ are each independently selected from H, CH₃ or CH₂CH₃;
   wherein the heterocyclic group of R³ is selected from mono-cyclic 5- to 6-membered ring systems containing up to 2 heteroatoms independently selected from O or N and combinations thereof,
   and wherein the heterocyclic group of R¹ is selected from mono-cyclic 5- to 6-membered ring systems containing up to 1 heteroatoms independently selected from O or N;
(b) R¹ is selected from n-propyl, i-propyl, n-butyl, methoxymethyl, cyclopropyl, cyclohexyl, phenyl, 3-fluorophenyl, 4-fluorophenyl, 4-chlorophenyl, 4-methylphenyl, 4-methoxyphenyl, 2,6-difluorophenyl, 2,4-difluorophenyl, 3,4-difluorophenyl, pyridin-2-yl or pyridin-3-yl groups;
(c) R³ is —H, —(C₂-C₆)alkyl, —(C₃-C₈)cycloalkyl, —(C₁-C₂)alkyl(C₃-C₈)cycloalkyl or heterocyclic wherein each of the latter four R³ groups is optionally substituted by one or more groups selected from —OH, —(C₁-C₄)alkyl or —OR⁶ wherein R⁶ is —H, CH₃ or CH₂CH₃ and wherein when R³ is a heterocyclic group said heterocyclic group is a monocyclic 6-membered ring system containing up to 2 N heteroatoms;
(d) R³ is selected from: hydrogen, ethyl, i-propyl, n-propyl, n-butyl, t-butyl, i-butyl, 2-methoxyethyl, cyclopentyl, cyclobutyl, cyclopentylmethyl, pyridin-2-yl, pyridin-3-yl, pyridazin-3-yl, pyrazinyl, pyrimidin-5-yl, pyrimidin-2-yl, pyrimidin-4-yl or tetrahydropyran-4-yl groups;
(e) R⁴ is selected from H, F or Cl and R⁵ is selected from F or Cl; and
(f) the compound is of general formula (IC),

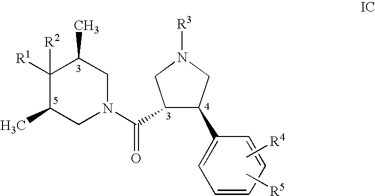

wherein:
R¹ is a phenyl, 3-fluorophenyl, 4-fluorophenyl, 2,6-difluorophenyl, 2,4-difluorophenyl, 3,4-difluorophenyl or pyridin-2-yl group;
R² is OH;
R³ is t-butyl;
R⁴ is selected from: H or F and R⁵ is selected from: F or Cl.

Preferred compounds for use in the present invention include:

(3R,4R,5S)-1-{[(3S,4R)-1-tert-Butyl-4-(2,4-difluorophenyl)pyrrolidin-3-yl]carbonyl}-3,5-dimethyl-4-phenylpiperidin-4-ol;

(3R,4R,5S)-1-{[(3S,4R)-1-tert-Butyl-4-(2,4-difluorophenyl)pyrrolidin-3-yl]carbonyl}-3,5-dimethyl-4-phenylpiperidin-4-ol hydrochloride;

(3R,4R,5S)-1-{[(3S,4R)-4-(2,4-Difluorophenyl)-1-isopropylpyrrolidin-3-yl]carbonyl}-3,5-dimethyl-4-phenylpiperidin-4-ol hydrochloride;

(3R,4R,5S)-1-{[(3S,4R)-1-tert-Butyl-4-(2,4-difluorophenyl)pyrrolidin-3-yl]carbonyl}-4-(3,4-difluorophenyl)-3,5-dimethylpiperidin-4-ol hydrochloride;

(3R,4R,5S)-1-{[(3S,4R)-1-tert-butyl-4-(2,4-difluorophenyl)pyrrolidin-3-yl]carbonyl}-4-(4-fluorophenyl)-3,5-dimethylpiperidin-4-ol hydrochloride;

(3R,4R,5S)-1-{[(3S,4R)-4-(2,4-difluorophenyl)-1-isopropylpyrrolidin-3-yl]carbonyl}-4-(4-fluorophenyl)-3,5-dimethylpiperidin-4-ol hydrochloride;

(3R,4R,5S)-1-{[(3S,4R)-4-(2,4-difluorophenyl)pyrrolidin-3-yl]carbonyl}-4-(4-fluorophenyl)-3,5-dimethylpiperidin-4-ol hydrochloride;

(3R,4R,5S)-1-{[(3S,4R)-1-tert-butyl-4-(2,4-difluorophenyl)pyrrolidin-3-yl]carbonyl}-4-(4-chlorophenyl)-3,5-dimethylpiperidin-4-ol hydrochloride;

(3R,4R,5S)-1-{[(3S,4R)-1-tert-butyl-4-(2,4-difluorophenyl)pyrrolidin-3-yl]carbonyl}-4-(2,4-difluorophenyl)-3,5-dimethylpiperidin-4-ol hydrochloride;

(3R,4R,5S)-4-(2,4-difluorophenyl )-1-{[(3S,4R)-4-(2,4-difluorophenyl)pyrrolidin-3-yl]carbonyl}-3,5-dimethylpiperidin-4-ol hydrochloride;

(3R,4R,5S)-1-{[(3S,4R)-1-tert-Butyl-4-(2,4-difluorophenyl)pyrrolidin-3-yl]carbonyl}-3,5-dimethyl-4-pyridin-2-ylpiperidin-4-ol hydrochloride;

(3R,4R,5S)-1-{[(3S,4R)-4-(2,4-Difluorophenyl)pyrrolidin-3-yl]carbonyl}-3,5-dimethyl-4-phenylpiperidin-4-ol hydrochloride;

(3R,4R,5S)-1-{[(3S,4R)-4-(2,4-Difluorophenyl)-1-pyridin-2-ylpyrrolidin-3-yl]carbonyl}-3,5-dimethyl-4-phenylpiperidin-4-ol hydrochloride;

(3R,4R,5S)-1-{[(3S,4R)-4-(2,4-Difluorophenyl)-1-pyridin-3-ylpyrrolidin-3-yl]carbonyl}-3,5-dimethyl-4-phenylpiperidin-4-ol hydrochloride;

(3R,4R,5S)-1-{[(3S,4R)-4-(2,4-Difluorophenyl)-1-pyridazin-3-ylpyrrolidin-3-yl]carbonyl}-3,5-dimethyl-4-phenylpiperidin-4-ol hydrochloride;

(3R,4R,5S)-1-{[(3S,4R)-1-tert-Butyl-4-(2,4-difluorophenyl)pyrrolidin-3-yl]carbonyl}-3,5-dimethyl-4-propylpiperidin-4-ol hydrochloride;

(3R,4R,5S)-1-{[(3S,4R)-4-(2,4-Difluorophenyl)-1-pyrimidin-4-ylpyrrolidin-3-yl]carbonyl}-3,5-dimethyl-4-phenylpiperidin-4-ol hydrochloride;

(3R,4R,5S)-1-{[(3S,4R)-4-(2,4-Difluorophenyl)-1-pyridazin-3-ylpyrrolidin-3-yl]carbonyl}-3,5-dimethyl-4-pyridin-2-ylpiperidin-4-ol hydrochloride;

(3R,4R,5S)-1-{[(3S,4R)-4-(4-Chlorophenyl)pyrrolidin-3-yl]carbonyl}-3,5-dimethyl-4-phenylpiperidin-4-ol hydrochloride;

(3R,4R,5S)-4-(4-chlorophenyl)-1-{[(3S,4R)-4-(2,4-difluorophenyl)-1-isopropylpyrrolidin-3-yl]carbonyl}-3,5-dimethylpiperidin-4-ol hydrochloride;

(3R,4R,5S)-4-(3,4-difluorophenyl)-1-{[(3S,4R)-4-(2,4-difluorophenyl)-1-isopropylpyrrolidin-3-yl]carbonyl}-3,5-dimethylpiperidin-4-ol hydrochloride;

(3R,4R,5S)-4-(2,4-difluorophenyl)-1-{[(3S,4R)-4-(2,4-difluorophenyl)-1-isopropylpyrrolidin-3-yl]carbonyl}-3,5-dimethylpiperidin-4-ol hydrochloride;

(3R,4R,5S)-1-{[(3S,4R)-4-(2,4-difluorophenyl)-1-ethylpyrrolidin-3-yl]carbonyl}-4-(3-fluorophenyl)-3,5-dimethylpiperidin-4-ol hydrochloride

and pharmaceutically acceptable acid salts, solvates and hydrates thereof.

Preferred compounds for use in the present invention are independently selected from the group consisting of:

(3R,4R,5S)-1-{[(3S,4R)-1-tert-Butyl-4-(2,4-difluorophenyl)pyrrolidin-3-yl]carbonyl}-3,5-dimethyl-4-phenylpiperidin-4-ol;

(3R,4R,5S)-1-{[(3S,4R)-1-tert-Butyl-4-(2,4-difluorophenyl)pyrrolidin-3-yl]carbonyl}-3,5-dimethyl-4-phenylpiperidin-4-ol hydrochloride;

(3R,4R,5S)-1-{[(3S,4R)-1-tert-Butyl-4-(2,4-difluorophenyl)pyrrolidin-3-yl]carbonyl}-4-(3,4-difluorophenyl)-3,5-dimethylpiperidin-4-ol hydrochloride;

(3R,4R,5S)-1-{[(3S,4R)-1-tert-butyl-4-(2,4-difluorophenyl)pyrrolidin-3-yl]carbonyl}-4-(4-fluorophenyl)-3,5-dimethylpiperidin-4-ol hydrochloride;

(3R,4R,5S)-1-{[(3S,4R)-4-(2,4-difluorophenyl)pyrrolidin-3-yl]carbonyl}-4-(4-fluorophenyl)-3,5-dimethylpiperidin-4-ol hydrochloride;

(3R,4R,5S)-1-{[(3S,4R)-1-tert-butyl-4-(2,4-difluorophenyl)pyrrolidin-3-yl]carbonyl}-4-(4-chlorophenyl)-3,5-dimethylpiperidin-4-ol hydrochloride;

(3R,4R,5S)-4-(4-chlorophenyl)-1-{[(3S,4R)-4-(2,4-difluorophenyl)-1-isopropylpyrrolidin-3-yl]carbonyl}-3,5-dimethylpiperidin-4-ol hydrochloride

and pharmaceutically acceptable acid salts, solvates and hydrates thereof.

More preferably, the compound of formula I is (3R,4R,5S)-1-{[(3S,4R)-1-tert-Butyl-4-(2,4-difluorophenyl)pyrrolidin-3-yl]carbonyl}-3,5-dimethyl-4-phenylpiperidin-4-ol also known as [1-tert-Butyl-4-(2,4-difluoro-phenyl)-pyrrolidin-3-yl]-(4-hydroxy-3,5-dimethyl-4-phenyl-piperidin-1-yl)-methanone (the compound of Example 1 of WO 2005/077935), having the formula,

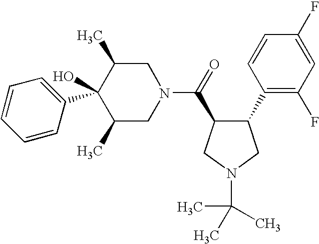

or a pharmaceutically acceptable salt, hydrate, solvate, isomer or prodrug thereof.

The preparation of the compounds of formula I, as well as teachings as to their formulation, dosage and routes of administration, are described in PCT/IB2005/000208 (now published as WO 2005/077935, mentioned above), which is incorporated herein by reference in its entirety.

Prodrugs include pharmaceutically acceptable esters and amides formed by any carboxylic acid, hydroxy and amine groups present in the molecule with C₁₋₆ alcohols or carboxylic acids which hydrolyze in vivo to give the original carboxylic acid, hydroxy and amine groups.

Co-pending Provisional U.S. Patent Application 60/706,191 (filed 4 Aug. 2005, inventor Mark David Andrews et al, applicant's reference PC 33020, incorporated herein by reference) discloses a further group of melanocortin type 4 receptor agonist compounds suitable for use in the present invention. Thus according to a second preferred aspect of the invention, the MC4 agonist compound has the general formula (Ia),

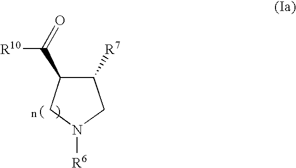

wherein:

n is 1 or 2;

R⁶ is selected from H, C₁-C₆alkyl, C₃-C₈cycloalkyl, aryl, heterocyclyl, heteroaryl, C(O)C₁-C₆alkyl and CO₂C₁-C₆alkyl, wherein said moieties may be optionally substituted with one or more substituents independently selected from halo, CN, C₁-C₄alkyl and C₁-C₄alkoxy;

R⁷ is selected from pyridinyl and phenyl, wherein said pyridinyl or said phenyl is substituted by 1-3 groups independently selected from halo, CN, CF₃, OCF₃, OC₁-C₄alkyl and C₁-C₄alkyl;

R¹⁰ is a substituted piperidine group of formula (IIa):

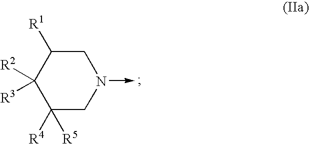

wherein
R¹ and R⁴ are each independently selected from H, C₁-C₄alkyl, OH, O(C₁-C₄alkyl), CH₂OCH₃ and NR⁸R⁹;
R² is selected from H, OH, OC₁-C₄alkyl and NR⁸R⁹;
R³ is selected from aryl or heteroaryl, wherein said moieties are optionally substituted with one or more substituents independently selected from halo, CN, CF₃, OCF₃, O(C₁-C₄alkyl), and C₁-C₄alkyl;
R⁵ is selected from H and C₁-C₄alkyl;
R⁸ is selected from H and C₁-C₄alkyl, wherein said C₁-C₄alkyl is optionally substituted with OH or OCH₃;
R⁹ is selected from H, C₁-C₄alkyl, SO₂C₁-C₄alkyl, C(O)C₁-C₄alkyl;

wherein aryl means a six or ten membered aromatic hydrocarbon ring which is optionally fused to another six or ten membered aromatic hydrocarbon ring;

wherein heteroaryl means a 5 or 6 membered aromatic ring, containing from 1 to 4 heteroatoms, said heteroatoms each independently selected from O, S and N, wherein said aromatic ring may be optionally fused to an aryl or second, non-fused, aromatic heterocyclic ring;

wherein heterocyclyl means a 4 to 7 membered saturated or partially saturated ring, containing from 1 to 2 heteroatoms each independently selected from O, S and N;

wherein halo means Cl, F, Br or I;

and pharmaceutically acceptable salts, hydrate, solvates, polymorphs and prodrugs thereof, with the provisos that:

R¹, R⁴ and R⁵ are not all simultaneously be H;

when R¹ is methyl and R⁴ is H, then R⁵ is not methyl;

when R⁴ is methyl and R⁵ is H, then R¹ is not methyl; and

when R⁵ is methyl and R⁴ is H, then R¹ is not methyl.

The preparation of compounds of formula la is described below and in the Examples.

Alkyl is straight chain or branched.

Suitable aryl groups include phenyl and naphthyl.

Suitable heteroaryl groups include pyridinyl, pyrimidinyl, pyridazinyl and pyrazinyl, pyrrolyl, furanyl, pyrazolyl, imidazolyl, oxazolyl, isoxazolyl, thiazolyl, tetrazolyl, 1,2,3-triazolyl, 1,3,4-triazolyl, indolyl, indazolyl, pyrrolopyridinyl, pyrrolopyrimidinyl, benzimidazolyl, isoquinolinyl and quinolinyl.

Suitable heterocyclyl groups include azetidinyl, tetrahydrofuranyl, pyrrolidinyl, tetrahydropyranyl, piperidinyl, piperazinyl, dihydropyranyl and tetrahydropyridinyl.

Unless otherwise indicated, the term “substituted” means substituted by one or more defined groups. In the case where groups may be selected from a number of alternatives groups, the selected groups may be the same or different.

Compounds of formula (Ia) contain two or more asymmetric carbon atoms and therefore exist in different stereoisomeric forms. Furthermore, the skilled person will understand that the present invention encompasses all stereoisomeric and diastereoisomeric forms, in particular compounds of general formula (IaA), (IaB), (IaC), (IaD), (IaE), (IaF), (IaG) and (IaH):

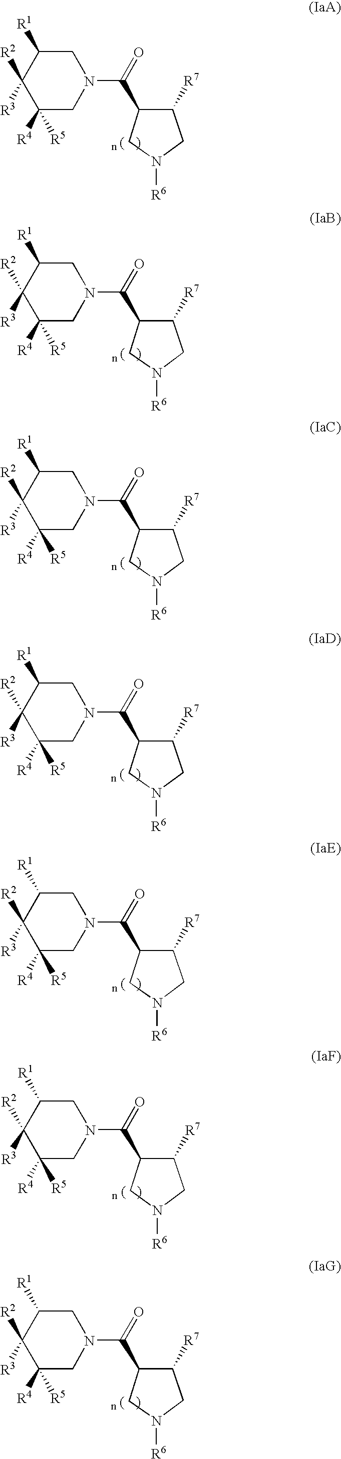

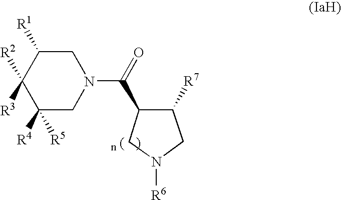

Separation of diastereoisomers may be achieved by conventional techniques, e.g. by fractional crystallisation, chromatography or H.P.L.C. of a stereoisomeric mixture of a compound of formula (IaA), (IaB), (IaC), (IaD), (IaE), (IaF), (IaG) or (IaH) or a suitable salt or derivative thereof. An individual enantiomer of a compound of formula (IaA), (IaB), (IaC), (IaD), (IaE), (IaF), (IaG) or (IaH) may also be prepared from a corresponding optically pure intermediate or by resolution, such as by H.P.L.C. of the corresponding racemate using a suitable chiral support or by fractional crystallisation of the diastereoisomeric salts formed by reaction of the corresponding racemate with a suitable optically active acid or base, as appropriate.

In a preferred group of compounds of formula Ia:

n is 1;

R¹ is selected from H, methyl, OH, OCH₃ and OC₂H₅;

R² is selected from OH, OCH₃ and OC₂H₅;

R³ is selected from phenyl or pyridinyl, wherein said moieties are optionally substituted with one or more substituents independently selected from F, Cl, CN and CF₃;

R⁴ is selected from H, methyl, OH, OCH₃ and OC₂H₅;

R⁵ is selected from H and methyl;

R⁶ is selected from C₁-C₄alkyl, tetrahydropyranyl, tetrahydrofuranyl, pyrimidinyl pyridinyl and pyridazinyl, wherein each of said moieties is optionally substituted with one or more substituents independently selected from halo, CN, methyl and OCH₃;

R⁷ is selected from pyridinyl and phenyl, wherein said pyridinyl or said phenyl is substituted by 1-2 groups independently selected from Cl, F, CN and OCH₃;

R⁸ is selected from H, methyl and ethyl; and

R⁹ is selected from H and methyl.

Preferably, R⁶ is selected from the following group:

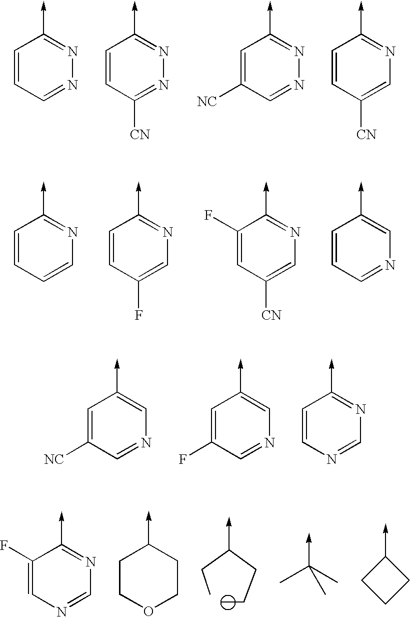

Preferably, R⁷ is selected from the following group:

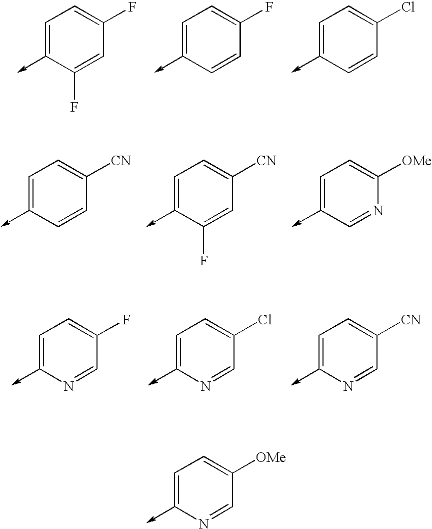

Preferably, R¹⁰ is selected from the following group:

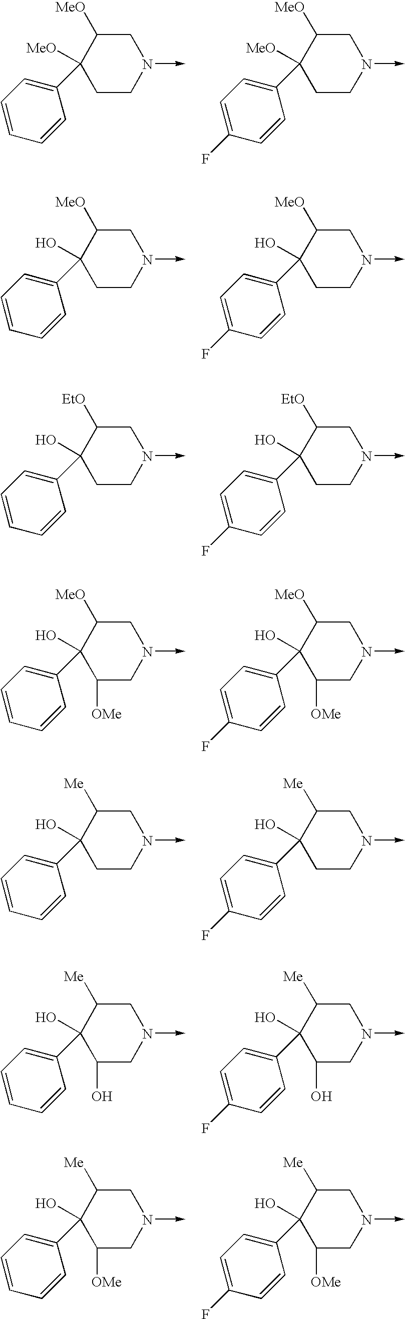

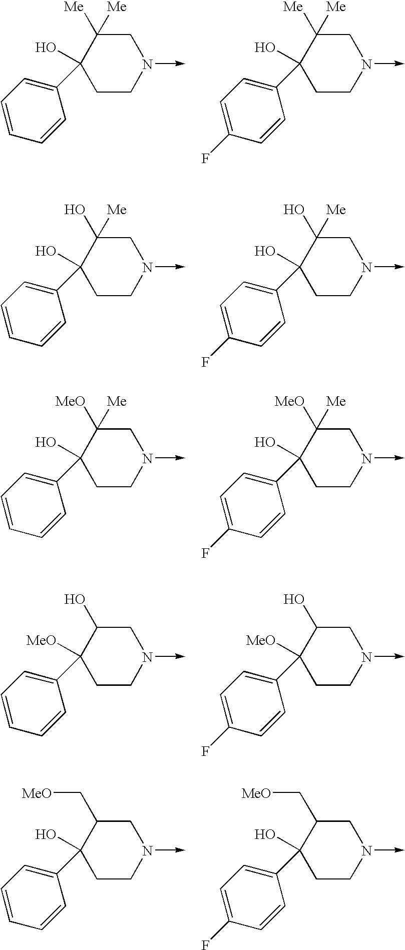

A preferred compound of formula Ia is

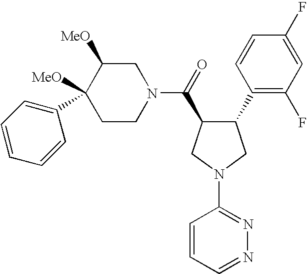

or a pharmaceutically acceptable salt, hydrate, solvate, polymorph or prodrug thereof (see Example 8 below).

WO 2006/019787 (Merck & Co, Inc) discloses a group of MC4 receptor agonists. However, their use in the treatment of lower urinary tract dysfunction is not mentioned. Thus, according to a further preferred aspect of the invention, the MC4 receptor agonist is a compound of formula (Ib),

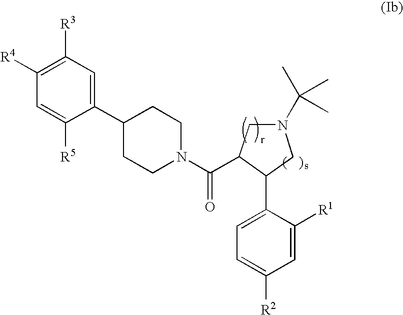

or a pharmaceutically acceptable salt thereof; wherein

R¹ and R² are selected from the group consisting of:
(1) halogen,
(2) CF₃,
(3) CH₃, and
(4) OCH₃;

R³ and R⁴ are independently selected from the group consisting of:
(1) C₁₋₄ alkyl,
(2) —CF₃,
(3) halogen,
(4) —OC₁₋₄ alkyl,
(5) —OCF₃,
(6) —OCHF₂,
(7) —S(O)pC₁₋₄ alkyl, and
(8) —CN,

wherein alkyl is unsubstituted or substituted with one to three substituents independently selected from halogen, hydroxy, oxo, C₁₋₄ alkyl, trifluoromethyl, and C₁₋₄ alkoxy, or wherein the R³ and R⁴ substitutents taken together with the carbons to which they are attached form a 4-6 membered ring optionally containing a heteroatom selected from O, S, —NH, and —NC₁₋₄alkyl;

R⁵is selected from the group consisting of:
(1) —C₁₋₈ alkyl,
(2) —(CH₂)ₙ-heteroaryl,
(3) —(CH₂)ₙheterocycloalkyl,
(4) halogen,
(5) —OR⁶,
(6) —(CH₂)ₙC(O)R⁶,
(7) —(CH₂)ₙOC(O)R⁶,
(8) —(CH₂)ₙC(O)OR⁶,
(9) —(CH₂)ₙC≡N,
(10) —(CH₂)ₙN(R⁶)₂,
(11) —(CH₂)ₙC(O)N(R⁶)₂,
(12) —(CH₂)ₙNR⁶C(O)R⁶,
(13) —(CH₂)ₙNR⁶C(O)OR⁶,
(14) —(CH₂)ₙNR⁶C(O)-heteroaryl,
(15) —(CH₂)ₙNR⁶C(O)N(R⁶)₂,
(16) —(CH₂)ₙNR⁶-heteroaryl,
(17) —(CH₂)ₙC(O)NR⁶N(R⁶)₂,
(18) —(CH₂)ₙC(O)NR⁶NR⁶C(O)R⁶,
(19) —(CH₂)ₙNR⁶S(O)ₚR⁶,
(20) —(CH₂)ₙS(O)ₚN(R⁶)₂,
(21) —(CH₂)ₙS(O)ₚR⁶,
(22) —O(CH₂)ₙC(O)N(R⁶)₂,
(23) —(CH₂)ₙCF₃, and
(24) —O(CH₂)ₙCF₃,

wherein heteroaryl is unsubstituted or substituted with one to three substituents independently selected from halogen, hydroxy, C₁₋₄ alkyl, trifluoromethyl, and C₁₋₄ alkoxy, and wherein any alkyl, heterocycloalkyl, and methylene (CH₂) carbon atom in R⁵ is unsubstituted or substituted with one to two substituents independently selected from halogen, hydroxy, oxo, C₁₋₄ alkyl, trifluoromethyl, and C₁₋₄ alkoxy, or two substituents on the same R⁵ carbon atom are taken together with the carbon atom to form a 3- to 6-membered ring;

each R⁶ is independently selected from the group consisting of:
(1) hydrogen,
(2) C₁₋₈ alkyl,
(3) phenyl,
(4) heteroaryl,
(5) —(CH₂)ₙheterocycloalkyl, and
(6) C₃₋₆ cycloalkyl,

wherein alkyl, phenyl, heteroaryl, heterocycloalkyl, and cycloalkyl are unsubstituted or substituted with one to three substituents independently selected from halogen, C₁₋₄ alkyl, hydroxy, and C₁₋₄ alkoxy, or two R⁶ substituents together with the atoms to which they are attached form a 4- to 8-membered mono- or bicyclic ring system optionally containing an additional heteroatom selected from O, S, —NH, and —NC₁₋₄ alkyl;

r is 1 or 2;

s is 0, 1, or 2;

n is 0, 1, 2, 3, or 4; and

p is 0, 1, or 2.

Similarly, WO 2006/020277 (Merck & Co, Inc) discloses a group of MC4 receptor agonists. However, their use in the treatment of lower urinary tract dysfunction is not mentioned. Thus, according to a further preferred aspect of the invention, the MC4 receptor agonist is a compound of formula (Id),

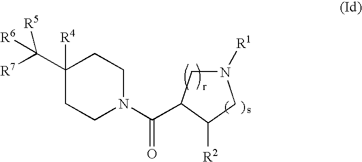

or a pharmaceutically acceptable salt thereof, wherein

R¹ is selected from the group consisting of:
(1 ) hydrogen,
(2) amidino,
(3) —C₁₋₄ alkyliminoyl,
(4) —C₁₋₈ alkyl,
(5) —(CH₂)ₙ—C₃₋₇ cycloalkyl,
(6) —(CH₂)ₙheterocycloalkyl,
(7) —(CH₂)ₙ-phenyl,
(8) —(CH₂)ₙ-naphthyl, and
(9) —(CH₂)ₙ-heteroaryl,

wherein phenyl, naphthyl, and heteroaryl are unsubstituted or substituted with one to three substituents independently selected from R³, and alkyl, cycloalkyl, and heterocycloalkyl are unsubstituted or substituted with one to three substitutents independently selected from R³ and oxo;

R² is selected from the group consisting of:
(1) phenyl,
(2) naphthyl, and
(3) heteroaryl,

wherein phenyl, naphthyl, and heteroaryl are unsubstituted or substituted with one to three substitutuents independently selected from R⁹;

each R³ is independently selected from the group consisting of:
(1) —C₁₋₈ alkyl,
(2) —(CH₂)ₙ-phenyl,
(3) —(CH₂)ₙ-heteroaryl,
(4) —(CH₂)ₙheterocycloalkyl,
(5) —(CH₂)ₙC₃₋₇ cycloalkyl,
(6) halogen,
(7) —OR⁸,
(8) —(CH₂)ₙC≡N,
(9) —(CH₂)ₙN(R⁸)₂,
(10) —(CH₂)ₙC(O)N(R⁸)₂,
(11) —(CH₂)ₙC(O)NR⁸N(R⁸)₂,
(12) —(CH₂)ₙC(O)NR⁸NR⁸C(O)R⁸, and
(13) —(CH₂)ₙCF₃,

wherein phenyl and heteroaryl are unsubstituted or substituted with one to three substituents independently selected from halogen, hydroxy, C₁₋₄ alkyl, trifluoromethyl, and C₁₋₄ alkoxy, and wherein any alkyl, cycloalkyl, heterocycloalkyl, and methylene (CH₂) carbon atom in R³ is unsubstituted or substituted with one to two substituents independently selected from halogen, hydroxy, oxo, C₁₋₄ alkyl, trifluoromethyl, and C₁₋₄ alkoxy, or two R³ substituents on the same carbon atom are taken together with the carbon atom to form a cyclopropyl group;

R⁴ is selected from the group consisting of:
(1) hydrogen, and
(2) —C₁₋₆ alkyl,
(3) —OC₁₋₆ alkyl, and
(4) —(CH₂)ₙN(R⁸)C(O)R⁸;

R⁵ is selected from the group consisting of:
(1) —CF₃,
(2) —C₁₋₆ alkyl,
(3) —C₂₋₈ alkenyl,
(4) —C₂₋₈ alkynyl,
(5) —OC₁₋₈ alkyl,
(6) —(CH₂)ₙC₃₋₇cycloalkyl,
(7) —(CH₂)ₙheterocycloalkyl,
(8) —(CH₂)ₙ-phenyl,
(9) —(CH₂)ₙ-naphthyl,
(10) —(CH₂)ₙheteroaryl, and
(11) —(CH₂)ₙC₃₋₇bicycloalkyl,

wherein phenyl, naphthyl, and heteroaryl are unsubstituted or substituted with one to three substituents independently selected from R³, and alkyl, alkenyl, alkynyl, cycloalkyl, heterocycloalkyl, and bicycloalkyl are unsubstituted or substituted with one to three substituents independently selected from R³ and oxo, and wherein any methylene (CH₂) in R⁵ is unsubstituted or substituted with one to two substituents independently selected from halogen, hydroxy, oxo, and C₁₋₄ alkyl;

R⁶ is selected from the group consisting of:
(1) hydrogen,
(2) —C₁₋₆ alkyl, and
(3) —OC₁₋₆ alkyl;

R⁷ is selected from the group consisting of:
(1) —(CH₂)ₙN(R⁸)₂,
(2) —(CH₂)ₙNR⁸C(O)R⁸,
(3) —(CH₂)ₙOR⁸,
(4) —(CH₂)ₙC≡N,
(5) —(CH₂)ₙC(O)OR⁸,
(6) —(CH₂)ₙC(O)N(R⁸)₂,
(7) —(CH₂)ₙNR⁸C(O)N(R⁸)₂,
(8) —(CH₂)ₙNR⁸C(O)heteroaryl,
(9) —(CH₂)ₙheteroaryl,
(10) —(CH₂)ₙNR⁸S(O)ₚR⁸,
(11) —(CH₂)ₙSR⁸, and
(12) —(CH₂)ₙS(O)ₚR⁸,

wherein heteroaryl is unsubstituted or substituted with one to three substituents selected from C₁₋₄ alkyl; and any methylene (CH₂) in R⁷ is unsubstituted or substituted with one to two substituents independently selected from halogen, hydroxyl, oxo, and C₁₋₄ alkyl, or two C₁₋₄ alkyl substituents on any methylene (CH₂) in R⁷ together with the atom to which they are attached form a 3, 4, 5, or 6-membered ring optionally containing an additional heteroatom selected from O, S, —NH, and —NC₁₋₄ alkyl;

each R⁸ is independently selected from the group consisting of:
(1) hydrogen,
(2) —C₁₋₈ alkyl,
(3) —C₂₋₈ alkenyl,
(4) —(CH₂)ₙC₃₋₇ cycloalkyl,
(5) —(CH₂)ₙheterocycloalkyl.
(6) —(CH₂)ₙ-phenyl, and
(7) —(CH₂)ₙ-heteroaryl;

each R⁹ is independently selected from the group consisting of:
(1) —C₁₋₈ alkyl,
(2) —C₂₋₈ alkenyl,
(3) —(CH₂)ₙ-phenyl,
(4) —(CH₂)ₙ-naphthyl,
(5) —(CH₂)ₙ-heteroaryl,
(6) —(CH₂)ₙheterocycloalkyl,
(7) —(CH₂)ₙC₃₋₇ cycloalkyl,
(8) halogen,
(9) —OR⁸,
(10) —(CH₂)ₙC(O)R⁸,
(11) —(CH₂)ₙOC(O)R⁸,
(12) —(CH₂)ₙC(O)OR⁸,
(13) —(CH₂)ₙC≡N,
(14) NO₂,
(15) —(CH₂)ₙN(R⁸)₂,
(16) —(CH₂)ₙC(O)N(R⁸)2,
(17) —(CH₂)ₙNR⁸C(O)R⁸,
(18) —(CH₂)ₙNR⁸C(O)OR⁸,
(19) —(CH₂)ₙNR⁸C(O)-heteroaryl,
(20) —(CH₂)ₙNR⁸C(O)N(R⁸)₂,
(21) —(CH₂)ₙC(O)NR⁸N(R⁸)₂,
(22) —(CH₂)ₙC(O)NR⁸NR⁸C(O)R⁸,
(23) —(CH₂)ₙNR⁸S(O)ₚR⁸,
(24) —(CH₂)ₙS(O)ₚN(R⁸)₂,
(25) —(CH₂)ₙS(O)ₚR⁸,
(26) —O(CH₂)ₙC(O)N(R⁸)₂,
(27) —(CH₂)ₙCF₃, and
(28) —O(CH₂)ₙCF₃,

wherein alkenyl, phenyl, naphthyl, and heteroaryl are unsubstituted or substituted with one to three substituents independently selected from halogen, hydroxy, C₁₋₄ alkyl, trifluoromethyl, and C₁₋₄ alkoxy, and wherein alkyl, cycloalkyl, heterocycloalkyl, and any methylene (CH₂) carbon atom in R⁹ are unsubstituted or substituted with one or two substituents independently selected from halogen, hydroxy, oxo, C₁₋₄ alkyl, trifluoromethyl, and C₁₋₄ alkoxy, or two R⁹ substituents on the same carbon atom are taken together with the carbon atom to form a cyclopropyl group;

r is 1 or 2;

s is 0, 1 or 2;

n is 0, 1, 2, 3, or 4; and

p is 0, 1, or 2.

Preferably, the lower urinary tract dysfunction is selected from:
(i) urinary incontinence (any condition in which there is an involuntary leakage of urine), including stress urinary incontinence, urge urinary incontinence and mixed urinary incontinence;
(ii) overactive bladder (OAB), which includes one or more of the symptoms of increased daytime frequency and urgency, and nocturia, which symptoms may or may not result in loss of urine (OAB wet and OAB dry), and urge incontinence; and
(iii) lower urinary tract symptoms (LUTS) comprising one or more of the above symptoms, and, when associated with BPH, at least one of the additional symptoms of terminal dribble, hesitancy, intermittency, straining and poor flow.

Preferably, the lower urinary tract dysfunction is urinary incontinence, more preferably it is stress urinary incontinence.

The MC4 receptor agonist can be used alone, or in combination with other agents, for the treatment of lower urinary tract dysfunction. The other agents include but are not limited to:
Muscarinic acetylcholine receptor antagonist such as tolterodine and fesoterodine
Alpha adrenergic receptor antagonist, in particular an alpha1 adrenergic receptor antagonist or an alpha2 adrenergic receptor antagonist
Alpha adrenergic receptor agonist or partial agonist, in particular an alpha1 adrenergic receptor agonist or partial agonist, or an alpha2 adrenergic receptor agonist or partial agonist
Serotonin and Noradrenalin reuptake inhibitor (SNRI)
Noradrenalin reuptake inhibitor (NRI) such as reboxetine
5HT2C agonist (see WO 04/096196)
Vanilloid receptor (VR) antagonist, such as capsaicin
alpha2delta ligand, such as gabapentin or pregabalin
PDE5 inhibitors, such as sildenafil, tadalafil, vardenafil and 5-[2-ethoxy-5-(4-ethyl-piperazine-1-sulphonyl)-pyridin-3-yl]-3-ethyl-2-[2-methoxy-ethyl]-2,6-dihydro-pyrazolo[4,3-d]pyrimidin-7-one (see WO 01/27113)
Beta 3 adrenergic receptor agonist or partial agonist such as YM-178
NK1 antagonist such as casopitant.

Therefore, pharmaceutical compositions of an MC4 receptor agonist compound with one or more of the other agents listed above are also included in the invention, as is their use in the treatment of lower urinary tract dysfunction. Also included in the present invention are products containing an MC4 receptor agonist as described herein, and an agent selected from the above list, as a combined preparation for simultaneous, separate or sequential use in the treatment of lower urinary tract dysfunction.

Preferably, the MC4 receptor agonist compound is able to penetrate into the human central nervous system (CNS). Thus, according to a broader aspect, the present invention further provides the use of an MC4 receptor agonist compound for the manufacture of a medicament for the treatment of lower urinary tract dysfunction, wherein the compound is able to penetrate into the human central nervous system (CNS).

Compounds having suitable CNS-penetrating ability are those for which at least 20% by weight of a given dose crosses the blood-brain barrier.

CNS-penetrating compounds generally have one or more of the following characteristics:
a molecular weight less than 450;
a polar surface area (PSA) of less than 90 Å²;
a log D between 1 and 3; and
a pKa between 7.5 and 10.5.

Polar surface area is defined as a sum of surfaces of polar atoms (usually oxygens, nitrogens and attached hydrogens) in a molecule. The calculation of PSA in a classical way is time consuming, because of the necessity to generate a reasonable 3D molecular geometry and then determine the surface itself. Alternatively, a different method, topological polar surface area (TPSA) is used.

The methodology for the calculation of TPSA is described in detail by Ertl, et al in ‘Fast calculation of molecular polar surface area as a sum of fragment based contributions and its application to the prediction of drug transport properties’, J. Med. Chem. 2000, 43: 3714-3717. Briefly, the procedure is based on the summation of tabulated surface contributions of polar fragments. Topological polar surface area provides results of practically the same quality as the classical 3D PSA.

Log D is a partition coefficient (log P) at pH 7.4. A partition coefficient is a measure of how a substance partitions between a lipid (here, octanol) and water, and hence of its lipophilicity. See for example Levin, J Med Chem, 1980, 23, 682-684.

pKa or dissociation constant is a measure of the strength of an acid or a base. The term is well known to those skilled in the art.

Dosages and Formulations

Pharmaceutical compositions suitable for the delivery of compounds used in the present invention and methods for their preparation will be readily apparent to those skilled in the art. Such compositions and methods for their preparation may be found, for example, in Remington's Pharmaceutical Sciences, 19th Edition (Mack Publishing Company, 1995).

Any suitable route of administration may be employed for providing a mammal, especially a human, with an effective dosage of an MC4 receptor agonist compound. For example, oral (including buccal and sublingual administration), rectal, topical, parental, ocular, pulmonary, nasal, and the like may be employed. Dosage forms include tablets, troches, dispersions, suspensions, solutions, capsules, creams, ointments, aerosols, and the like. Preferably, the compounds are administered orally.

The effective dosage of active ingredient employed may vary depending on the particular compound employed, the mode of administration, the condition being treated and the severity of the condition being treated. Such dosage may be ascertained readily by a person skilled in the art.

For the treatment of lower urinary tract dysfunction, MC4 receptor agonist compounds are given in a dose range of from about 0.001 milligram (mg) to about 1000 mg, preferably from about 0.001 mg to about 500 mg, more preferably from about 0.001 mg to about 100 mg, even more preferably from about 0.001 mg to about 50 mg and especially from about 0.002 mg to about 25 mg per kilogram of body weight, preferably as a single dose orally or as a nasal spray. For example, oral administration may require a total daily dose of from about 0.1 mg up to about 1000 mg, while an intravenous dose may only require from about 0.001 mg up to about 100 mg. The total daily dose may be administered in single or divided doses and may, at the physician's discretion, fall outside of the typical range given herein.

These dosages are based on an average human subject having a weight of about 65 kg to 70 kg. The physician will readily be able to determine doses for subjects whose weight falls outside this range, such as infants and the elderly.

The compounds of the invention may be administered orally. Oral administration may involve swallowing, so that the compound enters the gastrointestinal tract, and/or buccal, lingual or sublingual administration by which the compound enters the blood stream directly from the mouth.

Formulations suitable for oral administration include solid, semi-solid and liquid systems such as tablets; soft or hard capsules containing multi- or nano-particulates, liquids, or powders; lozenges (including liquid-filled); chews; gels; fast dispersing dosage forms; films; ovules; sprays; and buccal/mucoadhesive patches.

Liquid formulations include suspensions, solutions, syrups and elixirs. Such formulations may be employed as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet. may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986 by Liang and Chen (2001).

For tablet dosage forms, depending on dose, the drug may make up from 1 wt % to 80 wt % of the dosage form, more typically from 5 wt % to 60 wt % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 wt % to 25 wt %, preferably from 5 wt % to 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 wt % to 5 wt % of the tablet, and glidants may comprise from 0.2 wt % to 1 wt % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 wt % to 10 wt %, preferably from 0.5 wt % to 3 wt % of the tablet.

Other possible ingredients include anti-oxidants, colourants, flavouring agents, preservatives and taste-masking agents.

Exemplary tablets contain up to about 80% drug, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet blends may be compressed directly or by roller to form tablets. Tablet blends or portions of blends may alternatively be wet-, dry-, or melt-granulated, melt congealed, or extruded before tablefting. The final formulation may comprise one or more layers and may be coated or uncoated; it may even be encapsulated.

The formulation of tablets is discussed in Pharmaceutical Dosage Forms: Tablets, Vol. 1, by H. Lieberman and L. Lachman (Marcel Dekker, New York, 1980).

Consumable oral films for human or veterinary use are typically pliable water-soluble or water-swellable thin film dosage forms which may be rapidly dissolving or mucoadhesive and typically comprise a compound of formula I, a film-forming polymer, a binder, a solvent, a humectant, a plasticiser, a stabiliser or emulsifier, a viscosity-modifying agent and a solvent. Some components of the formulation may perform more than one function.

The MC4 receptor agonist compound may be water-soluble or insoluble. A water-soluble compound typically comprises from 1 weight % to 80 weight %, more typically from 20 weight % to 50 weight %, of the solutes. Less soluble compounds may comprise a greater proportion of the composition, typically up to 88 weight % of the solutes. Alternatively, the MC4 receptor agonist compound may be in the form of multiparticulate beads.

The film-forming polymer may be selected from natural polysaccharides, proteins, or synthetic hydrocolloids and is typically present in the range 0.01 to 99 weight %, more typically in the range 30 to 80 weight %.

Other possible ingredients include anti-oxidants, colorants, flavourings and flavour enhancers, preservatives, salivary stimulating agents, cooling agents, co-solvents (including oils), emollients, bulking agents, anti-foaming agents, surfactants and taste-masking agents.

Films are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Pharmaceutical Technology On-line, 25(2), 1-14 by Verma et al (2001). The use of chewing gum to achieve controlled release is described in WO 00/35298.

The MC4 receptor agonist compound may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular, intrasynovial and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of MC4 receptor agonist compounds used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus compounds of the invention may be formulated as a suspension or as a solid, semi-solid, or thixotropic liquid for administration as an implanted depot providing modified release of the active compound. Examples of such formulations include drug-coated stents and semi-solids and suspensions comprising drug-loaded poly(dl-lactic-coglycolic)acid (PGLA) microspheres.

The MC4 receptor agonist compounds may also be administered topically, (intra)dermally, or transdermally to the skin or mucosa. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958 by Finnin and Morgan (October 1999).

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release.

The MC4 receptor agonist compounds can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane, or as nasal drops. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose, blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise an MC4 receptor agonist compound, propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations intended for inhaled/intranasal administration.

Formulations for inhaled/intranasal administration may be formulated to be immediate and/or modified release using, for example, PGLA. Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” containing from 0.001 mg to 10 mg of the MC4 receptor agonist compound. The overall daily dose will typically be in the range 0.001 mg to 40 mg which may be administered in a single dose or, more usually, as divided doses throughout the day.

The MC4 receptor agonist compound may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

The MC4 receptor agonist compound may also be administered directly to the eye or ear, typically in the form of drops of a micronised suspension or solution in isotonic, pH-adjusted, sterile saline. Other formulations suitable for ocular and aural administration include ointments, gels, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted, or programmed release.

The MC4 receptor agonist compound may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

Inasmuch as it may desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for coadministration of the compositions.

For the avoidance of doubt, references herein to “treatment” include references to curative, palliative and prophylactic treatment.

Biological Assays

The MC4 receptor agonist utilized in the present invention is preferably selective for the MC4 receptor over other MC receptor subtypes. Methods for determining receptor subtype selectivity are well known to those skilled in the art, and have been described for MC receptor subtypes by Palucki et al, Bioorganic & Medicinal Chemistry Letters, vol 15, issue 1, 3 January 2005, pages 171-175. Preferably, the MC4 receptor agonist has a binding affinity for MC4 receptors that is greater than, preferably 10 times greater than, more preferably 100 times greater than its binding affinity for MC3 and/or MC5 receptors.

According to the present invention, MC4 receptor agonists, in particular the compounds of formula I, Ia, Ib and Id are useful in the treatment of conditions of lower urinary tract dysfunction including but not exclusively restricted to overactive bladder, increased daytime frequency, nocturia, urgency, urinary incontinence (any condition in which there is an involuntary leakage of urine), including stress urinary incontinence, urge urinary incontinence and mixed urinary incontinence, overactive bladder with associated urinary incontinence, enuresis, nocturnal enuresis, continuous urinary incontinence, situational urinary incontinence such as incontinence during sexual intercourse, and lower urinary tract symptoms associated with benign prostatic hyperplasia (LUTS associated with BPH). Activity of such compounds on lower urinary tract function, and thus their potential usefulness in treating conditions involving lower urinary tract dysfunction, can be investigated and assessed utilising a number of standard in vivo models known to those skilled in the art and frequently described in the literature (Morrison, J., et al., Neurophysiology and Neuropharmacology. In: Incontinence, Ed. Abrams, P., Cardozo, C., Khoury, S. and Wein, A. Report of the World Health Organisation Consensus Conference. Paris, France: Health Publications Ltd., 2002: 83-163; Brune M E et al. J Urol. 2001, 166:1555-9; WO 2005/010534). As an example compounds used in the present invention can be tested for such effects in the following models:
Investigation of bladder capacity and external urethral sphincter (EUS) function in the guinea-pig:

Experiments are performed in adult female guinea pigs, weighing approx 500 g. All animals are initially anaesthetised with halothane (4%), carried in oxygen (3-4 L min⁻¹) and maintained at an appropriate surgical plane with urethane (25% w/v; 0.5 ml 100 g⁻¹ body weight). The trachea, a jugular vein and a carotid artery are cannulated for respiratory ventilation, injection of test compound and monitoring of blood pressure, respectively. A midline laporatomy is performed to expose the urinary bladder and a cystometry tube inserted through a small incision in the dome of the bladder and secured in place. The abdominal wound is then closed tightly around the externalised cystometry tube, which, in turn, is connected to an infusion pump and pressure transducer, for filling the bladder and recording intravesical pressure, respectively. Electromyographic (EMG) wire leads are inserted into the EUS striated muscle layer opposed to the dorsal surface of the symphysis pubis. The EMG leads are connected to an appropriate amplification and electrical filter system and changes in EUS electrical activity displayed on an oscilloscope and recorded through appropriate computer software.

Following a 30 min post surgery stabilisation period, the bladder is filled at a rate of 150 μl min⁻¹ with physiological saline (room temperature), until initiation of a micturition reflex is observed. Following micturition, the bladder is drained via the externalised cystometry tube. Bladder filling is then repeated at least 3 times (or until repeatable filling cycles are achieved) in order to establish a mean bladder threshold capacity for initiation of micturition. EUS EMG activity and intravesical (bladder) pressure are recorded throughout bladder filling. Subsequently, test compound or vehicle is injected intravenously utilising either a bolus dose or constant infusion and bladder filling re-initiated (150 μl min⁻¹) until micturition occurs, the bladder is then drained as before and the process repeated with addition of increasing doses of test compound (2 micturition responses are measured at each compound concentration). Changes in threshold bladder capacity initiating micturition and/or in EUS EMG activity are indicative of compound activity on lower urinary tract function.
Investigation of abdominal leak point pressure in the guinea-pig:

Experiments are performed in adult female guinea pigs, weighing approx 500 g. All animals are initially anaesthetised with halothane (4%), carried in oxygen (3-4 L min⁻¹) and maintained at an appropriate surgical plane with urethane (25% w/v; 0.5 ml 100 g⁻¹ body weight). The trachea, a jugular vein and a carotid artery are cannulated for respiratory ventilation, injection of test compound and monitoring of blood pressure, respectively. A midline laporatomy is performed to expose the urinary bladder and a cystometry tube inserted through a small incision in the dome of the bladder and secured in place. The abdominal wound is then closed tightly around the externalised cystometry tube, which, in turn, is connected to an infusion pump and pressure transducer, for filling the bladder and recording intravesical pressure, respectively. Electromyographic (EMG) wire leads are inserted into the EUS striated muscle layer opposed to the dorsal surface of the symphysis pubis. The EMG leads are connected to an appropriate amplification and electrical filter system and changes in EUS electrical activity displayed on an oscilloscope and recorded through appropriate computer software.

Following a 30 min post surgery stabilisation period, the bladder is filled at a rate of 150 μl min⁻¹ with physiological saline (room temperature), until initiation of a micturition reflex is observed. Following micturition, the bladder is drained via the externalised cystometry tube. Bladder filling is then repeated at least 3 times (or until repeatable filling cycles are achieved) in order to establish a mean bladder threshold capacity for initiation of micturition. EUS EMG activity and intravesical (bladder) pressure are recorded throughout bladder filling. Subsequently, the bladder is filled (150 μl min⁻¹) to 75% of this threshold volume with physiological saline and, through the use of a specially constructed frame, increasing weight is applied to the ventral surface of the abdomen of the animal just rostral to the position of the bladder until leakage of fluid is observed at the urethral meatus. This process is repeated at least 3 times in order to establish control responses; EUS EMG activity and intravesical pressure being recorded throughout. Subsequently increasing concentrations of test compound or vehicle is injected intravenously utilising either a bolus dose or constant infusion and weight induced leak responses re-investigated at each concentration. Changes in the abdominal weight required to induce leak and/or the maximum EUS EMG activity recorded immediately prior to leak are indicative of compound activity on lower urinary tract function.
Investigation of guinea-pig urethral pressure profilometry:

Experiments are performed in adult female guinea pigs, weighing approx 500 g. All animals are initially anaesthetised with halothane (4%), carried in oxygen (3-4 L min⁻¹) and maintained at an appropriate surgical plane with urethane (25% w/v; 0.5 ml 100 g⁻¹ body weight). The trachea, a jugular vein and a carotid artery are cannulated for respiratory ventilation, injection of test compound and monitoring of blood pressure, respectively. A midline laporatomy is performed to expose the urinary bladder and a cystometry tube inserted through a small incision in the dome of the bladder and secured in place. The abdominal wound is then closed tightly around the externalised cystometry tube, which, in turn, is connected to an infusion pump and pressure transducer, for filling the bladder and recording intravesical pressure, respectively. Electromyographic (EMG) wire leads are inserted into the EUS striated muscle layer opposed to the dorsal surface of the symphysis pubis. The EMG leads are connected to an appropriate amplification and electrical filter system and changes in EUS electrical activity displayed on an oscilloscope and recorded through appropriate computer software.

Following a 30 min post surgery stabilisation period, the bladder is filled at a rate of 150 μl min⁻¹ with physiological saline (room temperature), until initiation of a micturition reflex is observed. Following micturition, the bladder is drained via the externalised cystometry tube. Bladder filling is then repeated at least 3 times (or until repeatable filling cycles are achieved) in order to establish a mean bladder threshold capacity for initiation of micturition. Subsequently, the bladder is filled (150 μl min⁻¹) to 75% of this threshold volume and urethral tone (peak urethral pressure (PUP), functional urethral length (FUL) and closing pressure (CP)) assessed with the aid of a 3F Millar pressure transducer (Millar Instruments, Texas, US) inserted into the bladder through the external meatus. The urethral Millar pressure transducer is then retracted along the length of the urethra (urethral pull through) at a rate of 1 cm/min enabling the determination of PUP, FUL and CP. Urethral pull throughs are repeated every 2 min until 4 reproducible urethral profiles are observed. Subsequently increasing concentrations of test compound or vehicle is injected intravenously utilising either a bolus dose or constant infusion and a further 4 urethral pull throughs carried out at each concentration investigated. Changes in the PUP, FUL, CP or EUS EMG activity are indicative of compound activity on lower urinary tract function.
Investigation of dog urethral pressure profilometry (Test A):

Female beagle dogs (10-15 kg) are anaesthetised with sodium pentobarbitone (60 mg/mL solution) administered intravenously (IV) at 0.5 ml/kg via the right cephalic vein. Immediately following induction of anaesthesia the dog is intubated and respiration supported by artificial ventilation with oxygen. End tidal CO₂ is monitored continuously, using a Datex CO₂/O₂ monitor and maintained between 4.5 and 4.8% and body temperature maintained between 37° C. and 38° C. An incision is made in the right medial thigh and a polyethylene catheter (6F) inserted into the right femoral vein for administration of compounds and fluid maintenance; immediately venous access is achieved a bolus IV dose of α-chloralose (1% w/v) is administered at 35 mg/kg. A polyethylene catheter (4F) is inserted into the right femoral artery for blood sampling. An incision is made in the right foreleg and the brachial vein and artery isolated, maintenance of anaesthesia is achieved with α-chloralose/borax administered IV at the rate of 10 mg/kg/h via a polyethylene catheter (6F) inserted into the right brachial vein. A laparotomy is performed from the umbilicus to the top of the pubic symphysis via the midline to expose the peritoneum in order to expose the bladder. Both ureters are cannulated towards the kidneys with polyethylene catheters (6F) and urine collected externally; the bladder is catheterised through the dome with a polyethylene catheter (6F), which is in turn connected to a pressure transducer. In order to maintain constant bladder pressure at 10-15 mmHg, urine is removed and ambient temperature saline infused into the bladder. Immediately following the completion of the surgical procedures a further bolus dose of α-chloralose/borax solution is administered IV at 35 mg/kg and the animal allowed to stabilise for a period period ca. 1 hr, during which time haemodynamic and urological parameters were monitored.

Urethral tone (peak urethral pressure (PUP), functional urethral length (FUL) and closing pressure (CP)) is assessed with the aid of an 8F Millar pressure transducer (Millar Instruments, Texas, US) inserted into the bladder through the external meatus. The urethral Millar pressure transducer is then retracted along the length of the urethra (urethral pull through) at a rate of 1 cm/min enabling the determination of PUP, FUL and CP. Urethral pull throughs are repeated every 6 min until at least 4 reproducible urethral profiles are observed. Subsequently increasing concentrations of test compound or vehicle is injected intravenously utilising either a bolus dose or constant infusion and at least a further 4 urethral pull throughs carried out at each concentration investigated. Changes in the PUP, FUL or CP are indicative of compound activity on lower urinary tract function.
Investigation of bladder capacity and external urethral sphincter (EUS) function in the spontaneously hypertensive rat:

Experiments are performed in adult female spontaneously hypertensive rats (SHRs), weighing approx 250-300 g. All animals are initially anaesthetised with isoflurane (4%), carried in oxygen (3-4 L min⁻¹) and maintained at an appropriate surgical plane with urethane (25% w/v; 0.5 ml 100 g⁻¹ body weight). The trachea, a jugular vein and a carotid artery are cannulated for respiratory ventilation, injection of test compound and monitoring of blood pressure, respectively. A midline laporatomy is performed to expose the urinary bladder and a cystometry tube inserted through a small incision in the dome of the bladder and secured in place. The abdominal wound is then closed tightly around the externalised cystometry tube, which, in turn, is connected to an infusion pump and pressure transducer, for filling the bladder and recording intravesical pressure, respectively. Electromyographic (EMG) wire leads are inserted into the EUS striated muscle layer opposed to the dorsal surface of the symphysis pubis. The EMG leads are connected to an appropriate amplification and electrical filter system and changes in EUS electrical activity displayed on an oscilloscope and recorded through appropriate computer software.

Following a 30 min post surgery stabilisation period, the bladder is filled at a rate of between 45 and 100 μl min⁻¹ with physiological saline (room temperature), until initiation of a micturition reflex is observed. Following micturition, the bladder is drained via the externalised cystometry tube. Bladder filling is then repeated at least 3 times (or until repeatable filling cycles are achieved) in order to establish a mean bladder threshold capacity for initiation of micturition. EUS EMG activity and intravesical (bladder) pressure are recorded throughout bladder filling. Subsequently, test compound or vehicle is injected intravenously utilising either a bolus dose or constant infusion and bladder filling re-initiated until micturition occurs, the bladder is then drained as before and the process repeated with addition of increasing doses of test compound (2 micturition responses are measured at each compound concentration). Changes in threshold bladder capacity initiating micturition and/or in EUS EMG activity are indicative of compound activity on lower urinary tract function.
Investigation of voided volume in conscious ovariectomised mice:

Ovariectomised adult female mice are dosed (either orally or sub-cutaneously) with vehicle or increasing concentrations of compound and placed in individual metaboles with free access to water for 3 hr. Urine voided by each mouse is captured on a conical sponge within a container placed beneath each metabole, this sponge also deflects faecal pellets. The total volume of urine voided within the 3 hr period and the volume of urine per void is measured by a balance placed directly beneath the collection container. The average volume of urine per void and the frequency of voiding events are compared between vehicle and compound treated groups (up to n=16 per group), changes in these parameters in the absence of changes in the total urine output are indicative of compound activity on lower urinary tract function.
Investigation of voided volume and bladder activity in conscious telemeterised spontaneously hypertensive rat

Adult female spontaneously hypertensive rats are dosed (either orally or sub-cutaneously) with vehicle or increasing concentrations of compound and placed in individual metaboles with free access to water for 3 hr. Urine voided by each rat is captured on a conical sponge within a container placed beneath each metabole, this sponge also deflects faecal pellets. The total volume of urine voided within the 3 hr period and the volume of urine per void is measured by a balance placed directly beneath the collection container. The average volume of urine per void and the frequency of voiding events are compared between vehicle and compound treated groups (up to n=16 per group), changes in these parameters in the absence of changes in the total urine output are indicative of compound activity on lower urinary tract function.

MC4 Receptor Functional Assay

Assay Concept

Determination of compound activity against the human MCR4 receptor subtype was carried out using an immortalised CHO—K1 cell line that had been engineered to stably express both the recombinant human MCR4 receptor and a β-lactamase gene reporter (CHO—K1-MC4R-CRE-β-lactamase). This cell line was engineered using protocols akin to those outlined by Zaccolo et al (Zaccolo, M., (2000) Nature, 2(1); 25-29).

Compound-induced activation of the MCR4 receptors in the cell line stimulates the production, and intracellular accumulation of, the enzyme β-lactamase. The quantity of β-lactamase enzyme produced is directly proportional to the degree to which the test compound activates the MCR4 receptors present on the cells and is quantified using the β-lactamase gene reporter analysis kit that is commercially available from Invitrogen Life Technologies. An in-depth description of this technology and assay protocols are available from the Invitrogen web site (www.invitrogen.com). The protocol listed below provides a summary of that assay methodology.

The quantity of β-lactamase enzyme produced by compound-induced activation of the MCR4 receptors expressed in the cell line was quantified using a Ljl Biosystems Analyst™ HT 96.384 plate reader set to excite at a wave length of 405 nm, and measure the energy emitted at wave lengths of 450 nm and 530 nm. Cellular responses were quantified by dividing the measured energy emitted at a wavelength of 450 nm by the measured energy emitted at a wavelength of 530 nm. Data analysis was subsequently performed using a curve-fitting program and the apparent potency of the test compound (expressed as an EC₅₀ and defined as the effective compound concentration that elicited 50% of the maximum compound-induced response) extrapolated from the fitted curve.

Materials

From Invitrogen: Dulbecco's modified Eagle media (DMEM) with Glutamax-1, Cat N^{o} 32430-027; Non-essential amino acids, Cat N^{o} 1140-0.35; Geneticin (G418), Cat N^{o} 10131-027; Cell dissociation buffer (enzyme-free PBS-based), Cat N^{o} 13151-014; Phosphate buffered saline (PBS) (w/o Ca²⁺ and Mg²⁺), Cat N^{o} 14190-094; CCF4-AM, Cat N^{o} K1028; Pluronic F127s solution (Solution B), Cat N^{o} K1026N; 24% PEG and 18% TR40 solution (Solution C), Cat N^{o} K1026N; Zeocin, Cat N^{o} R250-05.

From Sigma: Foetal calf serum (FCS), Cat N^{o} F7524; Sodium pyruvate, Cat N^{o} S8636; N-(2-Hydroxyethyl)piperazine-N′-(2-ethanesulfonic acid) (HEPES), Cat N^{o} H0887; Dimethyl sulphoxide (DMSO), Cat N^{o} D-8418; Cyclohexamide, Cat N^{o} C-7698; Trypan blue solution, Cat N^{o} T-4424; Probenecid, Cat N^{o} P8761; Bovine serum albumin (BSA), Cat N^{o} A2153; Pluronic F-127, Cat N^{o} 9003-11-6.

From Gilson: pipettes ranging from 10 μl to 1000 μl.

From Hereaus; Hera Cell CO₂ cell incubator.

From Medical Air Technology; BioMat² Class II Microbiological safety cabinet

From Ljl Biosystems; Analys™ HT 96.384 plate reader set to excite at a wavelength of 405 nm, and measure the energy emitted at wavelengths of 450 nm and 530 nm.

From Bachem: α-Melanocyte Stimulating Hormone α-MSH, Cat N^{o} H1075, used as a positive control compound.

Buffers

CCF4-AM was dissolved in 100% DMSO to give a final solution concentration of 1 mM. This solution was termed Solution A.

Probenecid was dissolved in 200 mM NaOH to give a final solution concentration of 200 mM. This solution was termed Solution D.

Composition of the β-lactamase assay dye solution: for 1072 μL of assay dye solution combine: 12 μL of Solution A, 60 μL of Solution B, 925 μL of Solution C and 75 μL of Solution D.

Consumables

From Greiner: 384-well black μclear bottom Microplate assay plates, Cat No. 781091.

From Costar: Sterile Pipettes from 2 up to 50 ml volume, Sterile tips from P10 up to P1000; Sterile reservoirs, Cat No. 4878; T225 flasks vent cap, Cat No. 3001.

Compound Preparation

All test compounds were initially dissolved in DMSO to give a compound concentration of 4 mM and then further diluted for the assay in PBS, containing 2.5% v/v DMSO and 0.05% w/v pluronic F-127, to give actual concentrations 5-fold greater than that desired as the final assay concentration.

Day-To-Day Cell Culture

Cells were grown in T225 vent cap flasks containing 50 ml of growth medium and maintained in a cell incubator at a temperature of 37° C. and in an environment containing 5% CO₂. The composition of the growth medium for the CHO—K1-MC4R-CRE-β-lactamase was 90% v/v DMEM supplemented with; Glutamax-1, 25 mM HEPES, 10% v/v foetal calf serum (FCS), 1 mM sodium pyruvate, 0.1 mM non essential amino acids and 800 μg/ml Geneticin, further supplemented with 200 μg/ml Zeocin. Cells were harvested when they reached 80-90% confluency by first removing the existing growth medium and then washing with PBS that had been pre-warmed to a temperature of 37° C. This PBS was then removed and 5 ml of cell dissociation fluid added to the flask. These cells were incubated for 5 minutes in a cell incubator set at a temperature of 37° C. and in an environment containing 5% CO₂ to detach the cells. When cells were detached, pre-warmed growth media was added, the cells re-suspended and mixed gently to achieve a single cell suspension by pipetting. This cell suspension was then used for experimentation, or transferred into a new T225 flask to perpetuate the cell culture.

Assay Procedure

On the first day of the assay cells were harvested as described above. A suspension of cells at 2×10⁵ cells/ml in modified growth medium, containing 5% FCS, was prepared and 40 μl of this cell suspension added into each well of a Greiner 384-well black μclear bottom Microplate assay plate.

The cell plates were then retuned to a cell incubator maintained at a temperature of 37° C. and in an environment containing 5% CO₂ overnight before performing the assay on the second assay day.

On the second day of the assay the cell plate was removed from the cell incubator and 10 μL of the test compound solution was transferred to the assay plate. The assay plate was then transferred to a cell incubator, set at 37° C. and in an environment containing 5% CO₂, and left for 4 hours. After this incubation period the plate was removed from the incubator, 10 μL of the β-lactamase assay dye solution was added to each well and then the plate returned to the cell incubator. Following a further incubation period of 60 minutes the plates were removed from the incubator and transferred to the Ljl Biosystems Analyst™ HT 96.384 plate reader for quantification.

Compounds stimulating a statistically significant increase in β-lactamase enzyme levels (in comparison with control vehicle solution) in this functional assay are regarded as MC4 receptor agonists. Preferably, MC4 receptor agonist compounds used in the present invention are at least 50% agonists in comparison with the compound of Example 8 below (first disclosed in Provisional U.S. Patent Application 60/706,191, applicant's reference PC 33020, mentioned above). More preferably, they are full agonists in comparison with the compound of Example 8 below.

MC4 Receptor Binding Assay—AGRP Inhibition

Agouti related protein (AGRP) is a high affinity endogenous antagonist for the MC4 receptor (Lu et al., 1994, Nature 371: 799-802; Ollman et al., 1997, Science 278: 135-138). AGRP levels are upregulated by fasting (Mizuno & Mobbs 1999, Endocrinology. 140: 4551-4557) and therefore it is important to assess the ability of anti-obesity agents acting through the MC4 receptor to inhibit the binding of AGRP. It has been ascertained that this C-terminal fragment of AGRP contains the MC4R binding determinants (Yang et al., 1999, Mol Endocrinol 13: 148-155), therefore, compounds can be evaluated for their ability to inhibit AGRP binding to membranes from cells expressing the MC4R using a competition binding assay versus [¹²⁵I]AGRP(87-132). To this end cells expressing the MC4R were subject to homogenisation and the membrane fragment isolated by differential centrifugation. CHO—CRE MC4R cell membranes (12 μg protein) were incubated with 0.3 nM [¹²⁵I]AGRP(87-132) and 11 half-log concentrations of competitor ligand, in duplicate, in a total volume of 100 μl buffer (25 mM HEPES,1 mM MgCl₂, 2.5 mM CaCl₂, 0.5% BSA pH 7.0). Non-specific binding was determined by the inclusion of 1 μM SHU9119. The reaction was initiated by the addition of membranes and plates were incubated at room temperature for 2 hours. The reaction was terminated by rapid filtration onto GF/C filters (presoaked in 1% PEI) using a vacuum harvester followed by five 200 μl washes of ice cold wash buffer (Binding buffer containing 500 mM NaCl). The filters were soaked in 50 μl scintillation fluid and the amount of radioactivity present was determined by liquid scintillation counting. Ki values were determined by data analysis using appropriate software.

Preferably the compounds used in the present invention exhibit a binding constant at the MC4 receptor expressed as a Ki value against AGRP of lower than about 100 nM, more preferably lower than 20 nM.

### Preparation of Compounds Disclosed in U.S. Patent Application 60/706,191 (Applicant's Reference PC 33020)

The routes below illustrate methods of synthesising compounds of formula (Ia). The skilled person will appreciate that other methods may be equally as viable.

Scheme 1 illustrates the preparation of compounds of formula (Ia) via peptide coupling of intermediates (II) and (III), if necessary adding a suitable base and/or additive (such as 1-hydroxybenzotriazole hydrate or 4-dimethylaminopyridine).

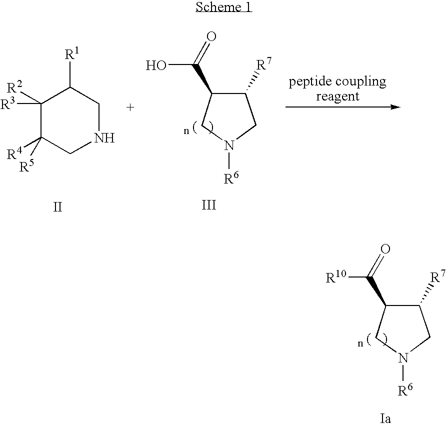

In respect of compounds (Ia), (II), (III) in Scheme 1 the definitions of R¹, R², R³, R⁴, R⁵, R⁶, R⁷ and R¹⁰ are as defined hereinbefore for compounds of formula (Ia) unless stated otherwise. Alternative conditions employed involve stirring a solution of the piperidine (amine) of general formula (II) and the pyrrolidine (acid) of general formula (III) together with 1-(3-dimethylaminopropyl)-3-ethyl-carbodiimide hydrochloride (EDCI), triethylamine or N-methylmorpholine and 1-hydroxybenzotriazole hydrate (HOBt) in dimethylformamide (DMF), tetrahydrofuran (THF), dichloromethane (DCM) or ethyl acetate at room temperature. An alternative suitable procedure is to stir a solution of the intermediate compounds of general formula (II) and general formula (III) together with O-benzotriazol-1-yl-N,N,N′,N′-tetramethyluronium hexafluorophosphate (HBTU) or 1-propylphosphonic acid cyclic anhydride in CH₂Cl₂ or EtOAc. Any suitable inert solvent may be used in place of those mentioned above, wherein inert solvent means a solvent which does not contain a carboxylic acid or primary or secondary amine. At least one equivalent of each of the coupling reagents should be used and an excess of either one or both may be used if desired.

Scheme 2 illustrates an alternative route for the preparation of compounds of general formula (Ia), having a range of R⁶ groups, via utility of a protecting group strategy.

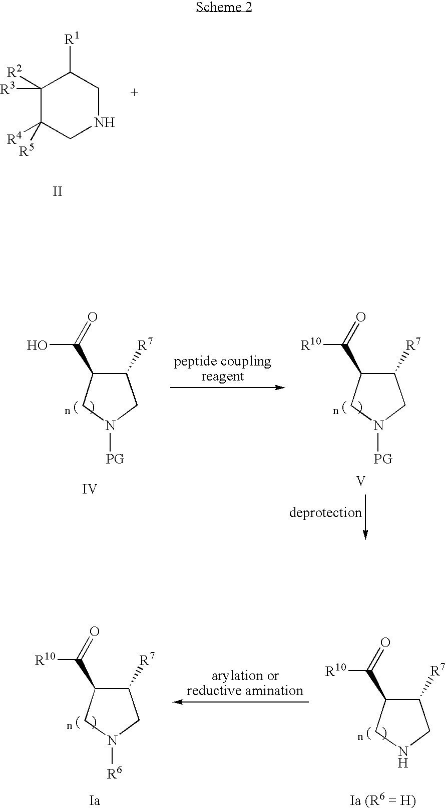

In respect of compounds (Ia), (II), (IV) and (V) in Scheme 2, the definitions of R¹, R², R³, R⁴, R⁵, R⁶, R⁷ and R¹⁰ are as defined hereinbefore for compounds of formula (Ia) unless stated otherwise. PG is a nitrogen-protecting group.

In scheme 2 the amine intermediates of general formula (II) and protected pyrrolidine acid intermediates of general formula (IV) are coupled using standard peptide coupling methods as previously described in scheme 1 to provide a coupled and protected intermediate of general formula (V) from which the nitrogen protecting group can be removed using standard de-protection strategies to furnish a compound of general formula (I) in which R⁶═H. Any suitable nitrogen protecting groups may be used (as described in “Protecting Groups in Organic Synthesis” 3^{rd} Edition T. W. Greene and P.G. Wuts, Wiley-Interscience, 1999). A common nitrogen protecting group (PG) suitable for use herein is tert-butoxycarbonyl, which is readily removed by treatment with an acid such as trifluoroacetic acid or hydrogen chloride in an organic solvent such as dichloromethane or 1,4-dioxane.

Alternative substituents such as alkyl and cycloalkyl groups may be introduced at R⁶ by using conventional alkylation techniques. Suitable methods for alkylation of secondary amines include:
(i) reaction with an aldehyde or ketone and a hydride reducing agent such as sodium triacetoxyborohydride, optionally in the presence of acetic acid, in an inert solvent such as dichloromethane or acetonitrile;
(ii) reaction with an alkyl halide or suitably activated alcohol derivative (e.g. as a sulfonate ester) in the presence of a base (such as triethylamine) in an inert solvent;

Aryl and heteroaryl groups may be introduced at R⁶ by displacement of a suitable leaving group from an aromatic precursor. Suitable leaving groups include halogens. In certain cases transition metal catalysis (e.g. palladium, copper), optionally in combination with a phosphine ligand such as 1,1′-binaphthalene-2,2′-diylbisdiphenylphosphine, may be required to achieve the required coupling products. Ketones and ester groups may be introduced at R⁶ by techniques that will be well-known to those skilled in the art by reference to literature precedents and the examples and preparations herein.

Scheme 3a illustrates the route for preparation of the pyrrolidine acid intermediates of general formula (III) from unsaturated ester intermediates of general formula (VI).

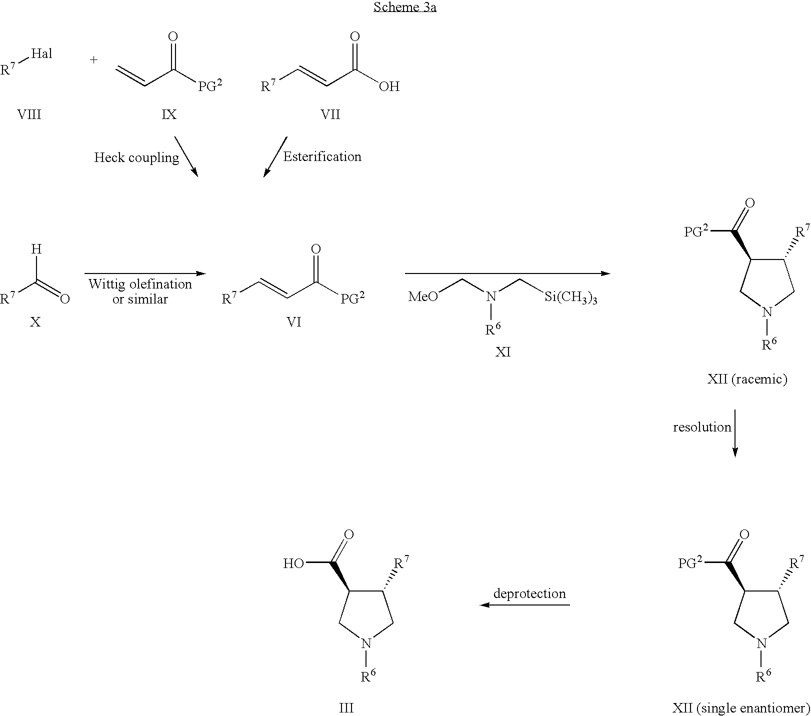

In respect of compounds (III), (VI), (VII), (VIII), (IX), (X), (XI), (XII) in scheme 3 the definitions of R⁶ and R⁷ are as defined hereinbefore for compounds of formula (I) unless stated otherwise. PG² is a suitable carboxylic acid protecting group. Compounds of formulae (VII), (VIII), (X) and (IX) are either commercially available or will be well-known to those skilled in the art with reference to literature precedents and/or the preparations herein.

Compounds of general formula (VI) can be made predominantly as the desired trans-isomer by Wittig or similar olefination of an aldehyde intermediate of general formula (X) with a suitable ylid e.g. methyl(triphenylphosphoranylidene)acetate, or a phosphonate anion e.g. derived from deprotonation of trimethylphosphonoacetate.

Many alternative methods exists in the literature for the production of unsaturated ester intermediates of general formula (VI), including esterification of a precursor cinnamic acid derivative (VII) using standard esterification methods, or Heck reaction of an aromatic halide (VIII) with a suitable acrylate derivative (IX), such as t-butyl acrylate, in the presence of a palladium catalyst and a suitable base, such as triethylamine.

The resulting E-olefin intermediate of general formula (VI) will undergo a [3+2]-azomethine ylid cycloaddition by reaction with an ylid precursor of general formula (XI), to provide a pyrrolidine with almost exclusively the trans-stereochemistry. This reaction requires an inert solvent such as dichloromethane or toluene or tetrahydrofuran and activation by one or more of: (1) an acid catalyst, such as TFA; (2) a desilylating agent such as silver fluoride; (3) heating.

The compound of general formula (XII) obtained from the cycloaddition reaction is a racemate and may require resolution into its constituent enantiomers, which can be achieved by preparative HPLC using a chiral stationary phase. Alternatively the acid intermediate of general formula (III) can be resolved by standard methods (e.g. formation of diastereomeric derivatives by reaction with an enantiomerically pure reagent, separation of the resulting diastereomers by physical methods and cleaving to acid (III).

Intermediate compounds of general formula (XII) can be converted into compounds of general formula (III) by deprotection of the protecting group. Many methods are available to achieve this transformation (see Advanced Organic Chemistry: Reactions, Mechanisms, and Structure, Fourth Edition. March, Jerry, 1992, pp 378-383 published by Wiley, New York, N.Y. USA). In particular, for base labile protecting groups, treatment of a compound of general formula (XII) with an aqueous alkali metal hydroxide solution, such as lithium hydroxide, sodium hydroxide or potassium hydroxide in a suitable organic solvent will provide the corresponding compounds of general formula (III). Preferably water-miscible organic co-solvents (such as 1,4-dioxane or tetrahydrofuran) are also utilised in such reactions. If required, the reaction may be heated to assist the hydrolysis. Deprotection of certain protecting groups may also be achieved using acid conditions e.g. by heating the protected derivative in an aqueous acid such as hydrochloric acid. Certain protecting groups are more conveniently hydrolysed in acidic conditions e.g. tert-butyl or benzhydryl esters. Such esters can be cleaved by treatment with anhydrous acids such as trifluoroacetic acid or hydrogen chloride in an inert organic solvent such as dichloromethane.

Scheme 3b illustrates an alternative route for the preparation of a single enantiomer of the pyrrolidine acid intermediate of general formula (III) from unsaturated intermediates of general formula (VI), using an oxazolidinone as a chiral auxiliary. The acid of formula (XIII) may be obtained by deprotection of (VI) and then coupled to an oxazolidinone (where R is preferably phenyl, tertiary butyl, or iso-propyl) to provide an intermediate of formula (XIV). Alternatively, the reaction of a compound of formula (VI) (when PG²=OCOt-Bu) with the lithium salt of an oxazolidinone, in a suitable solvent (e.g. THF), may also provide a compound of formula (XIII).

The compound of formula (XIV) will undergo an [3+2]-azomethine ylide cycloaddition by reaction with the compound of general formula (XI), to provide diastereomers (XV) and (XVI) which can be separated by chromatography or crystallisation and hydrolysed to give a pyrrolidine of formula (III).

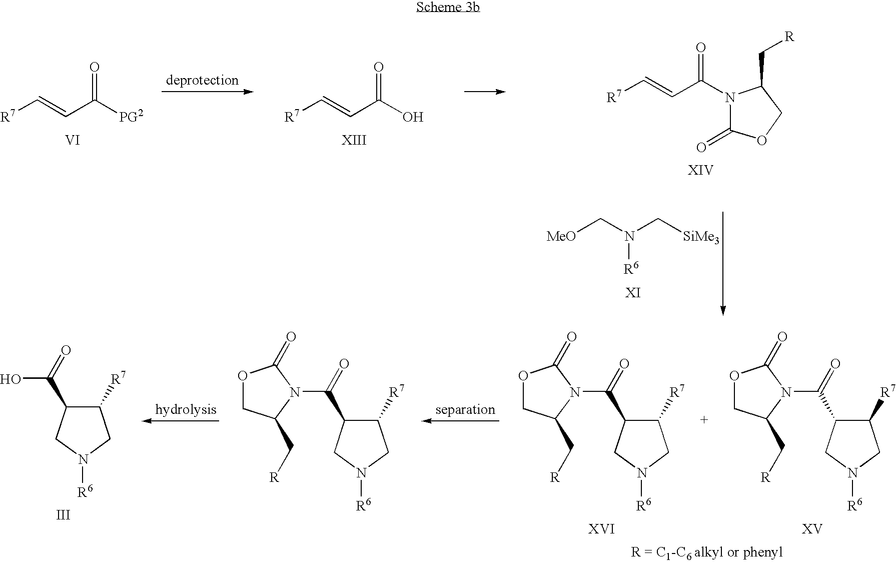

Scheme 4 illustrates that the synthesis of protected pyrrolidine acid intermediates of general formula (IV) can be achieved using a similar method to the process described hereinbefore for the intermediate of general formula (III) with the exception that the intermediate of general formula (XIIA) contains a nitrogen protecting group which may be removed subsequently in the synthetic scheme. Once the protecting group is removed, using any suitable conventional techniques, alternative R⁶ groups may be introduced by the methods described in scheme 2.

Pyrrolidines of general formula IV bearing a nitrogen protecting group may also be obtained enantioselectively by employment of an oxazolidinone chiral auxiliary, in a similar manner to that described in Scheme 3b.

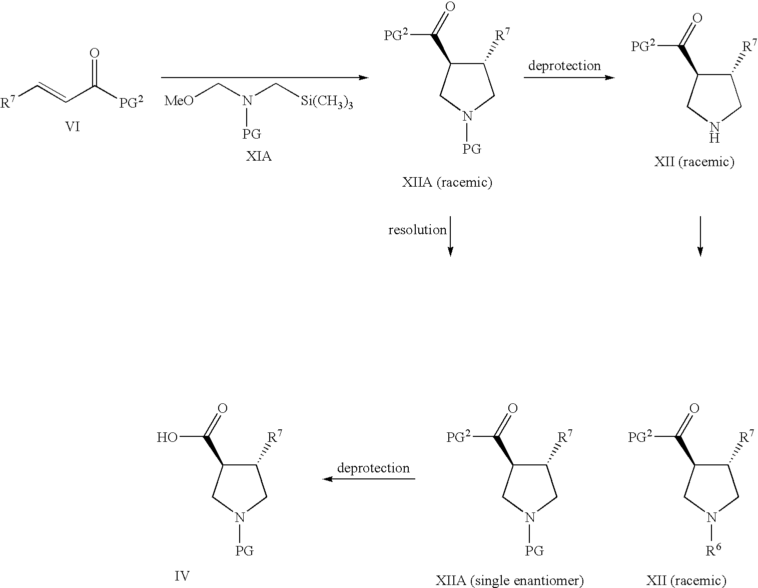

In respect of compounds (VI), (XIA), (XIIA), (XII) and (IV) in Scheme 4 the definitions of R⁶ and R⁷ are as defined hereinbefore for compounds of formula (Ia) unless stated otherwise. In formulae (XIA), (XIIA), and (IV), PG is selected from suitable nitrogen protecting groups. In formulae (VI), (XII) and (XIIA) PG² is selected from suitable carboxylic acid protecting groups.

Synthesis of azomethine ylid precursor compounds of general formula (XI) and (XIA) can be achieved as illustrated in scheme 5. Thus, a primary amine of general formula (XVII) may be alkylated by treatment with chloromethyltrimethylsilane, optionally neat or in an inert solvent, heating the reaction if required. The resulting intermediates (XVIII) can then be reacted with formaldehyde in methanol in the presence of a suitable base, such as potassium carbonate or tert-butylamine, to afford the intermediates (XI). To produce intermediates (XIA) containing a nitrogen protecting group a similar reaction sequence can be followed.

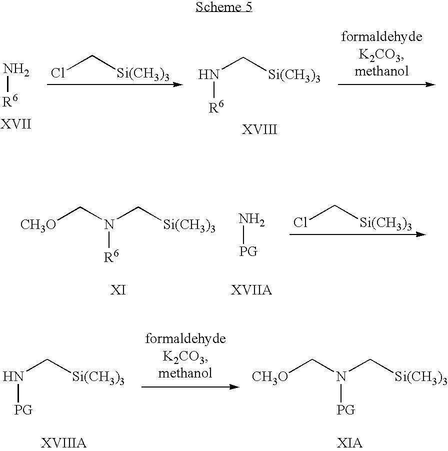

In respect of compounds (XVII), (XVIIA), (XVIII), (XVIIIA), (XI) and (XIA) in Scheme 5 the definitions of R⁶ are as defined hereinbefore for compounds of formula (Ia) unless stated otherwise. In formulae (XVIIA), (XVIIIA), (XIA), PG is selected from suitable nitrogen protecting groups.

The piperidines of general formula (II) may be formed as mixtures of diastereomers and separation of these diastereoisomers may be achieved at an appropriate stage by conventional techniques, e.g. by fractional crystallisation, chromatography or H.P.L.C. In addition, certain of these diastereomers may be racemic and require resolution into their constituent enantiomers, which can be achieved by standard resolution techniques, such as by H.P.L.C. using a suitable chiral support or by fractional crystallisation of the diastereoisomeric salts formed by reaction of the racemate with a suitable optically active acid. Alternatively, racemic piperidines of formula (II) may be coupled to optically active acids of formula (III) or (IV) to form mixtures of diastereomers which can be separated by standard techniques e.g. by fractional crystallisation, chromatography or H.P.L.C.

As illustrated in Scheme 6, piperidine intermediates of general formula (II), where R²═OH, can be prepared by addition of organometallic nucleophiles to ketones of general formula (XIX) containing a suitable nitrogen protecting group to furnish intermediates of general formula (XX). Such nucleophilic addition is generally carried out at low temperature in an anhydrous ethereal or non-polar solvent, using Grignard, organolithium or other suitable organometallic reagent. These organometallic reagents can be made by halogen-metal exchange using a suitable halide precursor, Y—Br or Y—I and n-butyl lithium or t-butyl lithium. Suitable protecting groups include Bn, which may be removed by hydrogenation or Boc, which may be removed by treatment with an acid such as TFA, or PMB which may be removed by treatment with DDQ, CAN or chloroethylchloroformate, to afford the desired piperidine intermediate of general formula (II). With certain protecting groups and under certain conditions the protecting group may be labile to treatment with the organometallic reagent, and so both transformations may be accomplished in one step. e.g. when PG=Boc the protecting group may sometimes be cleaved when intermediates of formula (XIX) are treated with an organometallic reagent.

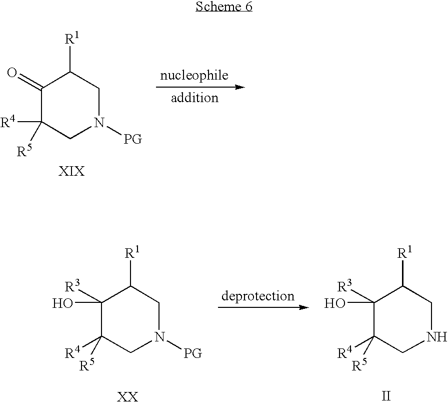

In respect of compounds (XIX), (XX), and (II) in scheme 6 the definitions of R¹, R³, R⁴ and R⁵ are as defined hereinbefore for compounds of formula (I) unless stated otherwise. In formulae (XIX), (XX), PG is selected from suitable nitrogen protecting groups. Compounds of formula (XIX) will be well-known to those skilled in the art with reference to literature precedents and/or the preparations herein.

In addition, Scheme 7 illustrates that under forcing reduction conditions, such as hydrogenation at high pressure and or temperature, or strong acid plus triethylsilane, intermediate compounds of formula general formula (II), where R²═OH may be converted into further intermediate compounds of general formula (II) where R²═H. In certain cases protection of the piperidine nitrogen atom may be required to facilitate this transformation. Thus, intermediates of general formula (XX) may be converted into further intermediate compounds of general formula (XXI) where R²═H, and then subsequently deprotected to provide compounds of general formula (II) where R²═H.

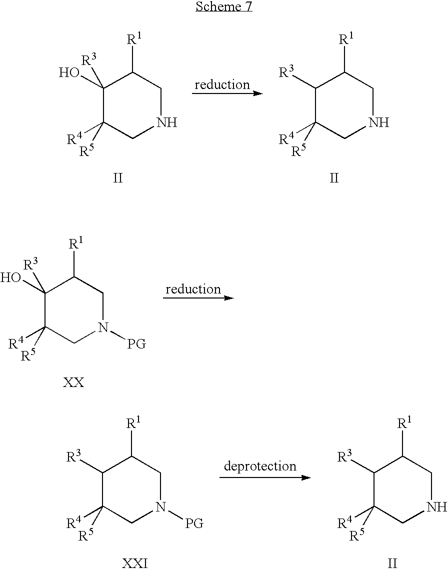

In respect of compounds (XX), (XXI) and (II) in Scheme 7 the definitions of R¹, R³, R⁴ and R⁵ are as defined hereinbefore for compounds of formula (Ia) unless stated otherwise. In formulae (XX) and (XXI), PG is selected from suitable nitrogen protecting groups.

As illustrated in Scheme 8, piperidine intermediates of general formula (II), where R²═NH₂, can be prepared by addition of organometallic nucleophiles to imines of general formula (XXII) containing suitable nitrogen protecting groups to furnish intermediates of general formula (XXIII). Such nucleophilic addition is generally carried out at low temperature in an anhydrous ethereal or non-polar solvent, using Grignard, organolithium or other suitable organometallic reagent. These organometallic reagents can be made by halogen-metal exchange using a suitable halide precursor, Y—Br or Y—I and n-butyl lithium or t-butyl lithium. Imines of formula (XXII) are available from ketones of formula (XIX) by reaction with the appropriate amine under suitable conditions, for example by carrying out the reaction in toluene at reflux with a Dean and Stark trap fitted to allow for azeotropic removal of water. Suitable protecting groups include Bn, which may be removed by hydrogenation, or Boc, which may be removed by treatment with an acid such as TFA, or PMB which may be removed by treatment with DDQ, CAN or chloroethylchloroformate, to afford the desired piperidine intermediate of general formula (II).

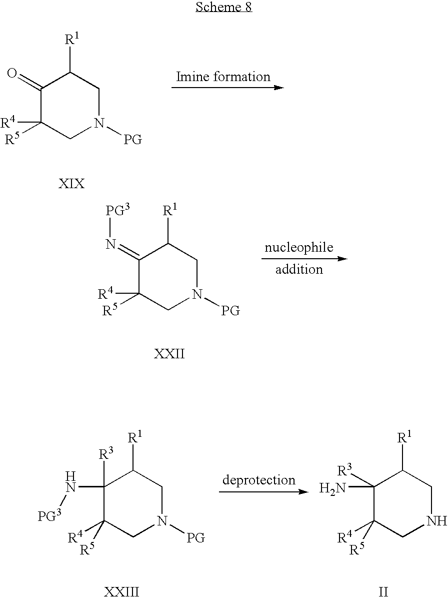

In respect of compounds (XIX), (XXII) and (XXIII) in Scheme 8 the definitions of R¹, R³, R⁴ and R⁵ are as defined hereinbefore for compounds of formula (Ia) unless stated otherwise. In formulae (XIX) (XXII) and (XXIII), PG and PG³ are selected from suitable nitrogen protecting groups.

As illustrated in Scheme 9, piperidine intermediates of general formula (II), where R¹═R²═OH, can be prepared by dihydroxylation of alkenes of general formula (XXIV) containing suitable nitrogen protecting groups to furnish intermediates of general formula (XXV). Many methods are available to carry out such a dihydroxylation reaction but particularly suitable is the asymmetric dihydroxylation reaction developed by Sharpless (Chemical Reviews 1994, 94, 2483) which generates a cis diol of known stereochemistry and usually in very high enantiomeric excess. Suitable protecting groups include Bn, which may be removed by hydrogenation, or Boc, which may be removed by treatment with an acid such as TFA, to afford the desired piperidine intermediate of general formula (II). Similarly, piperidine intermediates of general formula (II), where R⁴═R²═OH, can be prepared by dihydroxylation of alkenes of general formula (XXVI) to give intermediates of general formula (XXVII). Removal of the protecting group then gives the piperidine of formula (II).

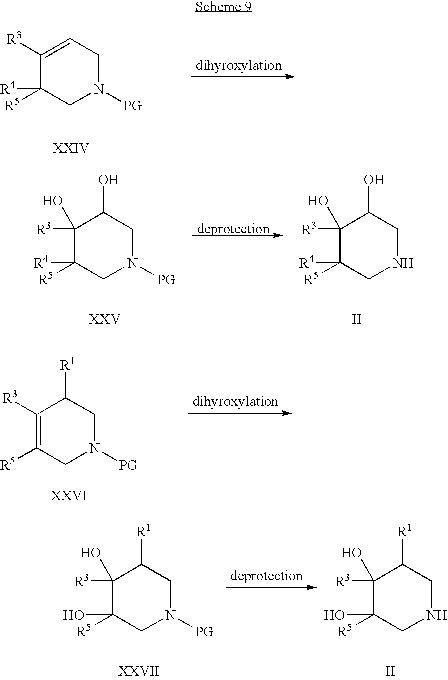

In respect of compounds (XXIV), (XXV), (XXVI) and (XXVII) in Scheme 9 the definitions of R¹, R², R³, R⁴ and R⁵ are as defined hereinbefore for compounds of formula (I) unless stated otherwise. In formulae (XXIV), (XXV), (XXVI) and (XXVII), PG is selected from suitable nitrogen protecting groups. Compounds of formulae (XXIV) and (XXVI) will be well-known to those skilled in the art with reference to literature precedents and/or the preparations herein.

In addition, scheme 10 illustrates that intermediate compounds of general formula (XXV) may be converted into further intermediate compounds of general formula (XXVIII) or (XXIX) which on deprotection give piperidines of general formula (II), where R¹═OC₁-C₄alkyl, R²═OH and R¹═R²═OC₁-C₄alkyl respectively. Conversion of intermediate compounds of formula (XXV) to compounds of formula (XXIX) may be achieved by the standard Williamson ether synthesis. That is, the alcohol groups in compounds of general formula (XXV) may be deprotonated with a strong base such as sodium hydride, in an anhydrous solvent, such as tetrahydrofuran or dimethylformamide, and the resulting anion reacted with an alkyl halide, heating the reaction if necessary. Alternatively, intermediates of formula (XXV) can be converted to compounds of general formula (XXVIII) by selectively alkylating only the less hindered secondary alcohol. Suitable conditions include reacting a diol of formula (XXV) with an excess of alkyl halide in a mixture of aqueous sodium hydroxide and toluene in the presence of a phase transfer catalyst such as tetrabutylammonium hydrogen sulfate.

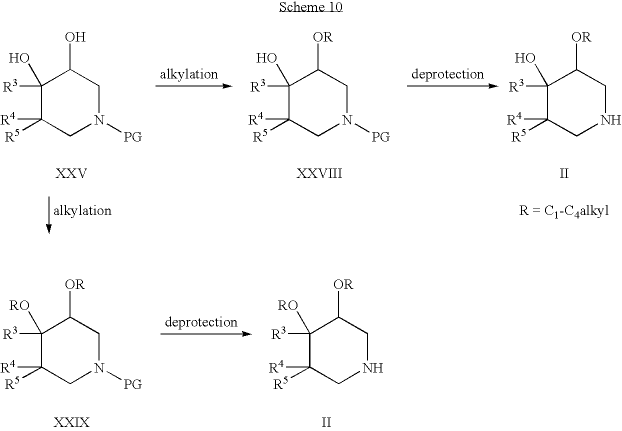

In respect of compounds (XXV), (XXVIII) and (XXIX) in Scheme 10 the definitions of R³, R⁴ and R⁵ are as defined hereinbefore for compounds of formula (Ia) unless stated otherwise. In formulae (XXV), (XXVIII) and (XXIX), PG is selected from suitable nitrogen protecting groups.

The skilled man will appreciate that, in addition to protecting nitrogen groups, as discussed hereinbefore, at various times during the synthesis of the compounds of formula Ia, it may be necessary to protect further groups, such as for example, hydroxy groups with a suitable protecting group, then remove the protecting group. Methods for deprotection of any particular group will depend on the protecting group. For examples of protection/ deprotection methodology see “Protective groups in Organic synthesis”, TW Greene and PGM Wutz. For example, where a hydroxy group is protected as a methyl ether, deprotection conditions comprise refluxing in 48% aqueous HBr, or by stirring with borane tribromide in dichloromethane. Alternatively where a hydroxy group is protected as a benzyl ether, deprotection conditions comprise hydrogenation with a palladium catalyst under a hydrogen atmosphere.

All of the above reactions and the preparations of novel starting materials used in the preceding methods are conventional and appropriate reaction conditions for their performance or preparation as well as procedures for isolating the desired products will be well-known to those skilled in the art with reference to literature precedents and the Examples and Preparations herein.

Experimental

The invention is illustrated by the following non-limiting examples in which the following abbreviations and definitions are used:
APCI atmospheric pressure chemical ionisation mass spectrum
[α]_{D} specific rotation at 589 nm.
Arbocel® filter agent
δ chemical shift
d Doublet
dd double doublet
EI electrospray ionisation
Ex Example
GC-MS gas chromatography mass spectrometry
HPLC high performance liquid chromatography
HRMS high resolution mass spectrum
LC-MS liquid chromatography mass spectrometry
LRMS low resolution mass spectrum
m Multiplet
m/z mass spectrum peak
NMR nuclear magnetic resonance
Prec Precursor
Prep Preparation
psi pounds per square inch
q Quartet
s Singlet
t Triplet
tlc thin layer chromatography

For synthetic convenience whilst in many instances compounds have been initially isolated in their free-base form, these have often been converted to their corresponding hydrochloride salts for analytical identification purposes. For the avoidance of doubt both the free-base and HCl salt forms are considered provided herein.

### EXAMPLES

### Example 1

### (3S,4R)-1-{[(3S,4R)-1-tert-butyl-4-(2,4-difluorophenyl)pyrrolidin-3-yl]carbonyl}-3-methyl-4-phenylpiperidin-4-ol hydrochloride

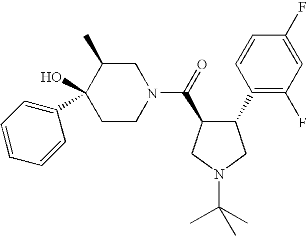

1-Propylphosphonic acid cyclic anhydride (50% in ethyl acetate, 0.37 mL, 0.62 mmol) was added to a mixture of (3S,4R)-3-methyl-4-phenylpiperidin-4-ol (synthesised according to J. Med. Chem. 1991, 34, 194) (100 mg, 0.52 mmol), triethylamine (0.22 mL, 1.56 mmol) and the acid of preparation 5 (200 mg, 0.62 mmol) in dichloromethane (5 mL) and the mixture was stirred at room temperature for 16 hours. Saturated aqueous sodium hydrogen carbonate solution (20 mL) was added to the reaction mixture and this was then extracted with dichloromethane (2×20 mL). The combined organic extracts were dried (MgSO₄), filtered and evaporated. The residue was purified by column chromatography (silica) eluting with dichloromethane/methanol (100% dichloromethane increasing polarity to 10% methanol in dichloromethane) to give the title compound as a white foam (203 mg, 86%). This was taken up in dichloromethane (3 mL) and converted to the hydrochloride salt by the addition of 2M ethereal HCl (2 mL). The solvent was removed in vacuo, the residue was taken up in dichloromethane and the salt was precipitated by the addition of pentane. The supernatant was removed and the solid was dried in vacuo to give the title compound (202 mg). ¹H NMR (CDCl₃, 400 MHz) δ 0.55-0.59 (m, 3H), 0.71 (dt 1H), 1.25-2.01 (m, 13H), 2.55-3.00 (m, 1H), 3.35-3.77 (m, 5 H) 4.00-4.55 (m, 3H) 6.79-6.88 (m, 1H), 6.93-7.00 (m, 1H), 7.10-7.11 (d, 1H), 7.22-7.37(m, 4H), 7.93-8.07 (m, 1H), 12.75 (br, s, 1H); LRMS (APCI⁺) 457 [MH⁺]; [α]_{D}²⁵=−80.8 (c=0.25, MeOH).

### Examples 2-17

The following compounds of formula Ii, i.e. compounds of general formula I where n=1 and R⁷=2,4-difluorophenyl, were prepared by the method described for Example 1 starting from the appropriate amine and acid precursors, as indicated. In some cases the desired product was isolated and characterised as the free base rather than the hydrochloride salt.

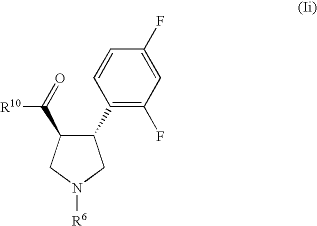

| | | | Prec. | Prec. | |
|---|---|---|---|---|---|
| Ex. | R⁶ | R¹⁰ | acid | amine | Data |
| 2 | 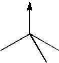 | 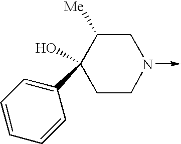 | Prep. 5 | Ref. a | 1H NMR (CDCl3, 400 MHz) δ0.38 and 0.63 (2 × d, 3H) 0.75-0.85 (m, 1H), 1.50-2.03 (m,12H), 2.64-3.12 (m, 2H), 3.43-3.78 (m, 5H), 4.08-4.54 (m, 3H),6.80-6.90 (m, 1H), 6.95-7.03 (m,1H) 7.12 (d, 1H), 7.22-7.38 (m,4H), 7.93-7.99 and 8.13-8.18(2 × m, 1H), 12.85 (br, s, 1H);LRMS (APCl+) 457 [MH+];[α]_{D}²⁵ = −32.6 (c = 0.24, MeOH) |
| 3 | 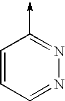 | 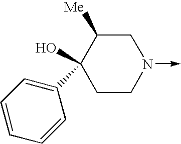 | Prep. 11 | Ref. b | 1H NMR (CD3OD, 400 MHz) δ0.57 and 0.62 (2 × d, 3H), 0.88-0.97 (m, 1H), 1.45-1.48 and1.64-1.71 and 1.92-2.09 (3 × m,3H), 2.77-2.83 and 2.97-3.06and 3.47-3.54 (3 × m, 2H), 3.74-4.50 (m, 8H), 7.03-7.24 (m, 4H),7.30-7.36 (m, 2H), 7.44-7.46 and7.50-7.56 and 7.61-7.67 (3 × m,2H), 7.76-7.82 (m, 1H), 7.88-7.92 (m, 1H), 8.56 (d, 1H);LRMS (APCl+) 479 [MH+];[α]_{D}²⁵ = −57.6 (c = 0.25, MeOH) |
| 4 | 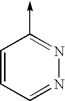 | 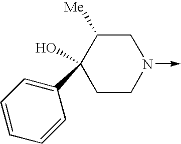 | Prep. 11 | Ref. a | ¹H NMR (CD₃OD, 400 MHz) δ0.45 and 0.62 (2 × d, 3H), 0.81-0.91 (m, 1H), 1.61-1.74 and2.00-2.06 (2 × m, 2H), 2.74-2.80and 3.02-3.28 (2 × m, 3H), 3.68-4.50 (m, 8H), 7.01-7.65 (m, 8H),7.70-7.73 (m, 1H), 7.84-7.88 (m,1H), 8.55 (d, 1H); LRMS (APCl⁺)479 [MH⁺]; [α]_{D}²⁵ = −13.9(c = 0.26, MeOH) |
| 5 | 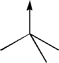 | 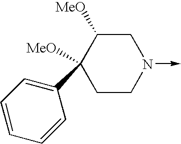 | Prep. 5 | Prep. 18 | ¹H NMR (CD₃OD, 400 MHz) δ1.49 (s, 9H), 1.84-1.92, 2.02-2.14 and 2.30-2.37 (3 × m, 3H),2.76-4.32 (m, 16H), 7.01-7.13(m, 2H), 7.27-7.44 (m, 5H), 7.60(m, 1H); LRMS (APCl⁺) 487[MH⁺]; [α]_{D}²⁵ = −29.4(c = 0.27, MeOH) |
| 6 | 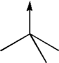 | 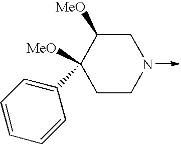 | Prep. 5 | Prep. 21 | ¹H NMR (CD₃OD, 400 MHz) δ1.48 (s, 9H), 1.85 (m, 1H), 2.08(m, 1H), 2.86-4.50 (m, 17H),7.05-7.44 (m, 7H), 7.65 (m, 1H);LRMS (APCl⁺) 487 [MH⁺];[α]_{D}²⁵ = −29.5 (c = 0.32, MeOH) |
| 7 | 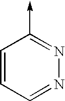 | 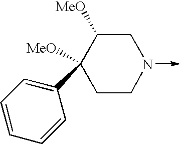 | Prep. 11 | Prep. 18 | ¹H NMR (CD₃OD, 400 MHz) δ1.73-1.83, 1.99-2.16 and 2.43-2.48 (3 × m, 3H), 2.94-3.43 (m,8H), 3.69-3.95 (m, 3H), 4.09-4.35 (m, 5H), 6.99-7.10 (m, 2H),7.27-7.59 (m, 6H), 7.78 (m, 1H),7.91 (dd, 1H), 8.56 (dd, 1H);LRMS (APCl⁺) 509 [MH⁺];[α]_{D}²⁵ = −21.1 (c = 0.27, MeOH) |
| 8 | 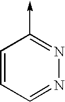 | 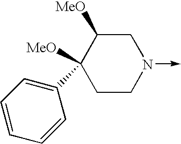 | Prep. 11 | Prep. 21 | ¹H NMR (CD₃OD, 400 MHz) δ1.94 (m,1H), 2.12 (m, 1H), 2.94-3.43 (m, 9H), 3.71-3.98 (m, 3H),4.09-4.29 (m, 4H), 4.47 (m, 1H),7.02-7.14 (m, 2H), 7.22-7.63 (m,6H), 7.76 (m, 1H), 7.90 (m, 1H),8.56 (m, 1H); LRMS (APCl⁺) 509[MH⁺]; [α]_{D}²⁵ = −18.2(c = 0.35, MeOH) |
| 9 | 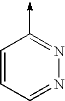 | 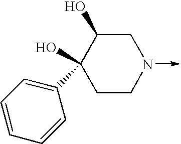 | Prep. 11 | Prep. 22 | ¹H NMR (CD₃OD, 400 MHz) δ0.88, 1.52, 1.76 and 1.93 (4 × m,2H), 2.88-3.17 and 3.40-3.58(2 × m, 3H), 3.76-3.98 (m, 3H),4.10-4.25 (m, 4H), 4.35 and 4.52(2 × m, 1H), 7.00-7.14 (m, 2H),7.20-7.25 (m, 3H), 7.30-7.36 (m,2H), 7.52 (m, 1H), 7.59 (m, 1H),7.75 (m, 1H), 7.88 (dd, 1H);LRMS (APCl⁺) 481 [MH⁺];[α]_{D}²⁵ = −64.3 (c = 0.38, MeOH) |
| 10 | 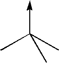 | 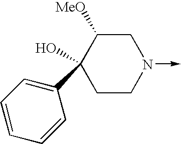 | Prep. 5 | Prep. 24 | ¹H NMR (CD₃OD, 400 MHz) δ1.51 (s, 9H), 1.59-1.68 (m, 1H),1.90-1.97 and 2.45-2.53 (2 × brm, 1H), 2.83-4.10 (m, 13H),4.35-4.38 and 4.59-4.62 (2 × m,1H), 7.04-7.25 and 7.31-7.35and 7.47-7,49 (3 × m, 7H), 7.56-7.60 and 7.68-7.74 (2 × m, 1H);LRMS (APCl⁺) 473 [MH⁺];[α]_{D}²⁵ = −25.0 (c = 0.22, MeOH) |
| 11 | 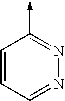 | 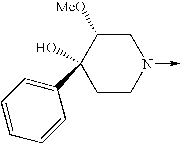 | Prep. 11 | Prep. 24 | ¹H NMR (CD₃OD, 400 MHz) δ1.54-1.75 and 1.94-2.01 and2.60-2.64 (3 × m, 3H), 2.89-3.06(m, 4H), 3.20-4.24 (m, 9H), 4.36-4.41 and 4.61-4.56 (2 × m, 1H),7.01-7.08 and 7.12-7.26 and7.31-7.37 and 7.49-7.52 and7.65-7.75 (4 × m, 8H), 7.72-7.75(m, 1H), 7.86-7.90 (m, 1H) 8.57(dd, 1H); LRMS (APCl⁺) 495[MH⁺]; [α]_{D}²⁵ = −8.1(c = 0.25, MeOH) |
| 12 | 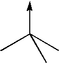 | 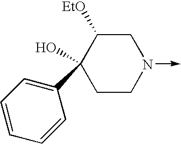 | Prep. 5 | Prep. 26 | ¹H NMR (CD₃OD, 400 MHz) δ0.77 and 0.87 (2 × t, 3H), 1.50 (s,9H), 1.60-1.71 and 1.95-2.02and 2.55-4.05 (3 × m, 13H), 4.37-4.40 and 4.54-4.57 (2 × m, 1H),7.03-7.34 (m, 6H), 7.48 (d, 1H),7.55-7.61 and 7.68-7.73 (2 × m,1H); LRMS (APCl⁺) 487 [MH⁺];[α]_{D}²⁵ = −27.1 (c = 0.28, MeOH) |
| 13 | 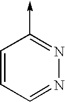 | 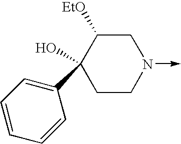 | Prep. 11 | Prep. 26 | ¹H NMR (CD₃OD, 400 MHz) δ0.82 and 0.88 (2 × t, 3H), 1.57-1.77 and 1.97-2.05 (2 × m, 2H),2.65-2.69 and 2.76-2.83 and2.90-3.14 (3 × m, 3H), 3.20-3.48and 3.59-3.63 and 3.78-4.23(3 × m, 9H), 4.38-4.43 and 4.55-4.65 (2 × m, 1H), 7.01-7.26 (m,4H), 7.31-7.36 (m, 2H), 7.47-7.52 and 7.61-7.67 (2 × m, 2H),7.74-7.78 (m, 1H), 7.89 (dd, 1H),8.56 (d, 1H); LRMS (APCl⁺) 509[MH⁺]; [α]_{D}²⁵ = −9.1(c = 0.31, MeOH) |
| 14 | 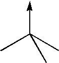 | 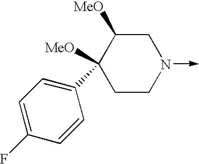 | Prep. 5 | Prep 29 | ¹H NMR (CD₃OD, 400 MHz) δ1.49 (s, 9H), 1.82-2.16 (m, 2H),2.80-3.14 (m, 8H), 3.45-4.08 (m,8H), 4.32 and 4.50 (2 × m, 1H),7.06-7.28 (m, 5H), 7.41-7.46 and7.56-7.68 (2 × m, 2H); LRMS(APCl⁺) 505 [MH⁺]; [α]_{D}²⁵ =−31.9 (c = 0.25, MeOH) |
| 15 | 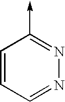 | 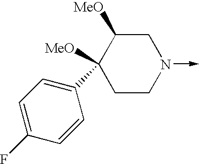 | Prep. 11 | Prep. 29 | ¹H NMR (CD₃OD, 400 MHz) δ1.00-1.11 and 1.91-2.18 (2 × m,2H), 2.94-3.42 (m, 9H), 3.71-4.32 and 4.48-4.54 (2 × m, 8H),7.02-7.14, 7.23-7.29 and 7.44-7.62 (3 × m, 7H), 7.76 (m, 1H),7.91 (m, 1H), 8.56 (m, 1H);LRMS (APCl⁺) 527 [MH⁺];[α]_{D}²⁵ = −17.6 (c = 0.23, MeOH) |
| 16 | 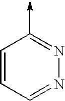 | 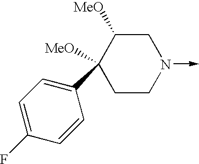 | Prep. 11 | Prep. 32 | ¹H NMR (CD₃OD, 400 MHz) δ1.76, 2.01-2.17, 2.39 (3 × m,2H), 2.91-3.45 (m, 9H), 3.66-400(m, 3H), 4.04-4.26 (m, 4H), 4.32(m, 1H), 6.97-7.16 (m, 4H), 7.32(m, 1H), 7.42-7.61 (m, 2H), 7.72-7.84 (m, 1H), 7.91 (m, 1H), 8.57(m, 1H); LRMS (APCl⁺) 527[MH⁺]; [α]_{D}²⁵ = −18.9(c = 0.20, MeOH) |
| 17 | 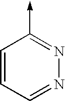 | 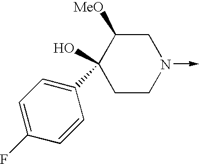 | Prep. 11 | Prep. 34 | ¹H NMR (CD₃OD, 400 MHz) δ1.04, 1.54, 1.70, 1.90 (4 × m, 2H),2.82-3.25 (m, 5H), 3.38-3.93 (m,4H), 3.96-4.24 (m, 4H),4.35, 4.70 (2 × m, 1H), 6.93-7.12(m, 5H), 7.23-7.33 (m, 1H), 7.37-7.62 (m, 3H), 8.43-8.50 (m, 1H);LRMS (ES⁺) 513 [MH⁺] |

Ref. a - (3R,4S)-3-methyl-4-phenylpiperidin-4-ol was synthesised according to J. Med. Chem. 1991, 34, 194
Ref. b - (3S,4R)-3-methyl-4-phenylpiperidin-4-ol was synthesised according to J. Med. Chem. 1991, 34, 194

### Example 18

### (3S,4S)-1-{[(3S,4R)-4-(2,4-Difluorophenyl)-1-propionylpyrrolidin-3-yl]carbonyl}-3,4-dimethoxy-4-phenylpiperidine

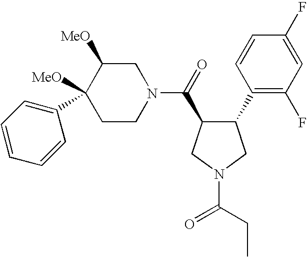

The hydrochloride salt of the amine of preparation 15 (100 mg, 0.21 mmol) was suspended in dichloromethane (2 mL) and triethylamine (90 μL, 0.64 mmol) was added to give a clear solution. Propionyl chloride (27 μl, 0.32 mmol) was then added and the reaction mixture was stirred at room temperature for 16 hours. The reaction was quenched by the addition of saturated aqueous sodium hydrogen carbonate solution (10 mL) and the mixture was extracted with ethyl acetate (10 mL). The organic layer was washed with brine, dried (MgSO₄) and evaporated. The residue was purified by column chromatography (silica) eluting with dichloromethane/methanol/ammonia (99:1:0.1 increasing polarity to 98:2:0.2) to give the title compound as a white foam (76 g, 74%). ¹H NMR (CDCl₃, 400 MHz) δ 1.00-1.50 (m, 4H), 1.83-2.19 (m, 1H), 2.26-2.38 (m, 2H), 2.82-3.19 (m, 8H), 3.26-4.20 (m, 8H), 4.40-4.61 (m, 1H), 6.81-6.96 (m, 2H), 7.19-7.42 (m, 6H); LRMS (APCI⁺) 487 [MH⁺]; [α]_{D}²⁵=−25.4 (c=0.18, MeOH).

### Example 19

### Methyl(3R,4S)-3-(2,4-difluorophenyl)-4-{[(3S,4S)-3,4-dimethoxy-4-phenylpiperidin-1-yl]carbonyl}pyrrolidine-1-carboxylate

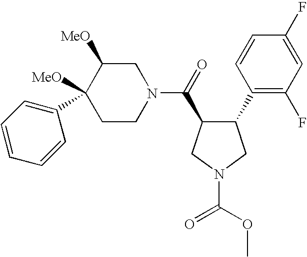

The title compound was prepared from the hydrochloride salt of the amine of preparation 15 according to the method of Example 18 using methyl chloroformate instead of propionyl chloride. ¹H NMR (CDCl₃, 400 MHz) δ 0.82-1.39 (m, 1H), 1.91-2.19 (m, 2H), 2.81-3.28 (m, 7H), 3.28-4.05 (m, 11H), 4.40-4.53 (m, 1H), 6.78-6.93 (m, 2H), 7.18-7.43 (m, 6H); LRMS (APCI⁺) 489 [MH⁺]; [α]_{D}²⁵=−18.6 (c=0.16, MeOH).

### Example 20

### Ethyl(3R,4S)-3-(2,4-difluorophenyl )-4-{[(3S,4S)-3,4-dimethoxy-4-phenylpiperidin-1-yl]carbonyl}pyrrolidine-1-carboxylate

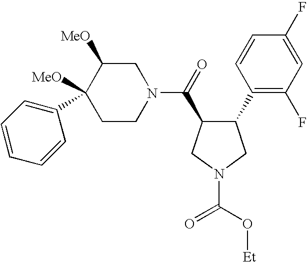

The title compound was prepared from the hydrochloride salt of the amine of preparation 15 according to the method of Example 18 using ethyl chloroformate instead of propionyl chloride. ¹H NMR (CDCl₃, 400 MHz) δ 1.20-1.36 (m, 3H), 1.92-2.19 (m, 2H), 2.82-2.96 (m, 1H), 2.98-3.18 (m, 7H), 3.27-4.22 (m, 10H), 4.41-4.62 (m, 1H), 6.75-6.93 (m, 2H), 7.19-7.42 (m, 6H); LRMS (APCI⁺) 503 [MH⁺]; [α]_{D}²⁵=−25.4 (c=0.2, MeOH).

### Example 21

### (3S,4S)-1-{[(3S,4R)-4-(2,4-Difluorophenyl)-1-(tetrahydro-2H-pyran-4-yl)pyrrolidin-3-yl]carbonyl}-3,4-dimethoxy-4-phenylpiperidine hydrochloride

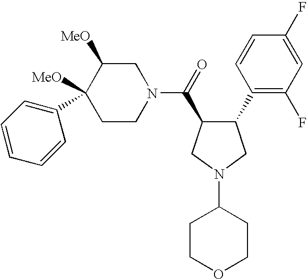

The hydrochloride salt of the amine of preparation 15 (100 mg, 0.21 mmol) was dissolved in ethanol (2 mL) with triethylamine (60 μL, 0.42 mmol) and stirred for 5 minutes. Tetrahydro-4H-pyran-4-one (30 μl, 0.32 mmol) was then added and the reaction mixture was stirred for a further 10 minutes before the addition of sodium triacetoxyborohydride (68 mg, 0.32 mmol). The reaction was stirred at room temperature for 16 hours and the solvent was then removed in vacuo. The residue was partitioned between water (15 mL) and ethyl acetate (20 mL) and the organic layer was washed with water (15 mL) and brine, dried (MgSO₄) and evaporated. The residue was purified by column chromatography (silica) eluting with dichloromethane/methanol/ammonia (99:1:0.1 increasing polarity to 97:3:0.3) to give the title compound as a colourless oil. This was dissolved in dichloromethane (2 mL) and 4M HCl in dioxane was added to form the hydrochloride salt. The solvent was removed in vacuo and the residue was azeotroped with toluene (10 mL) and then dichloromethane (2 mL) to give the title compound as a white foam (95 g, 82%). ¹H NMR (CDCl₃, 400 MHz) δ 1.52-1.70 (m, 2H), 1.73-2.16 (m, 4H), 2.35-2.43 (m, 1H), 2.63-3.70 (m, 17H), 3.92-4.03 (m, 3H), 4.44-4.68 (m, 1H), 6.62-6.90 (m, 2H), 7.22-7.53 (m, 6H); LRMS (APCI₊) 515 [MH⁺]; [α]_{D}²⁵=−23.6 (c=0.21, MeOH).

Preparations

Preparation 1

### 2-Methyl-N-[(trimethylsilyl)methyl]propan-2-amine

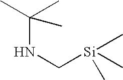

A procedure is given in J. Org. Chem. 53(1), 194, 1988 for the preparation of this intermediate. Alternative procedures are given below:

A solution of (chloromethyl)trimethylsilane (50 g, 408 mmol) and tert-butylamine (130 mL) under dry nitrogen was heated at 200° C. in a sealed tube for 18 hours before being quenched by the addition of 2M sodium hydroxide solution (700 mL). The resulting mixture was extracted with diethyl ether (3×100 mL) and the combined organic layers were distilled under dry nitrogen at 1 atmosphere to afford the title compound as a clear oil (62 g, 96%). ¹H NMR (CDCl₃, 400 MHz) δ 0.05 (s, 9H), 1.05 (s, 9H), 1.95 (s, 2H).

Alternative Preparation:

(Chloromethyl)trimethylsilane (100 mL, 730 mmol) and tert-butylamine (250 mL, 2400 mmol) were placed in a sealed bomb and heated with vigorous stirring for 18 hours. On cooling to room temperature, the slurry of the hydrochloride salts produced and residual excess tert-butylamine were poured into 4M sodium hydroxide solution (500 mL) and stirred vigorously for 1 hour. The aqueous layer was separated and the organic layer was stirred vigorously with water (3×500 mL) (the excess tert-butylamine is very water soluble, the product is only sparingly soluble). The residual organic layer was dried over sodium sulfate to give essentially pure 2-methyl-N-[(trimethylsilyl)methyl]propan-2-amine (105.4 g), which was used without further purification.

Preparation 2

### N-(Methoxymethyl)-2-methyl-N-[(trimethylsilyl)methyl]propan-2-amine

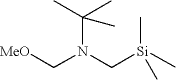

2-Methyl-N-[(trimethylsilyl)methyl]propan-2-amine (from preparation 1) (4.31 g, 27 mmol) was added to an ice-cooled mixture of methanol (1.29 mL, 31.8 mmol) and aqueous formaldehyde (37% w/v 2.49 mL, 33 mmol) over 45 minutes. The heterogeneous mixture was stirred at 0° C. for 2 hours and then solid potassium carbonate (325 mesh) (1.08 g, 13 mmol) was added and the mixture was stirred for 30 minutes at 0° C. The layers were separated and the aqueous phase was extracted with ethyl acetate (3×20 mL). The combined organic layers were dried over sodium sulfate, filtered, and evaporated under reduced pressure to give an 80:20 mixture of the title compound and unreacted tert-butyl[(trimethylsilyl)methyl]amine as a colourless oil (5.09 g). The mixture was used directly without further purification. ¹H NMR (CD₃OD, 400 MHz) δ 0.04 (s, 9H), 1.11 (s, 9H), 2.27 (s, 2H), 3.34 (s, 3H), 4.17 (s, 2H).

Preparation 3

### (4S)-4-Benzyl-3-[(2E)-3-(2,4-difluorophenyl)prop-2-enoyl]-1,3-oxazolidin-2-one

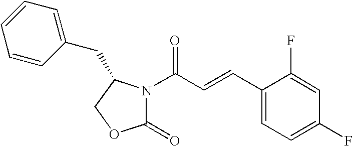

Oxalyl chloride (19 mL, 216 mmol) in dichloromethane (50 mL) was added dropwise to an ice-cooled stirred suspension of 2,4-difluorocinnamic acid (20.0 g, 108 mmol) in dichloromethane (400 mL) and N,N-dimethylformamide (0.4 mL) over 0.5 hours (waste gases from the reaction were scrubbed with a solution of concentrated sodium hydroxide). Once addition was complete, the reaction mixture was allowed to warm up to room temperature and was stirred at room temperature under nitrogen for 18 hours. The reaction mixture was then concentrated and azeotroped with dichloromethane (2×50 mL). The resulting acid chloride was dissolved in dichloromethane (50 mL) and this solution was added dropwise under nitrogen to a vigorously stirred suspension of lithium chloride (23.0 g, 540 mmol), triethylamine (76 mL, 540 mol) and (S)-(−)-4-benzyl-2-oxazolidinone (18.3 g, 103 mmol) in dichloromethane (400 mL) over 30 minutes. Once addition was complete, the reaction mixture was stirred at room temperature under nitrogen for 2.5 hours. The reaction mixture was diluted with dichloromethane (200 mL) and treated with a solution of 5% citric acid solution (500 mL). The organic layer was then separated and dried over magnesium sulfate. Filtration and evaporation of the dichloromethane gave the crude product as an orange oil. The crude material was dissolved in dichloromethane (100 mL) and the resulting solution was passed through a plug of silica, eluting with dichloromethane. The filtrate (1 L) was finally concentrated to afford 30.8 g of the product as a white solid. ¹H NMR (CDCl₃, 400 MHz) δ 2.85 (dd, 1H), 3.36 (dd, 1H), 4.22 (m, 2H), 4.80 (m, 1H), 6.90 (m, 2H), 7.68 (m, 5H), 7.68 (dd, 1H), 7.91 (d, 1H), 8.01 (dd, 1H); LRMS (APCI⁺) 344 [MH⁺].

Preparation 4a

### (4S)-4-Benzyl-3-{[(3R,4S)-1-tert-butyl-4-(2,4-difluorophenyl)pyrrolidin-3-yl]carbonyl}-1,3-oxazolidin-2-one

and

Preparation 4b

### (4S)-4-Benzyl-3-{[(3S,4R)-1-tert-butyl-4-(2,4-difluorophenyl)pyrrolidin-3-yl]carbonyl}-1,3-oxazolidin-2-one

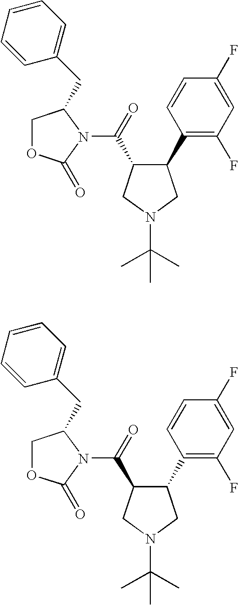

A stirred solution of (S)-4-benzyl-3-[3-(2,4-difluoro-phenyl)-acryloyl]-oxazolidin-2-one (from preparation 3) (1.70 g, 4.95 mmol) and N-(methoxymethyl)-2-methyl-N-[(trimethylsilyl)methyl]propan-2-amine (from preparation 2) (1.60 g, 5.94 mmol) in dichloromethane (15 mL) was treated with trifluoroacetic acid (0.075 mL, 1 mmol). The resulting mixture was stirred at room temperature under nitrogen for 4.5 hours. The reaction mixture was diluted with dichloromethane (50 mL) and treated with saturated aqueous sodium hydrogen carbonate solution (50 mL). The organic layer was separated and the aqueous layer was extracted with dichloromethane (50 mL). The organic fractions were combined and dried over magnesium sulfate. Filtration and evaporation of the dichloromethane gave the crude mixture of diastereoisomers.

Separation by column chromatography on silica gel with pentane:ethyl acetate 80/20 to 10/90 v/v, gradient elution, afforded firstly 0.74 g (1.67 mmol) of (4S)-4-benzyl-3-{[(3R,4S)-1-tert-butyl-4-(2,4-difluorophenyl)pyrrolidin-3-yl]carbonyl}-1,3-oxazolidin-2-one as a colourless oil, and then 0.82 g (1.85 mmol) of (4S)-4-benzyl-3-{[(3S,4R)-1-tert-butyl-4-(2,4-difluorophenyl)pyrrolidin-3-yl]carbonyl}-1,3-oxazolidin-2-one as a white solid.

(4S)-4-benzyl-3-{[(3R,4S)-1-tert-butyl-4-(2,4-difluorophenyl)pyrrolidin-3-yl]carbonyl}-1,3-oxazolidin-2-one—¹H NMR (CDCl₃, 400 MHz) δ 1.12 (s, 9H), 2.77 (dd, 1H), 2.85 (m, 1H), 3.25 (dd, 1H), 3.17-3.47 (m, 1H), 4.15 (m, 3H), 4.65 (m, 1H), 6.74 (t, 1H), 6.82 (t, 1H), 7.17-7.42 (m, 6H); LRMS (APCI⁺) 443 [MH⁺].

(4S)-4-benzyl-3-{[(3S,4R)-1-tert-butyl-4-(2,4-difluorophenyl)pyrrolidin-3-yl]carbonyl}-1,3-oxazolidin-2-one—¹H NMR (CDCl₃, 400 MHz) δ 1.12 (s, 9H), 2.72 (dd, 1H), 2.83 (m, 2H), 3.20 (m, 2H), 3.36 (t, 1H), 4.14 (m, 3H), 4.29 (m, 1H), 4.67 (m, 1H), 6.77 (t, 1H), 6.85 (t, 1H), 7.08 (m, 2H), 7.24 (m, 3H), 7.43 (m, 1H); LRMS (APCI⁺) 443 [MH⁺].

The full relative and absolute stereochemistry of (4S)-4-benzyl-3-{[(3S,4R)-1-tert-butyl-4-(2,4-difluorophenyl)pyrrolidin-3-yl]carbonyl}-1,3-oxazolidin-2-one was determined by X-ray analysis of crystals obtained from ethyl acetate/pentane.

Preparation 5

### (3S,4R)-1-tert-Butyl-4-(2,4-difluorophenyl)pyrrolidine-3-carboxylic acid hydrochloride

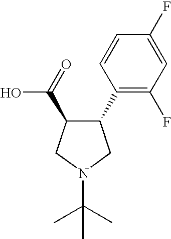

A solution of lithium hydroxide (0.93 g, 39 mmol) in water (15 mL) was added dropwise to a stirred suspension of (4S)-4-benzyl-3-{[(3S,4R)-1-tert-butyl-4-(2,4-difluorophenyl)pyrrolidin-3-yl]carbonyl}-1,3-oxazolidin-2-one (from preparation 4b) (8.63 g, 19.5 mmol) in tetrahydrofuran (50 mL). The resulting reaction mixture was then stirred at room temperature for 1.5 hours, diluted with water (50 mL) and extracted with ethyl acetate (4×150 mL). The aqueous layer was separated, treated with 2M aqueous hydrogen chloride solution (19.5 mL), concentrated to dryness and azeotroped with toluene (5×50 mL). The residual white solid was triturated with dichloromethane (40 mL) and insoluble lithium chloride was removed by filtration. The filtrate was then evaporated to afford the product as a white foam (5.05 g, 92%). ¹H NMR (CD₃OD, 400 MHz) δ 1.44 (s, 9H), 3.36 (m, 2H), 3.64 (t, 1H), 3.25 (dd, 1H), 3.88 (m, 3H), 6.98 (t, 2H), 7.55 (q, 1H); LRMS (APCI⁺) 284 [MH⁺].

Preparation 6

### (4S)-4-Benzyl-3-{[(3S,4R)-1-benzyl-4-(2,4-difluorophenyl)pyrrolidin-3-yl]carbonyl}-1,3-oxazolidin-2-one

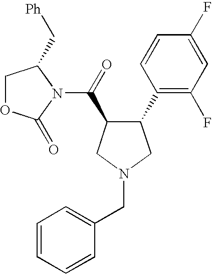

To a stirred solution of (4S)-4-benzyl-3-[(2E)-3-(2,4-difluorophenyl)prop-2-enoyl]-1,3-oxazolidin-2-one (from preparation 3) (46.83 g, 140 mmol) in dichloromethane (300 mL) was added N-methoxymethyl-N-(trimethylsilylmethyl)benzylamine (50.2 mL, 210 mmol) at room temperature. The solution was cooled to −12° C. and a solution of trifluoroacetic acid (1.05 mL) in dichloromethane (10 mL) was added dropwise. The reaction mixture was warmed to room temperature, stirred for 24 hours and saturated sodium hydrogen carbonate solution (180 mL) was added. The phases were separated and the aqueous phase was extracted with dichloromethane (180 mL). The organic extracts were combined, dried over magnesium sulfate, filtered and concentrated in vacuo. Purification of the residue by column chromatography using toluene:methyl tert-butyl ether (12:1) followed by dichloromethane:methyl tert-butyl ether (19:1) as the eluent afforded the title compound (which is the second eluting diastereomer), (63.0 g, 49%). ¹H NMR (CDCl₃, 400 MHz) δ 2.75 (m, 3H), 3.12 (t, 1H), 3.24 (m, 2H), 3.70 (q, 2H) 4.13 (m, 2H), 4.27 (q, 1H), 4.33 (m, 1H), 4.67 (m, 1H), 6.57 (m, 1H), 6.84 (t, 1H), 7.13 (m, 2H), 7.16 (m, 1H), 7.24-7.41 (m, 8H).

Preparation 7

### Methyl(3S,4R)-1-benzyl-4-(2,4-difluorophenyl)pyrrolidine-3-carboxylate

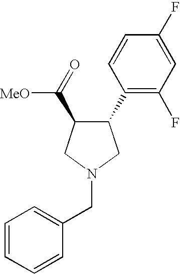

Samarium triflate (6.32 g, 10 mmol) was added to a stirred solution of (4R)-4-benzyl-3-{[(3S,4R)-1-benzyl-4-(2,4-difluorophenyl)pyrrolidin-3-yl]carbonyl}-1,3-oxazolidin-2-one (from preparation 6) (63 g, 130 mmol) in methanol (350 mL) at room temperature. The reaction mixture was stirred for 24 hours and the solvent was removed in vacuo. Dichloromethane (290 mL) was added followed by saturated sodium hydrogen carbonate solution (140 mL) and the mixture was stirred for 15 minutes. The resulting precipitate was filtered and washed with dichloromethane (250 mL) and water (25 mL). The phases were separated and the aqueous layer was extracted with dichloromethane (2×40 mL). The organic extracts were combined, dried over magnesium sulfate, filtered and concentrated in vacuo. The residue was suspended in warm cyclohexane (300 mL) and shaken till formation of a solid occurred. The mixture was allowed to stand at room temperature for 24 hours and the solid was filtered and washed with cold cyclohexane (150 mL). The filtrate was concentrated in vacuo to afford the desired compound, (38 g, 87%). ¹H NMR (CDCl₃, 400 MHz,) δ 2.67 (t, 1H), 2.86 (m, 1H), 2.93 (t, 1H), 3.04 (m, 2H), 3.64 (s, 3H), 3.65 (t, 1H), 3.84 (m, 1H), 6.72 (m, 1H), 6.80 (t, 1H), 7.23 (m, 2H), 7.29-7.38 (m, 5H); [α]²⁵_{D}=38 (c=0.5, MeOH).

Preparation 8

### Methyl(3S,4R)-4-(2,4-difluorophenyl)pyrrolidine-3-carboxylate

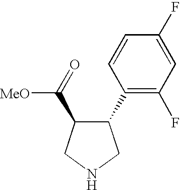

Palladium hydroxide (20% on carbon, 1 g) was added to a solution of methyl(3S,4R)-1-benzyl-4-(2,4-difluorophenyl)pyrrolidine-3-carboxylate (from preparation 7) (10 g, 30 mmol) in ethanol (50 mL) at room temperature. The reaction mixture was hydrogenated at 345 kPa pressure (50 psi) for 24 hours and then filtered through Arbocel®, washing with ethanol (50 mL). The solvent was removed in vacuo to give the desired compound as a colourless oil, (7.19 g, 98%). ¹H NMR (CD₃OD, 400 MHz) δ 2.60 (s, 1H), 2.91 (t, 1H), 3.08 (q, 1H), 3.31-3.44 (m, 1H), 3.50 (t, 1H), 3.63 (m, 1H), 3.66 (s, 3H), 6.76 (m, 1H), 6.84 (m, 1H), 7.20 (m, 1H); LRMS (EI⁺) 242 [MH⁺].

Preparation 9

### Methyl(3S,4R)-1-(6-chloropyridazin-3-yl )-4-(2,4-difluorophenyl)pyrrolidine-3-carboxylate

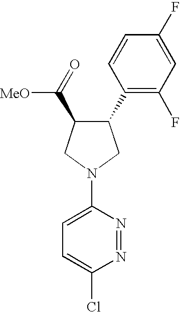

A mixture of methyl(3S,4R)-4-(2,4-difluorophenyl)pyrrolidine-3-carboxylate (from preparation 8) (10.4 g, 43.1 mmol) diisopropylethylamine (75 mL, 430 mmol) and 3,6-dichloropyridazine (22.5 g, 151 mmol) in tetrahydrofuran (90 mL) was heated at reflux for 16 hours. Analysis by tic indicated unreacted amine remaining so a further portion of 3,6-dichloropyridazine (12.0 g, 80.5 mmol) was added and heating was continued for a further 48 hours. After cooling to room temperature the solvent was removed in vacuo and the residue was partitioned between ethyl acetate (400 mL) and water (300 mL). The organic phase was washed with brine (200 mL), dried over magnesium sulfate and concentrated in vacuo. The residue was purified by column chromatography (silica) eluting with ethyl acetate/pentane (1:9 increasing polarity to 4:6) to give the title compound as a yellow oil (11.97 g, 78%). ¹H NMR (CDCl₃, 400 MHz) δ 3.45 (q, 1H), 3.64 (m, 1H), 3.69 (s, 3H), 3.85 (dd, 1H), 3.99-4.10 (m, 3H), 6.66 (d, 1H), 6.81-6.89 (m, 2H), 7.20-7.27 (m, 2H); LRMS (APCI⁺) 354 [MH⁺].

Preparation 10

### Lithium(3S,4R)-1-(6-chloropyridazin-3-yl)-4-(2,4-difluorophenyl)pyrrolidine-3-carboxylate

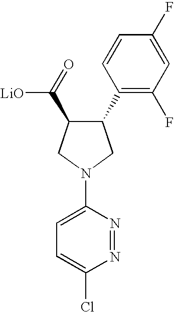

A solution of lithium hydroxide (1.58 g, 65.8 mmol) in water (45 mL) was added dropwise to a solution of methyl(3S,4R)-1-(6-chloropyridazin-3-yl)-4-(2,4-difluorophenyl)pyrrolidine-3-carboxylate (from preparation 9) (21.22 g, 60.0 mmol) in tetrahydrofuran (210 mL) and the mixture was stirred at room temperature for 16 hours. The solvent was removed in vacuo and the residue was azeotroped with toluene (3×80 mL) to give a white solid. This was dissolved in boiling methanol (200 mL) and the solution was allowed to cool to room temperature. Diethyl ether (˜150 mL) was then added gradually to give a white precipitate which was collected by filtration and washed with diethyl ether. Drying in vacuo gave the title compound (11.91 g, 57%). ¹H NMR (CD₃OD, 400 MHz) δ 3.34 (m, 1H), 3.46 (m, 1H), 3.71 (dd, 1H), 3.93-4.10 (m, 3H), 6.88-6.94 (m, 2H), 7.01 (d, 1H), 7.39 (d, 1H), 7.45 (m, 1H); LRMS (APCI⁻) 338 [M-H⁺].

Concentration of the filtrate in vacuo gave a yellow solid which was triturated with boiling ethanol (250 mL). After cooling the ethanol to room temperature diethyl ether (300 mL) was added to precipitate further solid which was collected by filtration and combined with the trituration residue. Drying in vacuo gave 6.81 g (33%) of the title compound.

Preparation 11

### (3S,4R)-4-(2,4-difluorophenyl)-1-pyridazin-3-ylpyrrolidine-3-carboxylic acid hydrochloride

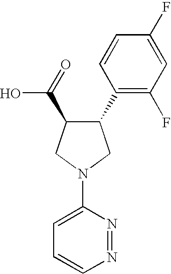

Lithium(3S,4R)-1-(6-chloropyridazin-3-yl)-4-(2,4-difluorophenyl)pyrrolidine-3-carboxylate (from preparation 10) (11.9 g, 34.4 mmol) was suspended in ethanol (110 mL) and 10% palladium on carbon (1.7 g) and 1-methyl-1,4-cyclohexadiene (25 mL, 222 mmol) were added. The mixture was heated at reflux for 2 hours and then a further portion of 1-methyl-1,4-cyclohexadiene (6 mL, 53 mmol) was added. After heating at reflux for a further 2 hours the mixture was cooled and filtered through Arbocel®, washing with ethanol. The filtrate was concentrated in vacuo and azeotroped with toluene (2×50 mL). The residue was triturated with dichloromethane (100 mL) then filtered and dried in vacuo. The yellow solid was taken up in acetone (175 mL) and water (175 mL) with slight heating and then treated with 2M ethereal HCl (50 mL) before being concentrated in vacuo. The residue was taken up in boiling isopropyl alcohol (650 mL), the mixture was filtered, diluted with diisopropyl ether (200 mL) and allowed to cool slowly to room temperature. The resulting precipitate was collected by filtration and washed with diethyl ether. The resulting white solid was boiled in toluene (80 mL) for 15 minutes, the suspension was allowed to cool to room temperature and then concentrated in vacuo. This was then repeated three times to give the title compound as a white solid (6.53 g, 62%). ¹H NMR (CD₃OD, 400 MHz) δ 3.61-3.77 (m, 2H), 3.96 (dd, 1H), 4.08-4.22 (m, 3H), 6.98-7.04 (m, 2H), 7.52 (m, 1H), 7.74 (dd, 1H), 7.89 (dd, 1H), 8.55 (dd, 1H); LRMS (APCI⁺) 306 [MH⁺].

Preparation 12

### 1-tert-Butyl 3-methyl(3S,4R)-4-(2,4-difluorophenyl)pyrrolidine-1,3-dicarboxylate

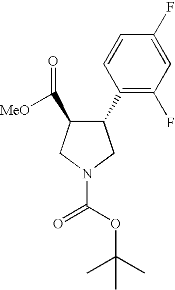

To a solution of methyl(3S,4R)-1-benzyl-4-(2,4-difluorophenyl)pyrrolidine-3-carboxylate, (from preparation 7) (1.0 g, 3.01 mmol), 1-methylcyclohexa-1,4-diene (1.25 mL, 11.12 mmol) and di-tert-butyl dicarbonate (0.72 g, 3.31 mmol) in ethanol (10 mL) was added palladium hydroxide on carbon (0.1 g) at room temperature. The resulting mixture was heated under reflux for 4 hours, cooled to room temperature and filtered through Arbocel®. The filtrate was concentrated in vacuo to give a residue which was partitioned between ethyl acetate (80 mL) and 10% citric acid solution (5 mL). The phases were separated and the organic layer was washed with brine (60 mL), dried over magnesium sulfate, filtered and concentrated in vacuo to give the desired product as a colourless oil (940 mg, 92%). ¹H NMR (CDCl₃, 400 MHz) δ 1.40 (s, 9H), 3.14-3.25 (m, 1H), 3.25-3.40 (m, 1H), 3.48-3.59 (m, 4H), 3.68-3.89 (m, 3H), 6.71-6.82 (m, 2H), 7.15 (m, 1H); LRMS (APCI) 242 [MH⁺-BOC]

Preparation 13

### (3S,4R)-1-(tert-Butoxycarbonyl)-4-(2,4-difluorophenyl)pyrrolidine-3-carboxylic acid

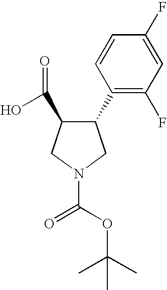

Lithium hydroxide (130 mg, 23.5 mmol) was added dropwise to a stirred solution of 1-tert-butyl 3-methyl (3S,4R)-4-(2,4-difluorophenyl)pyrrolidine-1,3-dicarboxylate (from preparation 12) (930 mg, 2.72 mmol) in tetrahydrofuran (10 mL) at room temperature. The reaction mixture was stirred for 48 hours, concentrated in vacuo and diluted with water (15 mL). The phases were separated and the aqueous phase was extracted with ethyl acetate (25 mL). The aqueous layer was acidified with 2M hydrochloric acid solution (2.7 mL) and further extracted with ethyl acetate (2×40 mL). The combined organic extracts were dried over magnesium sulfate, filtered, concentrated in vacuo and azeotroped with dichloromethane to give the desired product (775 mg, 87%). ¹H NMR (CDCl₃, 400 MHz) δ 1.45 (s, 9H), 3.23-3.46 (m, 2H), 3.56-3.65 (m, 1H), 3.74-3.93 (m, 3H), 6.75-6.87 (m, 2H), 7.20 (m, 1H); LRMS (APCI) 228 [MH⁺-BOC]; LRMS (APCI−)=326 [M−1].

Preparation 14

### tert-Butyl(3R,4S)-3-(2,4-difluorophenyl)-4-{[(3S,4S)-3,4-dimethoxy-4-phenylpiperidin-1-yl]carbonyl}pyrrolidine-1-carboxylate

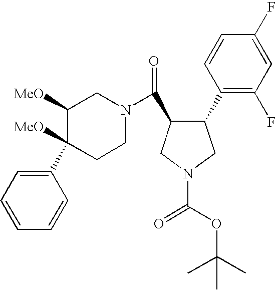

1-Propylphosphonic acid cyclic anhydride (50% in ethyl acetate, 1.6 mL, 2.66 mmol) was added to a mixture of (3S,4S)-3,4-dimethoxy-4-phenylpiperidine (from preparation 21) (589 mg, 2.66 mmol), triethylamine (0.74 mL, 5.32 mmol) and (3S,4R)-1-(tert-butoxycarbonyl)-4-(2,4-difluorophenyl)pyrrolidine-3-carboxylic acid (from preparation 13) (870 mg, 2.66 mmol) in dichloromethane (5 mL) and the mixture was stirred at room temperature for 16 hours. The reaction mixture was diluted with dichloromethane (20 mL) and washed with 10% aqueous potassium carbonate (20 mL) and brine (20 mL), then dried (MgSO₄) and evaporated. The residue was purified by column chromatography (silica) eluting with dichloromethane/methanol/ammonia (99:1:0.1 increasing polarity to 98:2:0.2) to give the title compound as a colourless oil (1.14 g, 81%). ¹H NMR (CDCl₃, 400 MHz) δ 1.42-1.50 (m, 9H), 1.91-2.16 (m, 2H), 2.84-3.18 (m, 7H), 3.29-4.10 (m, 9H), 4.40-4.62 (m, 1H), 6.78-6.91 (m, 2H), 7.21-7.42 (m, 6H); LRMS (APCI⁺) 531 [MH⁺].

Preparation 15

### (3S,4S)-1-{[(3S,4R)-4-(2,4-Difluorophenyl)pyrrolidin-3-yl]carbonyl}-3,4-dimethoxy-4-phenylpiperidine hydrochloride

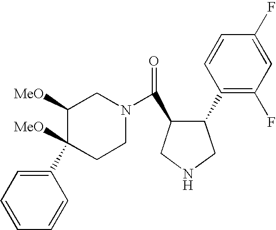

4M HCl in dioxane (10.75 mL) was added to a solution of tert-butyl (3R,4S)-3-(2,4-difluorophenyl)-4-{[(3S,4S)-3,4-dimethoxy-4-phenylpiperidin-1-yl]carbonyl}pyrrolidine-1-carboxylate (from preparation 14) (1.14 g, 2.15 mmol) in dichloromethane (11 mL) and the mixture was stirred at room temperature for 16 hours. The solvent was removed in vacuo and the residue was azeotroped with dichloromethane (30 mL) to give the title compound (859 mg, 86%) which was used without further purification. ¹H NMR (CD₃OD, 400 MHz) δ 1.01-2.42 (m, 2H), 3.00-3.16 (m, 7H), 3.27-3.32 (m, 2H), 3.48-3.98 (m, 7H), 4.22-4.50 (dd, 1H), 7.05-7.18 (m, 2H), 7.22-7.43 (m, 5H), 7.50-7.61 (m, 1H); LRMS (APCI⁺) 431 [MH⁺].

Preparation 16

### tert-Butyl(3R,4R)-3,4-dihydroxy-4-phenylpiperidine-1-carboxylate

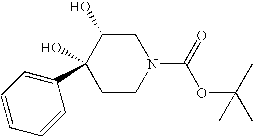

AD-mix β (21.58 g) and methanesulfonamide (1.47 g, 15.4 mmol) were added to water (80 mL) and tert-butanol (80 mL) and the mixture was stirred for 5 minutes at room temperature before being cooled to 0° C. tert-Butyl 4-phenyl-3,6-dihydropyridine-1(2H)-carboxylate (prepared according to Org. Lett. 2001, 3, 2317-2320) (4.0 g, 15.4 mmol) was then added in one portion and the reaction was stirred at 0° C. for 18 hours. Sodium sulfite (13.2 g, 105 mmol) was added and the mixture was stirred at room temperature for 30 minutes before being extracted with ethyl acetate (3×60 mL). The combined organic extracts were washed with 1M NaOH (40 mL), dried (MgSO₄) and evaporated. The residue was purified by column chromatography (silica) eluting with pentane/ethyl acetate (100% pentane increasing polarity to 50% EtOAc in pentane) to give the title compound as an off-white solid (4.18 g, 92%). ¹H NMR (CD₃OD, 400 MHz) δ 1.49 (s, 9H), 1.70 (dt, 1H), 1.90 (td, 1H), 3.00-3.20 (br m, 2H), 3.86-3.91 (m, 2H), 4.02-4.06 (m, 1H), 7.21 (tt, 1H), 7.33 (t, 2H), 7.50 (dd, 2H); LRMS (APCI⁺) 294 [MH⁺]; [α]_{D}²⁵=+19.8 (c=0.31, MeOH).

Preparation 17

### tert-Butyl(3R,4R)-3,4-dimethoxy-4-phenylpiperidine-1-carboxylate

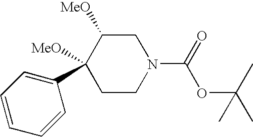

Sodium hydride (87 mg, 2.18 mmol) was added to a solution of tert-butyl(3R,4R)-3,4-dihydroxy-4-phenylpiperidine-1-carboxylate (from preparation 16) (200 mg, 0.68 mmol) in tetrahydrofuran (2 mL) and the mixture was stirred at room temperature for 1 hour. Methyl iodide (144 μL, 2.3 mmol) was then added dropwise over 5 minutes and the mixture was stirred for a further 4 hours. The reaction was cooled to 0° C. and quenched by the addition of water (20 mL). The reaction mixture was extracted with ethyl acetate (2×20 mL) and the combined extracts were washed with brine, dried (MgSO₄) and evaporated to give the title compound as a colourless oil (236 mg) which was used without further purification. ¹H NMR (CDCl₃, 400 MHz) δ 1.49 (s, 9H), 1.98-2.12 (m, 2H), 3.11 (s, 3H), 3.16 (s, 3H), 3.12-3.22 (m, 2H), 3.94 (br, 1H), 4.13 (br, 2H), 7.28-7.32 (m, 1H), 7.35-7.39 (m, 2H), 7.42-7.45 (m, 2H); LRMS (APCI⁺) 322 [MH⁺].

Preparation 18

### (3R,4R)-3,4-Dimethoxy-4-phenylpiperidine hydrochloride

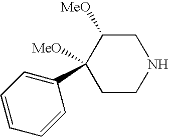

4M HCl in dioxane (4.4 mL) was added to a solution of tert-butyl(3R,4R)-3,4-dimethoxy-4-phenylpiperidine-1-carboxylate (from preparation 17) (230 mg) in dichloromethane (4 mL) and the mixture was stirred at room temperature for 16 hours. The solvent was removed in vacuo and the residue was azeotroped with diethyl ether (3×20 mL) to give the title compound as a white foam (207 mg) which was used without further purification. ¹H NMR (CD₃OD, 400 MHz) δ 2.37 (m, 2H), 3.11 (s, 3H), 3.19 (s, 3H), 3.23 (dd, 1H), 3.25 (dd, 1H), 3.29 (m, 2H), 3.66 (dd, 1H), 7.34-7.38 (m, 1H), 7.41-7.50 (m, 4H); LRMS (APCI⁺) 222 [MH⁺].

Preparation 19

### tert-Butyl(3S,4S)-3,4-dihydroxy-4-phenylpiperidine-1-carboxylate

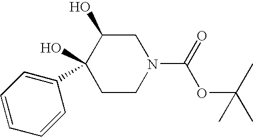

According to the method of preparation 16, but using AD-mix α instead of AD-mix β, tert-butyl 4-phenyl-3,6-dihydropyridine-1(2H)-carboxylate was converted to the title compound. ¹H NMR (CD₃OD, 400 MHz) δ 1.49 (s, 9H), 1.70 (dt, 1H), 1.90 (td, 1H), 3.00-3.20 (br m, 2H), 3.86-3.91 (m, 2H), 4.02-4.06 (m, 1 H), 7.21 (tt, 1H), 7.33 (t, 2H), 7.50 (dd, 2H); LRMS (APCI⁺) 294 [MH⁺]; [α]_{D}²⁵=−19.4 (c=0.31, MeOH).

Preparation 20

### tert-Butyl(3S,4S)-3,4-dimethoxy-4-phenylpiperidine-1-carboxylate

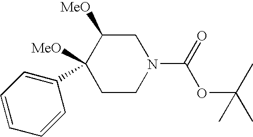

The title compound was formed from the diol of preparation 19 according to the method of preparation 17. ¹H NMR (CDCl₃, 400 MHz) δ 1.49 (s, 9H), 1.98-2.12 (m, 2H), 3.11 (s, 3H), 3.16 (s, 3H), 3.12-3.22 (m, 2H), 3.94 (br, 1H), 4.13 (br, 2H), 7.28-7.32 (m, 1H), 7.35-7.39 (m, 2H), 7.42-7.45 (m, 2H); LRMS (APCI⁺) 322 [MH⁺].

Preparation 21

### (3S,4S)-3,4-Dimethoxy-4-phenylpiperidine hydrochloride

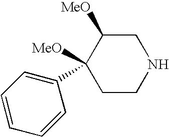

The title compound was formed from the protected piperidine of preparation 20 according to the method of preparation 18. ¹H NMR (CD₃OD, 400 MHz) δ 2.37 (m, 2H), 3.11 (s, 3H), 3.19 (s, 3H), 3.23 (dd, 1H), 3.25 (dd, 1H), 3.29 (m, 2H), 3.66 (dd, 1H), 7.34-7.38 (m, 1H), 7.41-7.50 (m, 4H); LRMS (APCI⁺) 222 [MH⁺].

Preparation 22

### (3S,4S)-3,4-Dihydroxy-4-phenylpiperidine hydrochloride

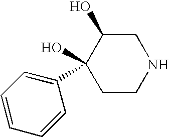

The title compound was formed from the protected piperidine of preparation 19 according to the method of preparation 18. ¹H NMR (CD₃OD, 400 MHz) δ 1.95 (dt, 1H), 2.22 (m, 1H), 3.19-3.38 (m, 4H), 4.21 (dd, 1H), 7.28 (m, 1H), 7.36-7.40 (m, 2H), 7.52-7.56 (m, 2H); LRMS (APCI⁺) 194 [MH⁺].

Preparation 23

### tert-Butyl(3R,4R)-4-hydroxy-3-methoxy-4-phenylpiperidine-1-carboxylate

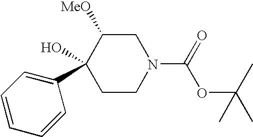

A solution of sodium hydroxide (544 mg, 13.6 mmol) in water (3.4 mL) was added to a solution of tert-butyl(3R,4R)-3,4-dihydroxy-4-phenylpiperidine-1-carboxylate (from preparation 16) (200 mg, 0.68 mmol) in toluene (3.4 mL) followed by methyl iodide (0.85 mL, 13.6 mmol) and tetrabutylammonium hydrogen sulfate (231 mg, 0.68 mmol). The mixture was stirred vigorously at room temperature for 18 hours then diluted with water (20 mL) and extracted with dichloromethane (3×20 mL). The combined organic layers were dried (MgSO₄) and evaporated. The residue was purified by column chromatography (silica) eluting with pentane/ethyl acetate (100% pentane increasing polarity to 30% EtOAc in pentane) to give the title compound as a colourless oil (200 mg, 96%). ¹H NMR (CD₃OD, 400 MHz) δ 1.50 (s, 9H), 1.68 (dt, 1H), 1.93 (td 1H), 3.03 (br, 1H), 3.11 (s, 3H), 3.17 (br, 1H), 3.57 (dd, 1H), 3.85-3.90 (m, 1H), 4.19 (br, 1H), 7.23 (tt, 1H), 7.34 (t, 2H), 7.51 (dd, 2H); LRMS (APCI⁺) 208 [MH⁺-Boc].

Preparation 24

### (3R,4R)-3-methoxy-4-phenylpiperidin-4-ol hydrochloride

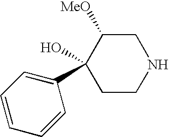

The title compound was formed from the protected piperidine of preparation 23 according to the method of preparation 18. ¹H NMR (CD₃OD, 400 MHz) δ 1.93 (dt, 1H), 2.19-2.27 (m, 1H), 3.12 s, 3H, 3.16-3.33 (m, 2H), 3.47 (dd, 1H), 3.88 (dd, 1H), 4.62 (br s, 1H), 7.30 (tt, 1H), 7.40 (t, 2H), 7.54 (d, 2H); LRMS (APCI⁺) 208 [MH⁺].

Preparation 25

### tert-butyl(3R,4R)-3-ethoxy-4-hydroxy-4-phenylpiperidine-1-carboxylate

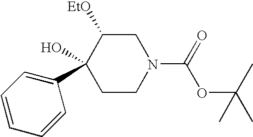

The title compound was formed from the diol of preparation 16 according to the method of preparation 23 using ethyl iodide instead of methyl iodide. ¹H NMR (CD₃OD, 400 MHz) δ 0.89 (t, 3H), 1.50 (s, 9H), 1.68 (dt, 1H), 1.97 (td, 1H), 3.04-3.22 (m, 3H), 3.36-3.43 (m, 1H), 3.60 (dd, 1H), 3.83-3.92 (m, 1H), 4.09-4.16 (br, 1H), 7.23 (tt, 1H), 7.33 (t, 2H), 7.51 (d, 2H); LRMS (APCI⁺) 222 [MH⁺-Boc].

Preparation 26

### (3R,4R)-3-ethoxy-4-phenylpiperidin-4-ol hydrochloride

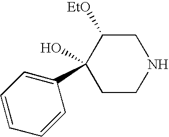

The title compound was formed from the protected piperidine of preparation 25 according to the method of preparation 18. ¹H NMR (CD₃OD, 400 MHz) δ 0.89 (t, 3H), 1.26-1.36 (m, 1H), 1.93 (dt, 1H), 2.23-2.31 (m, 1H), 3.30-3.11 (m, 1H), 3.17-3.45 (m, 4H), 3.92 (dd, 1H), 7.30 (t, 1H), 7.39 (t, 2H), 7.54 (d, 2H); LRMS (APCI⁺) 222 [MH⁺].

Preparation 27

### tert-Butyl(3S,4S)-4-(4-fluorophenyl)-3,4-dihydroxypiperidine-1-carboxylate

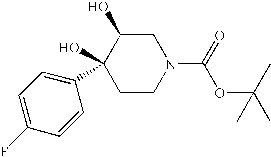

According to the method of preparation 16, but using AD-mix α instead of AD-mix β, tert-butyl 4-(4-fluorophenyl)-3,6-dihydropyridine-1(2H)-carboxylate (prepared according to Synthesis 1991 (11), 993-995) was converted to the title compound. ¹H NMR (CDCl₃, 400 MHz) δ 1.45 (s, 9H), 1.60-1.95 (br, 3H), 2.70 (br, 1H), 2.97 (br, 1H), 3.13 (br, 1H), 3.95 (br, 1H), 4.03 (br, 1H ), 4.17 (br, 1H), 7.05 (m, 2H), 7.43(m, 2H); LRMS (APCI⁺) 312 [MH⁺]; [α]_{D}²⁵=−19.6 (c=0.24, MeOH).

Preparation 28

### tert-Butyl(3S,4S)-4-(4-fluorophenyl)-3,4-dimethoxypiperidine-1-carboxylate

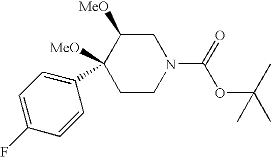

The title compound was formed from the diol of preparation 27 according to the method of preparation 17. ¹H NMR (CDCl₃, 400 MHz) δ 1.46 (s, 9H), 1.93-2.12 (br m, 2H), 2.97-3.22 (br m, 3H), 3.10 (s, 3H), 3.12 (s, 3H),3.95 (br, 1H), 4.20 (br, 1H), 7.03 (m, 2H), 7.42 (m, 2H); LRMS (APCI⁺) 340 [MH⁺].

Preparation 29

### (3S,4S)-4-(4-Fluorophenyl)-3,4-dimethoxypiperidine

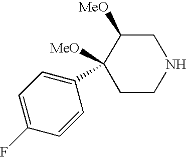

The title compound was formed from the protected piperidine of preparation 28 according to the method of preparation 18. ¹H NMR (CD₃OD, 400 MHz) δ 2.37 (m, 2H), 3.10 (s, 3H), 3.20 (s, 3H), 3.20-3.38 (m, 4H), 3.62 (m, 1H), 7.18 (m, 2H), 7.50 (m, 2H); LRMS (APCI⁺) 240 [MH⁺]; [α]_{D}²⁵=+24.5 (c=0.21, MeOH).

Preparation 30

### tert-Butyl(3R,4R)-4-(4-fluorophenyl)-3,4-dihydroxypiperidine-1-carboxylate

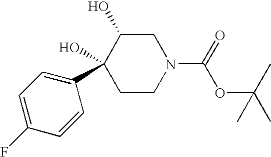

According to the method of preparation 16, tert-butyl 4-(4-fluorophenyl)-3,6-dihydropyridine-1(2H)-carboxylate (prepared according to Synthesis 1991 (11), 993-995) was converted to the title compound. ¹H NMR (CDCl₃, 400 MHz) δ 1.45 (s, 9H), 1.65 (br, 1H), 1.82 (br, 2H), 2.68 (br, 1H), 2.97 (br, 1H), 3.13 (br, 1H), 3.95 (br, 1H), 4.03 (br, 1H), 4.17 (br, 1H), 7.05 (m, 2H), 7.43 (m, 2H); LRMS (APCI⁺) 312 [MH⁺]; [α]_{D}²⁵=+25.7 (c=0.23, MeOH).

Preparation 31

### tert-Butyl(3R,4R)-4-(4-fluorophenyl)-3,4-dimethoxypiperidine-1-carboxylate

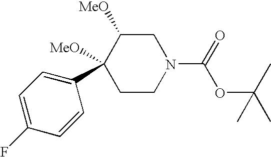

The title compound was formed from the diol of preparation 30 according to the method of preparation 17. ¹H NMR (CDCl₃, 400 MHz) δ 1.46 (s, 9H), 1.93-2.12 (br m, 2H), 2.97-3.22 (br m, 3H), 3.10 (s, 3H), 3.12 (s, 3H),3.95 (br, 1H), 4.20 (br, 1H), 7.03 (m, 2H), 7.42 (m, 2H); LRMS (APCI⁺) 340 [MH⁺].

Preparation 32

### (3R,4R)-4-(4-Fluorophenyl)-3,4-dimethoxypiperidine

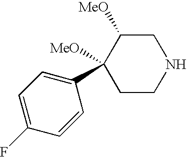

The title compound was formed from the protected piperidine of preparation 31 according to the method of preparation 18. ¹H NMR (CD₃OD, 400 MHz) δ 2.37 (m, 2H), 3.10 (s, 3H), 3.20 (s, 3H), 3.20-3.38 (m, 4H), 3.62 (m, 1H), 7.18 (m, 2H), 7.50 (m, 2H); LRMS (APCI⁺) 240 [MH⁺]; [α]_{D}²⁵=−20.7 (c=0.19, MeOH).

Preparation 33

### tert-Butyl(3S,4S)-4-(4-fluorophenyl)-4-hydroxy-3-methoxypiperidine-1-carboxylate

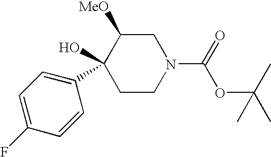

The title compound was formed from the diol of preparation 27 according to the method of preparation 23. ¹H NMR (CD₃OD, 400 MHz) δ 1.45 (s, 9H), 1.65 (m, 1H), 1.90 (m, 1H), 3.00 (br, 1H), 3.10 (s, 3H), 3.08-3.22 (br m, 1H), 3.53 (m, 1H), 3.87 (m, 1H), 4.20 (br, 1H), 7.03 (m, 2H), 7.52 (m, 2H); LRMS (APCI⁺) 326 [MH⁺].

Preparation 34

### (3R,4R)-4-(4-Fluorophenyl)-3,4-dimethoxypiperidine

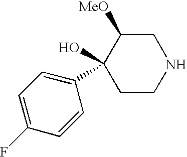

The title compound was formed from the protected piperidine of preparation 33 according to the method of preparation 18. ¹H NMR (CD₃OD, 400 MHz) δ 1.95 (m, 1H), 2.20 (m, 1H), 3.13 (s, 3H), 3.15-3.40 (m, 3H), 3.48 (m, 1H), 3.81 (m, 1H), 7.10 (m, 2H), 7.55 (m, 2H); LRMS (APCI⁺) 226 [MH⁺].

Biological Data

The compound of Example 8 above (first disclosed in Provisional U.S. Patent Application 60/706,191, applicant's reference PC 33020, mentioned above),

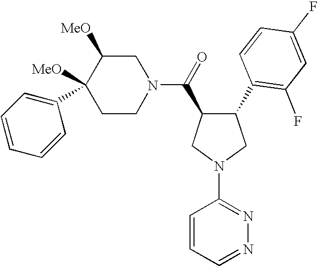

was tested in the dog urethral pressure model (Test A) described above. The compound was dissolved in saline (vehicle) and administered by i.v. infusion over a period of 15 minutes, with at least 5 urethral pressure measurements being taken at each dose level during infusion and for 15 minutes post-infusion. The results are shown in the following table.

| Compound | Mean peak urethral pressure | PUP increase vs. |
|---|---|---|
| dose | (PUP) | baseline |
| (mg/kg) | (mmHg) | (%) |
| 0 (baseline; | 30 | 0 |
| vehicle) | | |
| 1.0 | 32 | 7 |
| 2.0 | 36 | 20 |
| 3.0 | 35 | 17 |

The results indicate that the test compound is able to increase the peak urethral pressure, and so that it is likely to be useful in the treatment of lower urinary tract dysfunction, particularly urinary incontinence.

1. A method of treating lower urinary tract dysfunction in a patient, which method comprises administering to said patient a therapeutically-effective amount of an MC4 receptor agonist.

2. The method of claim 1, wherein the MC4 receptor agonist has the general formula (I) 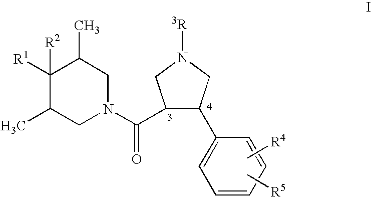
or a pharmaceutically acceptable salt, hydrate, solvate, isomer or prodrug thereof,
wherein R¹ is selected from: —(C₁-C₆)alkyl, —(C₂-C₆)alkenyl, —(C₂-C₆)alkynyl, —(C₃-C₈)cycloalkyl, —(C₅-C₈)cycloalkenyl, —(C₁-C₂)alkyl(C₃-C₈)cycloalkyl, aryl, —(C₁-C₂)alkylaryl, heterocyclic, or —(C₁-C₂)alkylheterocyclic groups
wherein each of the foregoing R¹ groups is optionally substituted by one or more groups selected from: —(C₁-C₄)alkyl, —(CH₂)ₘ(C₃-C₅)cycloalkyl, halogen, —(CH₂)ₘOR⁶, —CN, —C(O)OR⁶, —(CH₂)ₘNR⁷SO₂R⁸, CF₃, CH₂CF₃, OCF₃ or OCH₂CF₃ wherein m=0, 1 or 2;
R² is H, OH or OCH₃;
R³ is selected from: H, —(C₁-C₆)alkyl, —(C₂-C₆)alkenyl, —(C₂-C₆)alkynyl, —(C₃-C₈)cycloalkyl, —(C₅-C₈)cycloalkenyl, —(C₁-C₂)alkyl(C₃-C₈)cycloalkyl, aryl, —(C₁-C₂)alkylaryl, heterocyclic, or —(C₁-C₂)alkylheterocyclic groups
wherein each of the latter ten R³ groups is optionally substituted by one or more groups selected from: —OH, —(C₁-C₄)alkyl, —(CH₂)ₙ(C₃-C₅)cycloalkyl, halogen, —CN, —(CH₂)ₙOR⁶ or —(CH₂)ₙNR⁷R⁸ wherein n=0, 1 or 2;
R⁴ is selected from: —H, —(C₁-C₄)alkyl, —(C₂-C₄)alkenyl, —(C₂-C₄)alkynyl, —(CH₂)ₚ(C₃-C₅)cycloalkyl, —(CH₂)ₚ(C₅)cyclo-alkenyl, halogen, —(CH₂)ₚOR⁶, (CH₂)ₚNR⁷R⁸, —CN, —C(O)R⁶, —C(O)OR⁶, —C(O)NR⁷R⁸, —(CH₂)ₚNR⁷SO₂R⁸, CF₃, CH₂CF₃, OCF₃ or OCH₂CF₃ groups wherein p=0, 1 or 2;
R⁵ is selected from: —(C₁-C₄)alkyl, —(C₂-C₄)alkenyl, —(C₂-C₄)alkynyl, —(CH₂)ₚ(C₃-C₅)cycloalkyl, —(CH₂)ₚ(C₅)cyclo-alkenyl, halogen, —(CH₂)ₚOR⁶, —(CH₂)ₚNR⁷R⁸, —CN, —C(O)R⁶, —C(O)OR⁶, —C(O)NR⁷R⁸, —(CH₂)ₚNR⁷SO₂R⁸, CF₃, CH₂CF₃, OCF₃ or OCH₂CF₃ groups wherein p=0, 1 or 2;
or R⁴ and R⁵ can together form a fused 5- to 7-membered saturated or unsaturated ring;
R⁶, R⁷ and R⁸ are each independently selected from H, CH₃ or CH₂CH₃;
and wherein the heterocyclic groups of R¹ and R³ are independently selected from 4- to 10-membered ring systems containing up to 4 heteroatoms independently selected from O, N or S.

3. The method of claim 2, wherein R¹ is selected from: —(C₁-C₆)alkyl, —(C₃-C₈)cycloalkyl, —(C₁-C₂)alkyl(C₃-C₈)cycloalkyl, phenyl, —(C₁-C₂)alkylaryl, heterocyclic, or —(C₁-C₂)alkylheterocyclic groups
wherein each of the foregoing R¹ groups is optionally substituted by one or more groups selected from: —(C₁-C₄)alkyl, halogen, —(CH₂)ₘOR⁶, CN, CF₃ or OCF₃, wherein m=1 or 2;
R² is OH;
R³ is selected from: —H, —(C₁-C₆)alkyl, —(C₃-C₈)cycloalkyl, —(C₁-C₂)alkyl(C₃-C₈)cycloalkyl, aryl, —(C₁-C₂)alkylaryl, heterocyclic, or —(C₁-C₂)alkyl heterocyclic groups
wherein each of the latter seven R³ groups is optionally substituted by one or more groups selected from: —OH, —(C₁-C₄)alkyl, —(CH₂)ₙ(C₃-C₅)cycloalkyl, halogen, CN, —(CH₂)ₙOR⁶ or —(CH₂)ₙNR⁷R⁸ wherein n=0, 1 or 2;
R⁴ is selected from: —H, —(C₁-C₄)alkyl, —(CH₂)ₚ(C₃-C₅)cycloalkyl, halogen, —(CH₂)ₚOR⁶, —(CH₂)ₚNR⁷R⁸, —CN, —C(O)R⁶, —C(O)OR⁶, —C(O)NR⁷R⁸, —(CH₂)ₚNR⁷SO₂R⁸, CF₃, CH₂CF₃, OCF₃ or OCH₂CF₃ groups wherein p=0, 1 or 2;
R⁵ is selected from: —(C₁-C₄)alkyl, —(CH₂)ₚ(C₃-C₅)cycloalkyl, halogen, —(CH₂)ₚOR⁶, (CH₂)ₚNR⁷R⁸, CN, C(O)R⁶, C(O)OR⁶, CONR⁷R⁸, (CH₂)ₚNR⁷SO₂R⁸, CF₃, CH₂CF₃, OCF₃ or OCH₂CF₃ groups wherein p=0, 1 or 2;
R⁶, R⁷ and R⁸ are each independently selected from H, CH₃ or CH₂CH₃;
wherein the heterocyclic group of R³ is selected from mono-cyclic 5- to 6-membered ring systems containing up to 2 heteroatoms independently selected from O or N and combinations thereof.
and wherein the heterocyclic group of R¹ is selected from mono-cyclic 5- to 6-membered ring systems containing up to 1 heteroatoms independently selected from O or N.

4. The method of claim 2, wherein the compound is of general formula (IC) 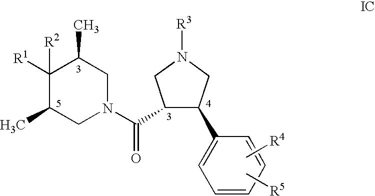
wherein:
R¹ is a phenyl, 3-fluorophenyl, 4-fluorophenyl, 2,6-difluorophenyl, 2,4-difluorophenyl, 3,4-difluorophenyl or pyridin-2-yl group;
R² is OH;
R³ is t-butyl;
R⁴ is selected from: H or F and R⁵ is selected from: F or Cl.

5. The method according to claim 2, wherein the compound of formula (I) is (3R,4R,5S)-1-{[(3S,4R)-1-tert-butyl-4-(2,4-difluorophenyl)pyrrolidin-3-yl]carbonyl}-3,5-dimethyl-4-phenylpiperidin-4-ol, having the formula 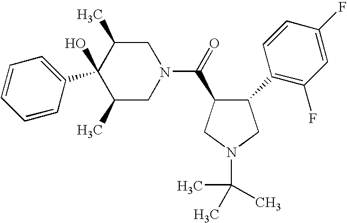
or a pharmaceutically acceptable salt, hydrate, solvate, isomer or prodrug thereof.

6. The method of claim 1, wherein the MC4 receptor agonist compound has the general formula (Ia) 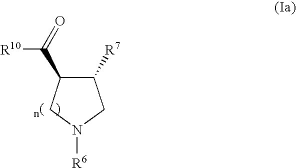
wherein:
n is 1 or 2;
R⁶ is selected from H, C₁-C₆alkyl, C₃-C₈cycloalkyl, aryl, heterocyclyl, heteroaryl, C(O)C₁-C₆alkyl and CO₂C₁-C₆alkyl, wherein said moieties may be optionally substituted with one or more substituents independently selected from halo, CN, C₁-C₄alkyl and C₁-C₄alkoxy;
R⁷ is selected from pyridinyl and phenyl, wherein said pyridinyl or said phenyl is substituted by 1-3 groups independently selected from halo, CN, CF₃, OCF₃, OC₁-C₄alkyl and C₁-C₄alkyl;
R¹⁰ is a substituted piperidine group of formula (IIa): 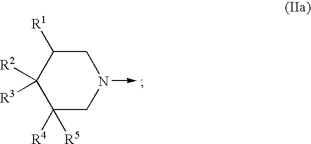
wherein
R¹ and R⁴ are each independently selected from H, C₁-C₄alkyl, OH, O(C₁-C₄alkyl), CH₂OCH₃ and NR⁸R⁹;
R² is selected from H, OH, OC₁-C₄alkyl and NR⁸R⁹;
R³ is selected from aryl or heteroaryl, wherein said moieties are optionally substituted with one or more substituents independently selected from halo, CN, CF₃, OCF₃, O(C₁-C₄alkyl), and C₁-C₄alkyl;
R⁵ is selected from H and C₁-C₄alkyl;
R⁸ is selected from H and C₁-C₄alkyl, wherein said C₁-C₄alkyl is optionally substituted with OH or OCH₃;
R⁹ is selected from H, C₁-C₄alkyl, SO₂C₁-C₄alkyl, C(O)C₁-C₄alkyl;
wherein aryl means a six or ten membered aromatic hydrocarbon ring which is optionally fused to another six or ten membered aromatic hydrocarbon ring;
wherein heteroaryl means a 5 or 6 membered aromatic ring, containing from 1 to 4 heteroatoms, said heteroatoms each independently selected from O, S and N, wherein said aromatic ring may be optionally fused to an aryl or second, non-fused, aromatic heterocyclic ring;
wherein heterocyclyl means a 4 to 7 membered saturated or partially saturated ring, containing from 1 to 2 heteroatoms each independently selected from O, S and N;
wherein halo means Cl, F, Br or I;
and pharmaceutically acceptable salts, hydrate, solvates, polymorphs and prodrugs thereof, with the provisos that:
R¹, R⁴ and R⁵ are not all simultaneously H;
when R¹ is methyl and R⁴ is H, then R⁵ is not methyl;
when R⁴ is methyl and R⁵ is H, then R¹ is not methyl; and
when R⁵ is methyl and R⁴ is H, then R¹ is not methyl.

7. The method of claim 6, wherein:
n is 1;
R¹ is selected from H, methyl, OH, OCH₃ and OC₂H₅;
R² is selected from OH, OCH₃ and OC₂H₅;
R³ is selected from phenyl or pyridinyl, wherein said moieties are optionally substituted with one or more substituents independently selected from F, Cl, CN and CF₃;
R⁴ is selected from H, methyl, OH, OCH₃ and OC₂H₅;
R⁵ is selected from H and methyl;
R⁶ is selected from C₁-C₄alkyl, tetrahydropyranyl, tetrahydrofuranyl, pyrimidinyl pyridinyl and pyridazinyl, wherein each of said moieties is optionally substituted with one or more substituents independently selected from halo, CN, methyl and OCH₃;
R⁷ is selected from pyridinyl and phenyl, wherein said pyridinyl or said phenyl is substituted by 1-2 groups independently selected from Cl, F, CN and OCH₃;
R⁸ is selected from H, methyl and ethyl; and
R⁹ is selected from H and methyl.

8. The method of claim 6, wherein R⁶ is selected from the following group:

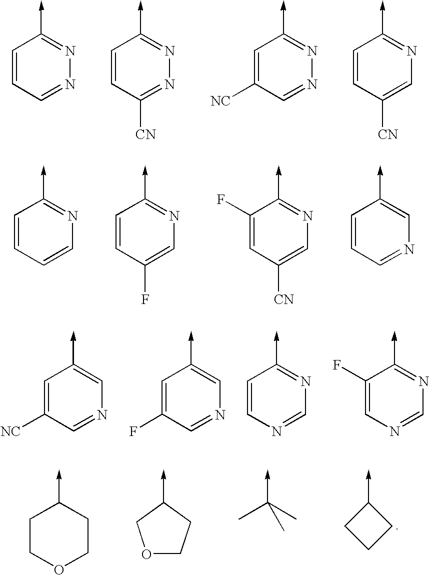

9. The method of claim 6, wherein R⁷ is selected from the following group:

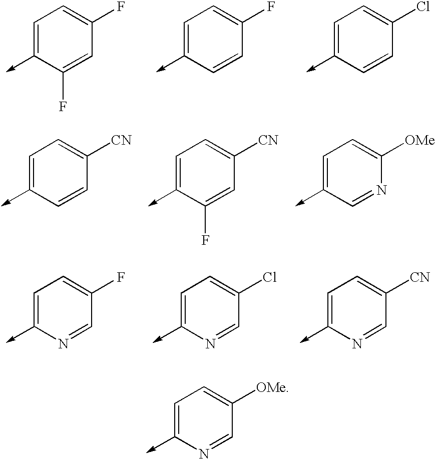

10. The method of claim 6, wherein R¹⁰ is selected from the following group:

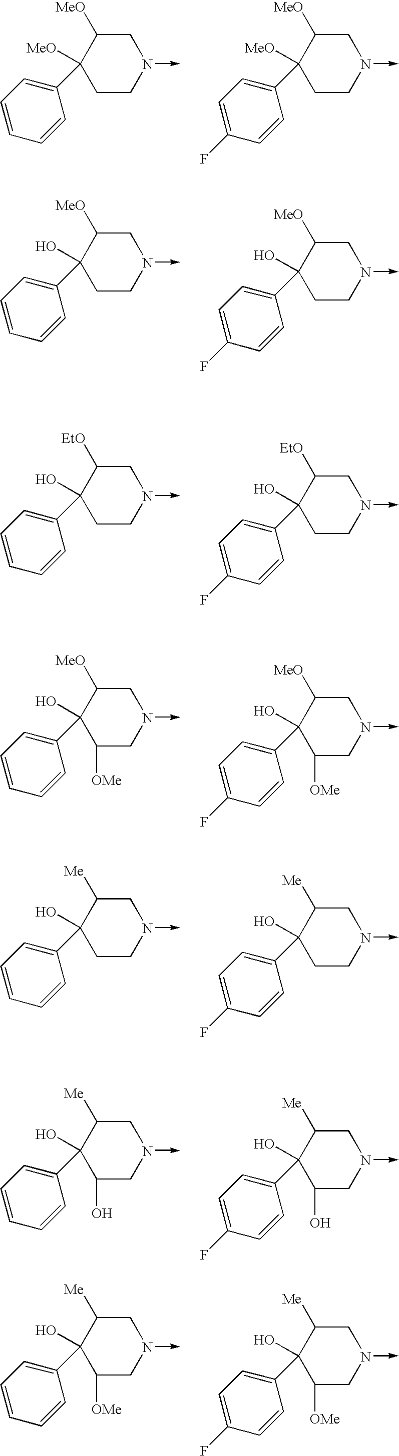

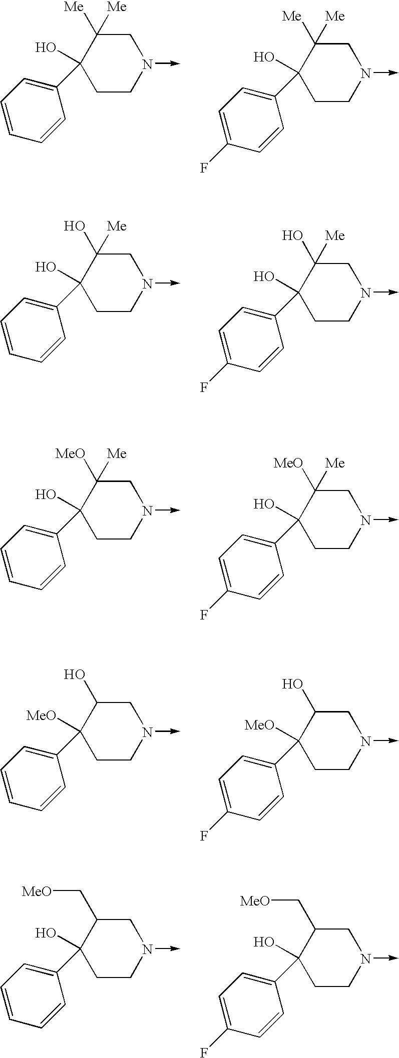

11. The method of claim 6, wherein the compound of formula Ia is 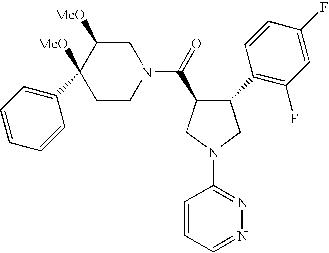
or a pharmaceutically acceptable salt, hydrate, solvate, polymorph or prodrug thereof.

12. The method of claim 1, wherein the MC4 receptor agonist compound is a compound of formula (Ib), 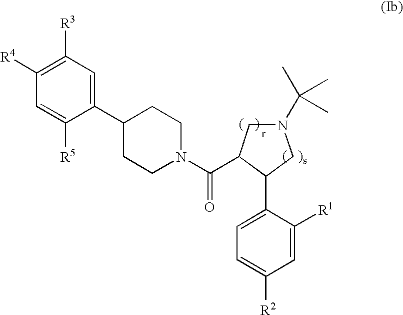
or a pharmaceutically acceptable salt thereof; wherein
R¹ and R² are selected from the group consisting of:
(1) halogen,
(2) CF₃,
(3) CH₃, and
(4) OCH₃;
R³ and R⁴ are independently selected from the group consisting of:
(1) C₁₋₄ alkyl,
(2) —CF₃,
(3) halogen,
(4) —OC₁₋₄ alkyl,
(5) —OCF₃,
(6) —OCHF₂,
(7) —S(O)ₚC₁₋₄ alkyl, and
(8) —CN,
wherein alkyl is unsubstituted or substituted with one to three substituents independently selected from halogen, hydroxy, oxo, C₁₋₄ alkyl, trifluoromethyl, and C₁₋₄ alkoxy, or wherein the R³ and R⁴ substitutents taken together with the carbons to which they are attached form a 4-6 membered ring optionally containing a heteroatom selected from O, S, —NH, and —NC₁₋₄alkyl;
R⁵ is selected from the group consisting of:
(1) —C₁₋₈ alkyl,
(2) —(CH₂)ₙ-heteroaryl,
(3) —(CH₂)ₙheterocycloalkyl,
(4) halogen,
(5) —OR⁶,
(6) —(CH₂)ₙC(O)R⁶,
(7) —(CH₂)ₙOC(O)R⁶,
(8) —(CH₂)ₙC(O)OR⁶,
(9) —(CH₂)ₙC≡N,
(10) —(CH₂)ₙN(R⁶)₂,
(11) —(CH₂)ₙC(O)N(R⁶)₂,
(12) —(CH₂)ₙNR⁶C(O)R⁶,
(13) —(CH₂)ₙNR⁶C(O)OR⁶,
(14) —(CH₂)ₙNR⁶C(O)-heteroaryl,
(15) —(CH₂)ₙNR⁶C(O)N(R⁶)₂,
(16) —(CH₂)ₙNR⁶-heteroaryl,
(17) —(CH₂)ₙC(O)NR⁶N(R⁶)₂,
(18) —(CH₂)ₙC(O)NR⁶NR⁶C(O)R⁶,
(19) —(CH₂)ₙNR⁶S(O)ₚR⁶,
(20) —(CH₂)ₙS(O)ₚN(R⁶)₂,
(21) —(CH₂)ₙS(O)ₚR⁶,
(22) —O(CH₂)ₙC(O)N(R⁶)₂,
(23) —(CH₂)ₙCF₃, and
(24) —O(CH₂)ₙCF₃,
wherein heteroaryl is unsubstituted or substituted with one to three substituents independently selected from halogen, hydroxy, C₁₋₄ alkyl, trifluoromethyl, and C₁₋₄ alkoxy, and wherein any alkyl, heterocycloalkyl, and methylene (CH₂) carbon atom in R⁵ is unsubstituted or substituted with one to two substituents independently selected from halogen, hydroxy, oxo, C₁₋₄ alkyl, trifluoromethyl, and C₁₋₄ alkoxy, or two substituents on the same R⁵ carbon atom are taken together with the carbon atom to form a 3- to 6-membered ring;
each R⁶ is independently selected from the group consisting of:
(1) hydrogen,
(2) C₁₋₈ alkyl,
(3) phenyl,
(4) heteroaryl,
(5) —(CH₂)ₙheterocycloalkyl, and
(6) C₃₋₆ cycloalkyl,
wherein alkyl, phenyl, heteroaryl, heterocycloalkyl, and cycloalkyl are unsubstituted or substituted with one to three substituents independently selected from halogen, C₁₋₄ alkyl, hydroxy, and C₁₋₄ alkoxy, or two R⁶ substituents together with the atoms to which they are attached form a 4- to 8-membered mono- or bicyclic ring system optionally containing an additional heteroatom selected from O, S, —NH, and —NC₁₋₄ alkyl;
r is 1 or 2;
s is 0, 1, or 2;
n is 0, 1, 2, 3, or 4; and
p is 0, 1, or 2.

13. The method of claim 1, wherein the MC4 receptor agonist compound is a compound of formula (Id), 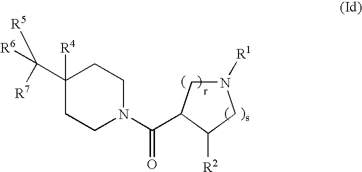
or a pharmaceutically acceptable salt thereof, wherein
R¹ is selected from the group consisting of:
(1) hydrogen,
(2) amidino,
(3) —C₁₋₄ alkyliminoyl,
(4) —C₁₋₈ alkyl,
(5) —(CH₂)ₙ—C₃₋₇ cycloalkyl,
(6) —(CH₂)ₙheterocycloalkyl,
(7) —(CH₂)ₙ-phenyl,
(8) —(CH₂)ₙ-naphthyl, and
(9) (CH₂)ₙ-heteroaryl,
wherein phenyl, naphthyl, and heteroaryl are unsubstituted or substituted with one to three substituents independently selected from R³, and alkyl, cycloalkyl, and heterocycloalkyl are unsubstituted or substituted with one to three substitutents independently selected from R³ and oxo;
R² is selected from the group consisting of:
(1) phenyl,
(2) naphthyl, and
(3) heteroaryl,
wherein phenyl, naphthyl, and heteroaryl are unsubstituted or substituted with one to three substitutuents independently selected from R⁹;
each R³ is independently selected from the group consisting of:
(1) —C₁₋₈ alkyl,
(2) —(CH₂)ₙ-phenyl,
(3) —(CH₂)ₙ-heteroaryl,
(4) —(CH₂)ₙheterocycloalkyl,
(5) —(CH₂)ₙC₃₋₇ cycloalkyl,
(6) halogen,
(7) —OR⁸,
(8) —(CH₂)ₙC≡N,
(9) —(CH₂)ₙN(R⁸)₂,
(10) —(CH₂)ₙC(O)N(R⁸)₂,
(11) —(CH₂)ₙC(O)NR⁸N(R⁸)₂,
(12) —(CH₂)ₙC(O)NR⁸NR⁸C(O)R⁸, and
(13) —(CH₂)ₙCF₃,
wherein phenyl and heteroaryl are unsubstituted or substituted with one to three substituents independently selected from halogen, hydroxy, C₁₋₄ alkyl, trifluoromethyl, and C₁₋₄ alkoxy, and wherein any alkyl, cycloalkyl, heterocycloalkyl, and methylene (CH₂) carbon atom in R³ is unsubstituted or substituted with one to two substituents independently selected from halogen, hydroxy, oxo, C₁₋₄ alkyl, trifluoromethyl, and C₁₋₄ alkoxy, or two R³ substituents on the same carbon atom are taken together with the carbon atom to form a cyclopropyl group;
R⁴ is selected from the group consisting of:
(1) hydrogen, and
(2) —C₁₋₆ alkyl,
(3) —OC₁₋₆ alkyl, and
(4) —(CH₂)ₙN(R⁸)C(O)R⁸;
R⁵ is selected from the group consisting of:
(1) —CF₃,
(2) —C₁₋₆ alkyl,
(3) —C₂₋₈ alkenyl,
(4) —C₂₋₈ alkynyl,
(5) —OC₁₋₈ alkyl,
(6) —(CH₂)ₙC₃₋₇cycloalkyl,
(7) —(CH₂)ₙheterocycloalkyl,
(8) —(CH₂)ₙ-phenyl,
(9) —(CH₂)ₙ-naphthyl,
(10) —(CH₂)ₙheteroaryl, and
(11) —(CH₂)ₙC₃₋₇bicycloalkyl,
wherein phenyl, naphthyl, and heteroaryl are unsubstituted or substituted with one to three substituents independently selected from R³, and alkyl, alkenyl, alkynyl, cycloalkyl, heterocycloalkyl, and bicycloalkyl are unsubstituted or substituted with one to three substituents independently selected from R³ and oxo, and wherein any methylene (CH₂) in R⁵ is unsubstituted or substituted with one to two substituents independently selected from halogen, hydroxy, oxo, and C₁₋₄ alkyl;
R⁶ is selected from the group consisting of:
(1) hydrogen,
(2) —C₁₋₆ alkyl, and
(3) —OC₁₋₆ alkyl;
R⁷ is selected from the group consisting of:
(1) —(CH₂)ₙN(R⁸)₂,
(2) —(CH₂)ₙNR⁸C(O)R⁸,
(3) —(CH₂)ₙOR⁸,
(4) —(CH₂)ₙC≡N,
(5) —(CH₂)ₙC(O)OR⁸,
(6) —(CH₂)ₙC(O)N(R⁸)₂,
(7) —(CH₂)ₙNR⁸C(O)N(R⁸)₂,
(8) —(CH₂)ₙNR⁸C(O)heteroaryl,
(9) —(CH₂)ₙheteroaryl,
(10) —(CH₂)ₙNR⁸S(O)ₚR⁸,
(11) —(CH₂)ₙSR⁸, and
(12) —(CH₂)ₙS(O)ₚR⁸,
wherein heteroaryl is unsubstituted or substituted with one to three substituents selected from C₁₋₄ alkyl; and any methylene (CH₂) in R⁷ is unsubstituted or substituted with one to two substituents independently selected from halogen, hydroxyl, oxo, and C₁₋₄ alkyl, or two C₁₋₄ alkyl substituents on any methylene (CH₂) in R⁷ together with the atom to which they are attached form a 3, 4, 5, or 6-membered ring optionally containing an additional heteroatom selected from O, S, —NH, and —NC₁₋₄ alkyl;
each R⁸ is independently selected from the group consisting of:
(1) hydrogen,
(2) —C₁₋₈ alkyl,
(3) —C₂₋₈ alkenyl,
(4) —(CH₂)ₙC₃₋₇ cycloalkyl,
(5) —(CH₂)ₙheterocycloalkyl.
6) —(CH₂)ₙ-phenyl, and
(7) —(CH₂)ₙ-heteroaryl;
each R⁹ is independently selected from the group consisting of:
(1) —C₁₋₈ alkyl,
(2) —C₂₋₈ alkenyl,
(3) —(CH₂)ₙ-phenyl,
(4) —(CH₂)ₙ-naphthyl,
(5) —(CH₂)ₙ-heteroaryl,
(6) —(CH₂)ₙheterocycloalkyl,
(7) —(CH₂)ₙC₃₋₇ cycloalkyl,
(8) halogen,
(9) —OR⁸,
(10) —(CH₂)ₙC(O)R⁸,
(11) —(CH₂)ₙOC(O)R⁸,
(12) —(CH₂)ₙC(O)OR⁸,
(13) —(CH₂)ₙC≡N,
(14) NO₂,
(15) —(CH₂)ₙN(R⁸)₂,
(16) —(CH₂)ₙC(O)N(R⁸)₂,
(17) —(CH₂)ₙNR⁸C(O)R⁸,
(18) —(CH₂)ₙNR⁸C(O)OR⁸,
(19) —(CH₂)ₙNR⁸C(O)-heteroaryl,
(20) —(CH₂)ₙNR⁸C(O)N(R⁸)₂,
(21) —(CH₂)ₙC(O)NR⁸N(R⁸)₂,
(22) —(CH₂)ₙC(O)NR⁸NR⁸C(O)R⁸,
(23) —(CH₂)ₙNR⁸S(O)ₚR⁸,
(24) —(CH₂)ₙS(O)ₚN(R⁸)₂,
(25) —(CH₂)ₙS(O)ₚR⁸,
(26) —O(CH₂)ₙC(O)N(R⁸)₂,
(27) —(CH₂)ₙCF₃, and
(28) —O(CH₂)ₙCF₃,
wherein alkenyl, phenyl, naphthyl, and heteroaryl are unsubstituted or substituted with one to three substituents independently selected from halogen, hydroxy, C₁₋₄ alkyl, trifluoromethyl, and C₁₋₄ alkoxy, and wherein alkyl, cycloalkyl, heterocycloalkyl, and any methylene (CH₂) carbon atom in R⁹ are unsubstituted or substituted with one or two substituents independently selected from halogen, hydroxy, oxo, C₁₋₄ alkyl, trifluoromethyl, and C₁₋₄ alkoxy, or two R⁹ substituents on the same carbon atom are taken together with the carbon atom to form a cyclopropyl group;
r is 1 or 2;
s is 0, 1 or 2;
n is 0, 1, 2, 3, or 4; and
p is 0, 1, or 2.

14. The method of claim 1, wherein the lower urinary tract dysfunction is selected from:
(i) urinary incontinence, including stress urinary incontinence, urge urinary incontinence and mixed urinary incontinence;
(ii) overactive bladder (OAB), which includes one or more of the symptoms of increased daytime frequency and urgency, and nocturia, which symptoms may or may not result in loss of urine (OAB wet and OAB dry), and urge incontinence; and
(iii) lower urinary tract symptoms (LUTS) comprising one or more of the above symptoms, and, when associated with BPH, at least one of the additional symptoms of terminal dribble, hesitancy, intermittency, straining and poor flow.

15. The method of claim 14, wherein the lower urinary tract dysfunction is urinary incontinence.

16. The method of claim 15, wherein the urinary incontinence is stress urinary incontinence.

17. The method of claim 1, wherein the MC4 receptor agonist compound exhibits a binding constant at the MC4 receptor expressed as a Ki value against AGRP of lower than 100 nM.

18. The method of claim 1, wherein the MC4 receptor agonist compound is able to penetrate into the human central nervous system.

19. The method of claim 1, wherein the MC4 receptor agonist compound has a molecular weight less than 450.

20. The method of claim 1, wherein the MC4 receptor agonist compound has a polar surface area of less than 90 Å².

21. The method of claim 1, wherein the MC4 receptor agonist compound has a log D between 1 and 3.

22. The method of claim 1, wherein the MC4 receptor agonist compound has a pKa between 7.5 and 10.5.
